(19) United States
(12) Patent Application Publication
(10) Pub. No.: US 2007/0021360 A1
(43) Pub. Date: Jan. 25, 2007

(54) Compositions, formulations and kit with anti-sense oligonucleotide and anti-inflammatory steroid and/or obiquinone for treatment of respiratory and lung disesase

(76) Inventors: Jonathan W. Nyce, Titusville, NJ (US); Jonathan Pabalan, Burlington, NJ (US); Douglas Aguilar, Hackensack, NJ (US); Shoreh Miller, Plainsboro, NJ (US); Yukui Li, Cambridge, MA (US); Anthony Sandrasagra, Princeton, NJ (US); Evan Katz, North Brunswick, NJ (US); Lei Tang, Princeton, NJ (US); Syed Shahabuddin, Newton, PA (US)

Correspondence Address: WILSON SONSINI GOODRICH & ROSATI, 650 PAGE MILL ROAD, PALO ALTO, CA, 943041050, US

(21) Appl. No.: 10/475,684

(22) PCT Filed: Apr. 23, 2002
(86) PCT No.: PCT/US02/13135

§ 371 Date: Aug. 31, 2004

Related U.S. Application Data
(60) Provisional application No. 60/286,137, filed on Apr. 24, 2001.

(51) Int. Cl. A61K 48/00; A61K 31/56

(52) U.S. Cl. 514/44; 514/26; 514/171

(57) ABSTRACT

A pharmaceutical composition and formulations comprise preventative, prophylactic or therapeutic amounts of an oligo(s) anti-sense to a specific gene(s) or its corresponding mRNA(s), and a glucocorticoid and/or non-glucocorticoid steroid or a ubiquinone or their salts. The agents, composition and formulations are used for treatment of ailments associated with impaired respiration, bronchoconstriction, lung allergy(ies) or inflammation, and abnormal levels of adenosine, adenosine receptors, sensitivity to adenosine, lung surfactant and ubiquinone, such as pulmonary fibrosis, vasoconstriction, inflammation, allergies, allergic rhinitis, asthma, impeded respiration, lung pain, cystic fibrosis, bronchoconstriction, COPD, RDS, ARDS, cancer, and others. The present treatment is effectively administered by itself for conditions without known therapies, as a substitute for therapies exhibiting undesirable side effects, or in combination with other treatments, e.g. before, during and after other respiratory system therapies, radiation, chemotherapy, antibody therapy and surgery, among others. Each of the agents of this invention may be administered directly into the respiratory system so that they gain direct access to the lungs, or by other effective routes of administration. A kit comprises a delivery device, the agents and instructions for its use.

### BACKGROUND OF THE INVENTION

1. Field of the Invention

This invention concerns itself with compositions, formulations and kits employed for the administration of active agents that are effective for treating respiratory and pulmonary diseases including bronchoconstriction, impaired airways, decreased lung surfactant, asthma, rhinitis, acute respiratory distress syndrome (ARDS), infantile or maternal RDS, chronic obstructive pulmonary disease (COPD), allergies, impeded respiration, lung pain, cystic fibrosis (CF), infectious diseases, cancers such as leukemias, lung and colon cancer, and the like, and diseases whose secondary effects afflict the lungs. The active agents, anti-sense oligonucleotides and steroid agents and/or ubiquinones may be administered preventatively, prophylactically or therapeutically as a single therapy or in conjunction with other therapies.

2. Background of the Invention

Respiratory ailments, associated with a variety of diseases and conditions, are extremely common in the general population, and more so in certain ethnic groups, such as African Americans. In some cases they are accompanied by inflammation, which aggravates the condition of the lungs. Asthma, for example, is one of the most common diseases in industrialized countries. In the United States it accounts for about 1% of all health care costs. An alarming increase in both the prevalence and mortality of asthma over the past decade has been reported, and asthma is predicted to be the preeminent occupational lung disease in the next decade. While the increasing mortality of asthma in industrialized countries could be attributable to the depletion reliance upon beta agonists in the treatment of this disease, the underlying causes of asthma remain poorly understood. Respiratory and pulmonary diseases such as asthma, allergic rhinitis, Acute Respiratory Distress Syndrome (ARDS), including that occurring in pregnant mothers and in premature born infants, pulmonary fibrosis, cystic fibrosis (CF), chronic obstructive pulmonary disease (COPD), and cancer, among others, are common diseases in industrialized countries. In the United States alone they account for extremely high health care costs, and their incidence has recently been increasing at an alarming rate, both in terms of prevalence, morbidity and mortality. In spite of this, their underlying causes still remain poorly understood.

Asthma is a condition characterized by variable, in many instances reversible obstruction of the airways. This process is associated with lung inflammation and in some cases lung allergies. Many patients have acute episodes referred to as “asthma attacks,” while others are afflicted with a chronic condition. The asthmatic process is triggered in some cases by inhalation of antigens by hypersensitive subjects. This condition is generally referred to as “extrinsic asthma.” Other asthmatics have an intrinsic predisposition to the condition, which is thus referred to as “intrinsic asthma,” and may be comprised of conditions of different origin, including those mediated by the adenosine receptor(s), allergic conditions mediated by an immune IgE-mediated response, and others. All asthmas have a group of symptoms, which are characteristic of this condition: bronchoconstriction, lung inflammation and decreased lung surfactant. Existing bronchodilators and anti-inflammatories are currently commercially available and are prescribed for the treatment of asthma. The most common anti-inflammatories, corticosteroids, have considerable side effects but are commonly prescribed nevertheless. Most of the drugs available for the treatment of asthma are, more importantly, barely effective in a small number of patients.

Acute Respiratory Distress Syndrome (ARDS), or stiff lung, shock lung, pump lung and congestive atelectasis, is believed to be caused by fluid accumulation within the lung which, in turn, causes the lung to stiffen. The condition is triggered within 48 hours by a variety of processes that injure the lungs such as trauma, head injury, shock, sepsis, multiple blood transfusions, medications, pulmonary embolism, severe pneumonia, smoke inhalation, radiation, high altitude, near drowning, and others. In general, ARDS occurs as a medical emergency and may be caused by other conditions that directly or indirectly cause the blood vessels to “leak” fluid into the lungs. In ARDS, the ability of the lungs to expand is severely decreased and produces extensive damage to the air sacs and lining or endothelium of the lung. ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. ARDS is commonly diagnosed by testing for symptomatic signs, for example by a simple chest auscultation or examination with a stethoscope that may reveal abnormal symptomatic breath sounds. A preliminary diagnosis of ARDS may be confirmed with chest X-rays and the measurement of arterial blood gas. In some cases ARDS appears to be associated with other diseases, such as acute myelogenous leukemia, with acute tumor lysis syndrome (ATLS) developed after treatment with, e.g. cytosine arabinoside. In general, however, ARDS appears to be associated with traumatic injury, severe blood infections such as sepsis, or other systemic illness, high dose radiation therapy and chemotherapy, and inflammatory responses which lead to multiple organ failure, and in many cases death. In premature babies (“premies”), the lungs are not quite developed and, therefore, the fetus is in an anoxic state during development. Moreover, lung surfactant, a material critical for normal respiration, is generally not yet present in sufficient amounts at this early stage of life; however, premies often hyper-express the adenosine A₁ receptor and/or underexpress the adenosine A₂ₐ receptor and are, therefore, susceptible to respiratory problems including bronchoconstriction, lung inflammation and ARDS, among others. When Respiratory Distress Syndrome (RDS) occurs in premies, it is an extremely serious problem. Preterm infants exhibiting RDS are currently treated by ventilation and administration of oxygen and surfactant preparations. When premies survive RDS, they frequently develop bronchopulmonary dysplasia (BPD), also called chronic lung disease of early infancy, which is often fatal.

The systemic administration of adenosine was found useful for treating SVT, and as a pharmacologic means to evaluate cardiovascular health via an adenosine stress test commonly administered by hospitals and by doctors in private practice. Adenosine administered by inhalation, however, is known to cause bronchoconstriction in asthmatics, possibly due to mast cell degranulation and histamine release, effects which have not been observed in normal subjects. Adenosine infusion has caused respiratory compromise, for example, in patients with COPD. As a consequence of the untoward side effects observed in many patients, caution is recommended in the prescription of adenosine to patients with a variety of conditions, including obstructive lung disease, emphysema, bronchitis, etc, and complete avoidance of its administration to patients with or prone to bronchoconstriction or bronchospasm, such as asthma. In addition, the administration of adenosine must be discontinued in any patient who develops severe respiratory difficulties. It would be of great help if a formulation were to be made available for joint use when adenosine administration is required.

Allergic rhinitis afflicts one in five Americans, accounting for an estimated $4 to 10 billion in health care costs each year, and occurs at all ages. Because many people mislabel their symptoms as persistent colds or sinus problems, allergic rhinitis is probably underdiagnosed. Typically, IgE combines with allergens in the nose to produce chemical mediators, induction of cellular processes, and neurogenic stimulation, causing an underlying inflammation. Symptoms include nasal congestion, discharge, sneezing, and itching, as well as itchy, watery, swollen eyes. Over time, allergic rhinitis sufferers often develop sinusitis, otitis media with effusion, and nasal polyposis that may exacerbate asthma, and is associated with mood and cognitive disturbances, fatigue and irritability. Degranulation of mast cells results in the release of preformed mediators that interact with various cells, blood vessels, and mucous glands to produce the typical rhinitis symptoms. Most early- and late-phase reactions occur in the nose after allergen exposure. The late-phase reaction is seen in chronic allergic rhinitis, with hypersecretion and congestion as the most prominent symptoms. Repeated exposure may cause hypersensitivity to one or many allergens. Sufferers may also become hyperreactive to non-specific triggers, such as cold air or strong odors. Non-allergic rhinitis may be induced by infections, such as viral infections, or associated with nasal polyps, as occurs in patients with aspirin idiosyncrasy. In addition, pregnancy, hypothyroidism, and exposure to occupational factors or medications may cause rhinitis, as well. NARES syndrome, a non-allergic type of rhinitis associated with eosinophils in nasal secretions, typically occurs in middle-aged individuals and is accompanied by loss of smell. Saline is often recommended to improve nasal stuffiness, sneezing, and congestion, since saline sprays usually relieve mucosal irritation or dryness associated with various nasal conditions, minimize mucosal atrophy, and dislodge encrusted or thickened mucus, while causing no side effects, and may be used freely in pregnant patients. In addition, if used immediately before intra-nasal corticosteroid dosing, saline helps prevent local irritation. Anti-histamines often serve as a primary therapy. Terfenadine and astemizole, two non-sedating anti-histamines, however, have been associated with a ventricular arrhythmia known as Torsades de Points, usually in interaction with other medications such as ketoconazole and erythromycin, or secondary to an underlying cardiac problem. Up to date, loratadine, another nonsedating anti-histamine, and cetirizine have not been associated with serious adverse cardiovascular events. Cetirizine's most common side effect, however, is drowsiness. Clartin, for example, may be effective in relieving sneezing, runny nose, and nasal, ocular and palatal itching in a low percentage of patients, although not approved for this indication or asthma. Anti-histamines are typically combined with a decongestant to help relieve nasal congestion. Sympathomimetic medications are used as vasoconstrictors and decongestants, the most common being pseudoephedrine, phenylpropanolamine and phenylephrine. These agents, however, often cause hypertension, palpitations, tachycardia, restlessness, insomnia and headache. Topical decongestants are recommended for limited periods because their overuse results in nasal dilatation. Anti-cholinergic agents, such as cromolyn, have a role in patients with significant rhinorrhea or in specific cases, such as “gustatory rhinitis”, which is usually associated with ingestion of spicy foods, and have been used on the common cold Sometimes the Cromolyn spray produces sneezing, transient headache, and even nasal burning. Topical and nasal spray corticosteroids such as Vancenase are effective agents in the treatment of rhinitis, especially for symptoms of congestion, sneezing and runny nose, but sometimes may cause irritation stinging, burning, sneezing, and local bleeding. Topical steroids are generally more effective than Cromolyn sodium, particularly in the treatment of NARES, but side effects sometimes limit their usefulness. Immunotherapy, while expensive and inconvenient, often provides substantial benefits, especially the use of drugs such as blocking antibodies, and those that alter cellular histamine release, and result in decreased IgE. Presently available treatments, such as propranolol, verapamil, and adenosine, may help to minimize symptoms. Verapamil is most commonly used but it has several shortcomings, since it causes or exacerbates systemic hypotension, congestive heart failure, bradyarrhythmias, and ventricular fibrillation. Verapamil, however, crosses the placenta and has been shown to cause fetal bradycardia, heart block, depression of contractility, and hypotension. Adenosine has several advantages over verapamil, including rapid onset, brevity of side effects, theoretical safety, and probable lack of placental transfer, but may not be administered to a variety of patients.

Chronic obstructive pulmonary disease (COPD) is characterized by airflow obstruction that is generally caused by chronic bronchitis, emphysema, or both. Emphysema is characterized by abnormal permanent enlargement of the air spaces distal to the terminal bronchioles, accompanied by destruction of their walls and without obvious fibrosis. Chronic bronchitis is characterized by chronic cough, mucus production, or both, for at least three months for at least two successive years where other causes of chronic cough have been excluded. COPD characteristically affects middle aged and elderly people, and is one of the leading causes of morbidity and mortality worldwide. In the United States it affects about 14 million people and is the fourth leading cause of death, and both its morbidity and mortality rates are still rising. This contrasts with the decline over the same period in age-adjusted mortality from all causes, and from cardiovascular diseases. COPD, however, is preventable, since it is believed that its main cause is exposure to cigarette smoke. The disease is rare in lifetime non-smokers, in whom exposure to environmental tobacco smoke will explain at least some of the airways obstruction. Other proposed etiological factors include airway hyper-responsiveness or hypersensitivity, ambient air pollution, and allergy. The airflow obstruction in COPD is usually progressive in people who continue to smoke. This results in early disability and shortened survival time. Stopping smoking reverts the decline in lung function to values for non-smokers. Many patients will use medication chronically for the rest of their lives, with the need for increased doses and additional drugs during exacerbations. Amongst the currently available treatments for COPD, short-term benefits were found, as opposed to long term effects on progression, from anti-cholinergic drugs, β2 adrenergic agonists, and oral steroids. The effects of anti-cholinergic drugs and β2 adrenergic agonists, however, are not seen in all people with COPD, and the two agents combined are only slightly more effective than either alone. Their adverse effects and the need for frequent monitoring of blood concentrations limit the usefulness of theophyllines. There is no evidence that anti-cholinergic agents affect the decline in lung function, and mucolytics have been shown to reduce the frequency of exacerbations but with a possible deleterious effect on lung function. The long-term effects of β2 adrenergic agonists, oral corticosteroids, and antibiotics have not yet been evaluated, and up to the present time no other drug has been shown to affect the progression of the disease or survival. Thus, there is very little currently available to alleviate symptoms of COPD, prevent exacerbations, preserve optimal lung function, and improve daily living activities an quality of life. Thus, there is very little currently available to alleviate symptoms of COPD, prevent exacerbations, preserve optimal lung function, and improve daily living activities an quality of life.

Interstitial lung disease (ILD), interstitial pulmonary fibrosis, or simply pulmonary fibrosis are terms that include more than 130 chronic lung disorders that affect the lung in at least three ways: lung tissue is damaged in some known or unknown way, walls of the air sacs in the lung become inflamed, and scarring or fibrosis begins in the interstitium (or tissue between the air sacs), and the lung becomes stiff. Breathlessness during exercise may be one of the first symptoms of these diseases, and a dry cough may be present. Neither the symptoms nor X rays are often sufficient to tell apart different types of pulmonary fibrosis. Some pulmonary fibrosis patients have known causes and some have unknown or idiopathic causes. Interstitial lung disease (or pulmonary fibrosis) is named after he tissue between the air sacs of the lungs because this is the tissue affected by fibrosis or scarring. The course of this disease is generally unpredictable. If they progress the lung tissue thickens and becomes stiff, breathing becomes more difficult and demanding, and inflammation occurs. Some people may need oxygen therapy as part of their treatment.

Microbial infections are extremely common, and may be caused by viruses, bacteria, and other forms of life. They are generally treated with anti-viral agents, antibiotics, and other specific therapeutic drugs. However, some infections may either go unnoticed, or produce secondary effects such as inflammation, pulmonary and airway obstructions, and other pulmonary ailments.

Cancer is one of the most prevalent and feared diseases of our times. It generally results from the carcinogenic transformation of normal cells of different epithelia. Two of the most damaging characteristics of carcinomas and other types of malignancies are their uncontrolled growth and their ability to create metastases in distant sites of the host particularly a human host. It is usually these distant metastases that cause serious consequences to the host since frequently the primary carcinoma may be, in most cases, removed by surgery. The treatment of cancer presently relies on surgery, irradiation therapy and systemic therapies such as chemotherapy, different immunity-boosting medicines and procedures, hyperthermia and systemic, radioactively labeled monoclonal antibody treatment immunotoxins and chemotherapeutic drugs.

Adenosine may constitute an important mediator in the lung for various diseases, including bronchial asthma, COPD, CF, RDS, rhinitis, pulmonary fibrosis, and others. Its potential role was suggested by the finding that asthmatics respond favorably to aerosolized adenosine with marked bronchoconstriction whereas normal individuals do not. An asthmatic rabbit animal model, the dust mite allergic rabbit model for human asthma, responded in a similar fashion to aerosolized adenosine with marked bronchoconstriction whereas non-asthmatic rabbits showed no response. More recent work with this animal model suggested that adenosine-induced bronchoconstriction and bronchial hyperresponsiveness in asthma may be mediated primarily through the stimulation of adenosine receptors. Adenosine has also been shown to cause adverse effects, including death, when administered therapeutically for other diseases and conditions in subjects with previously undiagnosed hyper reactive airways.

Adenosine is a purine involved in intermediary metabolism, and may constitute an important natural mediator of many of diseases. Adenosine plays a unique role in the body as a regulator of cellular metabolism. It can raise the cellular level of AMP, ADP and ATP which are the energy intermediates of the cell. Adenosine can stimulate or down regulate the activity of adenylate cyclase and hence regulate cAMP levels. cAMP, in turn, plays a role in neurotransmitter release, cellular division and hormone release. Adenosine's major role appears to be to act as a protective injury autocoid. In any condition in which ischemia, low oxygen tension or trauma occurs adenosine appears to play a role. Defects in synthesis, release, action and/or degradation of adenosine have been postulated to contribute to the over activity of the brain excitatory amino acid neurotransmitters, and hence various pathological states. Adenosine has also been implicated as a primary determinant underlying the symptoms of bronchial asthma and other respiratory diseases, the induction of bronchoconstriction and the contraction of airway smooth muscle. Moreover, adenosine causes bronchoconstriction in asthmatics but not in non-asthmatics. Other data suggest the possibility that adenosine receptors may also be involved in allergic and inflammatory responses by reducing the hyperactivity of the central dopaminergic system. It has been postulated that the modulation of signal transduction at the surface of inflammatory cells influences acute inflammation. Adenosine is said to inhibit the production of super-oxide by stimulated neutrophils. Recent evidence suggests that adenosine may also play a protective role in stroke, CNS trauma, epilepsy, ischemic heart disease, coronary by-pass, radiation exposure and inflammation. Overall, adenosine appears to regulate cellular metabolism through ATP, to act as a carrier for methionine, to decrease cellular oxygen demand and to protect cells from ischemic injury. Adenosine is a tissue hormone or inter-cellular messenger that is released when cells are subject to ischemia, hypoxia, cellular stress, and increased workload, and or when the demand for ATP exceeds its supply. Adenosine is a purine and its formation is directly linked to ATP catabolism. It appears to modulate an array of physiological processes including vascular tone, hormone action, neural function, platelet aggregation and lymphocyte differentiation. It also may play a role in DNA formation, ATP biosynthesis and general intermediary metabolism. It is suggested that it regulates the formation of cAMP in the brain and in a variety of peripheral tissues. Adenosine regulates cAMP formation through two receptors A₁ and A₂. Via A₁ receptors, adenosine reduces adenylate cyclase activity, while it stimulates adenylate cyclase at A₂ receptors. The adenosine A₁ receptors are more sensitive to adenosine than the A₂ receptors. The CNS effects of adenosine are generally believed to be A₁-receptor mediated, where as the peripheral effects such as hypotension, bradycardia, are said to be A₂ receptor mediated.

Anti-sense oligonucleotides have received considerable theoretical consideration as potential useful pharmacological agents in human disease. One important impediment to their effective application has been a difficulty in finding an appropriate route of administration to deliver them to their site of action. The administering of anti-sense oligonucleotides directly to specific regions of the brain, for example, necessarily has limited clinical utility due to its invasive nature. Finding practical and effective applications for these agents in actual models of human disease have been few and far between, particularly because they had to be administered in large doses. The systemic administration of anti-sense oligonucleotides as pharmacological agents, such as oral and parenteral administration, has been found to have also significant problems, including the inherent difficulty in targeting specific tissues due to their dilution in the circulatory system. The bioavailability of orally administered anti-sense oligonucleotides is very low, of the order of less than about 5%. The present inventor previously pioneered the administration of oligonucleotides via the respiratory system, and successfully treated asthma, bronchoconstriction and lung inflammation and allergies, and applied the technology to the treatment of other conditions. The route of administration, thus was found to be of importance, particularly for treating localized conditions. As described in more detail below, the lung is an excellent target for the direct administration of anti-sense oligonucleotides and provides a non-invasive and a tissue-specific route. The respiratory system, and in particular the lung, as the ultimate port of entry into the organism provides an excellent route of administration for anti-sense oligonucleotides. This is so not only for the treatment of lung disease, but also when utilizing the lung as a means for delivery, particularly because of its non-invasive and tissue-specific nature. Thus, local delivery of anti-sense oligos directly to the target tissue enables an optimal delivery for the therapeutic use of these compounds. Fomivirsen (ISIS 2922) is an example of a local drug delivery into the eye to treat cytomegalovirus (CMV) retinitis, for which a new drug application has been filed by ISIS. The administration of a drug through the lung offers the further advantage that inhalation is non-invasive whereas direct injection into the vitreous of the eye is invasive.

Steroids are naturally occurring compounds of varied activities. In mammals, they serve different functions, some being associated with sexual cycles and reproduction, others with regulation of endogenous levels of various compounds. Some of these have anti-inflammatory activity,

Steroid hormones are potent chemical messengers that exert dramatic effects on cell differentiation, homeostasis, and morphogenesis. These molecules diverse in structure share a mechanistically similar mode of action. The effector molecules diffuse across cellular membranes and bind to specific high affinity receptors in the target cell nuclei. This interaction results in the conversion of an inactive receptor to one that can interact with the regulatory regions of target genes and modulate the rate of transcription of specific gene sets. Upon ligand binding, these receptors generate both rapid and long lasting responses. Steroids can act through two basic mechanisms: genomic and non-genomic. The classical genomic action is mediated by specific intracellular receptors, whereas the primary target for the non-genomic one is the cell membrane. Many clinical symptoms seem to be mediated through the non-genomic route. Furthermore, membrane effects of steroid and other factors can interfere with the intranuclear receptor system inducing or repressing steroid-and receptor-specific genomic effects. These signalling pathways may lead to unexpected hormonal or anti-hormonal effects in patients treated with certain drugs.

Steroid receptors are members of a large family of nuclear transcription factors that regulate gene expression by binding to their cognate steroid ligands, to the specific enhancer sequences of DNA (steroid response elements) and to the basic transcription machinery. Steroid receptors are basically localized in the nucleus, regardless of hormonal status, and considerable-amounts of unliganded steroid receptors may be present in the cytoplasm of target cells in exceptional cases Most steroid receptors are phosphoproteins, which are further phosphorylated after ligand binding. The role of phosphorylation in receptor transaction is complex and may not be uniform to all steroid receptors. However, pliosphorylation and/or dephosphorylation is believed to be a key event regulating the transcriptional activity of steroid receptors. Steroid receptor activities can be affected by the amount of steroid receptor in the cell nuclei, which is modified by the rate of transcription and translation of the steroid receptor gene as well as by proteolysis of the steroid receptor protein. There is an auto- and heteroregulation of receptor levels. Some of the steroid receptors appear to bind specific protease inhibitors and exhibit protease activity. Some steroid receptors are expressed as two or more isoforms, which may have different effects on transcription. Receptor isoforms are different translation or transcription products of a single gene. Isoform A of the progesterone receptor is a truncated form of PR isoform B originating from the same gene, but it is able to suppress not only the gene enhancing activity of PR-B but also that of other steroid receptors.

Before hormone binding, the receptors are part of a complex with multiple chaperones which maintain the receptor in its steroid binding conformation. Following hormone binding, the complex dissociates and the receptors bind to steroid response elements in chromatin. Regulation of gene expression by hormones involves an interaction of the DNA-bound receptors with other sequence-specific transcription factors and with the general transcription factors, which is partly mediated by co-activators and co-repressors. The specific array of cis regulatory elements in a particular promoter/enhancer region, as well as the organization of the DNA sequences in nucleosomes, specifies the network of receptor interactions. Depending on the nature of these interactions, the final outcome can be induction or repression of transcription.

Adrenocortical hormones are steroid hormones classified as glucocorticoids, mineralocorticoids and sex hormones. Glucocorticoids moderate the metabolism of sugar, fat and protein and may raise the resistance to the adverse stimulation of the body by these substances. Many of the clinically useful steroids belong to this group, including cortisone, hydrocortisone, and their pharmaceutical derivatives such as prednisone, dexamethasone, etc. Although glucocorticoids were originally so called because of their infuence on glucose metabolism, they are currently defined as steroids that exert their effects by binding to specific cytosolic receptors that mediate the actions of these hormones. These glucocorticoid receptors are present in virtually all tissues, and glucocorticoid-receptor interactions are responsible for most of the known effects of these steroids. Alteration in the structure of these glucocorticoids has led to the development of synthetic compounds with greater glucocorticoid activity. The increased activity of these compounds is due to increased affinity for the glucocorticoid receptors and/or delayed plasma clearance, which increases tissue exposure. In addition, many of these synthetic glucocorticoids evidence negligible mineralocortocoid effects and thus do not result in sodium retention, hypertension, and/or hypokalemia. Glucocorticoid action is initiated by entry of the steroid into the cell and binding to the cytosolic glucocorticoid receptor proteins. After binding, activated hormone-receptor complexes enter the nucleus and interact with nuclear chromatin acceptor sites. These events cause the expression of specific genes and the transcription of specific mRNAs. The resulting proteins affect the response to the glucocorticoids, which may be inhibitory or stimulatory depending on the specific tissue affected. Although glucocorticoid receptors are similar in many tissues, the proteins synthesized vary widely and are the result of expression of specific genes in different cell types.

Mineralocorticoids and sex hormones are non-glucocorticoid steroids, e.g., adrenal androgens. Adrenal androgens, such as androstenediones, dehydroepiandrosterone (DHEA), and DHEA sulfate function as precursors for the peripheral conversion to androgenic hormones, such as testosterone and dihydrotestosterone. DHEA sulfate secreted by the adrenal undergoes limited conversion to DHEA, and both the peripheral DHEA and DHEA secreted by the adrenal cortex may be further converted in peripheral tissues to androstenedione, the immediate precursor of the active androgens. Dehydroepiandrosterone (DHEA) is a naturally occurring steroid secreted by the adrenal cortex with apparent chemoprotective properties. Epidemiological studies have shown that low endogenous levels of DHEA correlate with increased risk of developing some forms of cancer, such as pre-menopausal breast cancer in women and bladder cancer in both sexes. The ability of DHEA and DHEA analogues, e.g. dehydroepiandrosterone sulfate (DHEA-S), to inhibit carcinogenesis is believed to result from their uncompetitive inhibition of the activity of the enzyme glucose 6-phosphate dehydrogenase (G6PDH). G6PDH is the rate limiting enzyme of the hexose monophosphate pathway, a major source of intracellular ribose-5-phosphate and NADPH. Ribose-5 phosphate is a necessary substrate for the synthesis of both ribo- and deoxyribonucleotides required for the synthesis of RNA and DNA. NADPH is a cofactor also involved in nucleic acid biosynthesis and the synthesis of hydroxmethylglutaryl Coenzyme A reductase (HMG CoA reductase). HMG CoA reductase is an unusual enzyme that requires two moles of NADPH for each mole of product, mevalonate, produced. Thus, it appears that HMG CoA reductase would be ultrasensitive to DHEA-mediated NADPH depletion, and that DHEA-treated cells would rapidly show the depletion of intracellular pools of mevalonate. Mevalonate is required for DNA synthesis, and DHEA arrests human cells in the G1 phase of the cell cycle in a manner closely resembling that of the direct HMG CoA. Because G6PDH produces mevalonic acid used in cellular processes such as protein isoprenylation and the synthesis of dolichol, a precursor for glycoprotein biosynthesis, DHEA inhibits carcinogenesis by depleting mevalonic acid and thereby inhibiting protein isoprenylation and glycoprotein synthesis. Mevalonate is a central precursor for the synthesis of cholesterol, as well as for the synthesis of a variety of non-sterol compounds involved in post-translational modification of proteins, such as farnesyl pyrophosphate and geranyl pyrophosphate. Mevalonate is also a central precursor for the synthesis of dolichol, a compound that is required for the synthesis of glycoproteins involved in cell-to-cell communication and cell structure. Mevalonate is also central to the manufacture of ubiquinone, an anti-oxidant with an established role in cellular respiration. It has long been known that patients receiving steroid hormones of adrenocortical origin at pharmacologically appropriate doses show increased incidence of infectious disease.

DHEA, also known as 3β-hydroxyandrost-5-en-17-one or dehydroepiandrosterone, is a 17-ketosteroid which is quantitatively one of the major adrenocortical steroid hormones found in mammals. Although DHEA appears to serve as an intermediary in gonadal steroid synthesis, the primary physiological function of DHEA has not been fully understood. It has been known, however, that levels of this hormone begin to decline in the second decade of life, reaching 5% of the original level in the elderly.) Clinically, DHEA has been used systemically and/or topically for treating patients suffering from psoriasis, gout, hyperlipemia, and it has been administered to post-coronary patients. In mammals, DHEA has been shown to have weight optimizing and anti-carcinogenic effects, and it has been used clinically in Europe in conjunction with estrogen as an agent to reverse menopausal symptoms and also has been used in the treatment of manic depression, schizophrenia, and Alzheimer's disease. DHEA has also been used clinically at 40 mg/kg/day in the treatment of advanced cancer and multiple sclerosis. Mild androgenic effects, hirsutism, and increased libido were the side effects observed. These side effects can be overcome by monitoring the dose and/or by using analogues. The subcutaneous or oral administration of DHEA to improve the hosts response to infections is known, as is the use of a patch to deliver DHEA. DHEA is also known as a precursor in a metabolic pathway that ultimately leads to more powerful agents that increase immune response in mammals. That is, DHEA acts as a biphasic compound: it acts as an immuno-modulator when converted to androstenediol or androst-5-ene-3β,17β-diol (βAED), or androstenetriol or androst-5-ene-3β,7β,17β-triol (βAET). However, in vitro DHEA has certain lymphotoxic and suppressive effects on cell proliferation prior to its conversion to βAED and/or βAET. It is, therefore, believed that the superior immunity enhancing properties obtained by administration of DHEA result from its conversion to more active metabolites.

Adequate ubiquinone levels have been found to be essential for maintaining proper cardiac function, and the administration of exogenous ubiquinone has recently been shown to have beneficial effect in patients with chronic heart failure. Ubiquinone depletion has been observed in humans and animals treated with lovastatin, a direct HMG CoA reductase inhibitor. Such lovastatin-induced depletion of ubiquinone has been shown to lead to chronic heart failure, or to a shift from low heart failure into life-threatening high grade heart failure. DHEA, unlike lovastatin, inhibits HMG CoA reductase indirectly by inhibiting G6PDH and depleting NADPH, a required cofactor for HMG CoA reductase. However, DBEA's indirect inhibition of HMG CoA reductase suffices to deplete intracellular mevalonate. This effect adds to the depletion of ubiquinone, and may result in chronic heart failure following long term usage. Thus, although DHEA was once considered a safe drug, it is now predicted that with long term administration of DHEA or its analogues, chronic heart failure may occurs as a complicating side effect. Further, some analogues of DHEA produce this side effect to a greater extent because, in general, they are more potent inhibitors of G6PDH than DHEA.

A handful of medicaments have been used for the treatment of respiratory diseases and conditions, although in general they all have limitations. Amongst them are corticoid steroids with glucocorticoid activity, leukotriene inhibitors, anti-cholinergic agents, anti-histamines, oxygen therapy, theophyllines, and mucolytics. Corticosteroids are the ones with the most widespread use in spite of their well documented side effects. Most of the available drugs are nevertheless effective in a small number of cases, and not at all when it comes to the treatment of asthma. No treatments are currently available for many of the other respiratory diseases. Theophylline, an important drug in the treatment of asthma, is a known adenosine receptor antagonist that was reported to eliminate adenosine-mediated bronchoconstriction in asthmatic rabbits. A selective adenosine A₁ receptor antagonist, 8-cyclopentyl-1,3-dipropylxanthine (DPCPX) was also reported to inhibit adenosine-mediated bronchoconstriction and bronchial hyperresponsiveness in allergic rabbits. The therapeutic and preventative applications of currently available adenosine A₁ receptor-specific antagonists are, nevertheless, limited by their toxicity. Theophylline, for example, has been widely used in the treatment of asthma, but is associated with frequent, significant toxicity resulting from its narrow therapeutic dose range. DPCPX is far too toxic to be useful clinically. The fact that, despite decades of extensive research, no specific adenosine receptor antagonist is available for clinical use attests to the general toxicity of these agents.

For many years, two classes of compounds have dominated the treatment of asthma: corticosteroids having glucocorticoid activity and bronchodilators. Examples of corticosteroids are beclomethasone and corticoid 21-sulfopropionates. Examples of a bronchodilator are an older β2 adrenergic agonist such as albuterol, and a newer one such as salmeterol. In general, when glucocorticosteroids are taken daily either by inhalation or orally, they attenuate inflammation. The β2 adrenergic agonists, on the other hand, primarily alleviate bronchoconstriction. Whereas glucocorticosteroids are not useful in general for acute settings, bronchodilators are used in acute care, such as in the case of asthma attacks. At the present time, many asthma patients require daily use of both types of agents, a glucocorticosteroid to contain pulmonary inflammation, and a bronchodilator to alleviate bronchoconstriction. More recently, fluticasone propionate, a corticosteroid was combined with β2 adrenergic agonists in one therapeutic formulation said to have greater efficiency in the treatment of asthma. However, glucocorticosteriods, particularly when taken for prolonged periods, have extremely deleterious side effects that, although somewhat effective, make their chronic use undesirable, particularly in children.

Clearly, there exists a well defined need for novel and effective therapies for treating respiratory, lung and cancer ailments that cannot presently be reasonably treated, or at least for which no therapies are available that are effective and devoid of significant detrimental side effects. Moreover, there is a definite need for treatments that have prophylactic and therapeutic applications, and require low amounts of active agents, and are less costly and less prone to detrimental side effects. Furthermore, it is readily apparent that anti-inflammatory steroids (“AIS”), including adrenal androgens, androgens and their derivatives, etc, corticoid and non-glucocorticoid steroids, ubiquinones and their respective salts, as well as specifically targeted anti-sense oligonucleotides (oligos) are each alone useful for the treatment of respiratory, lung, and cancer. This patent provides their joint effects that evidence unexpected superior results over each agent alone.

### SUMMARY OF THE INVENTION

The present invention generally relates to a pharmaceutical or veterinary composition, comprising a pharmaceutically or veterinarily acceptable carrier or diluent, and first and second active agents.

The first active agent comprises an oligonucleotide(s) (oligo(s)) that may be anti-sense to one or more targets, and a second active agent comprising anti-inflammatory steroids (“AIS”) and/or a ubiquinone, in amounts effective for alleviating airway, lung, and microbial and/or cancer diseases associated with, for example, bronchoconstriction, impeded respiration, dispnea, emphysema, asthma, COPD, ARDS, CF, allergic rhinitis, pulmonary hypertension and fibrosis, lung inflammation, allergies, surfactant depletion or hyposecretion, and cancers, among others. The oligo preferably contains about 0 to about 15% adenosine (A) and is anti-sense to the initiation codon, the coding region, the 5′-end or the 3′-end genomic flanking regions, the 5′ or 3′ intron-exon junctions, or regions within 2 to 10 nucleotides of the junctions of at least one gene regulating or encoding a target polypeptide associated with lung or airway dysfunction or cancer, or that is anti-sense to the corresponding mRNA, and the composition may comprise also combinations or mixtures of the oligos. The targets are typically molecules associated with airway disease, cancer, etc., such as transcription factors, stimulating and activating peptide factors, cytokines, cytokine receptors, chemokines, chemokine receptors, adenosine receptors, bradykinin receptors, endogenously produced specific and non-specific enzymes, immunoglobulins and antibodies, antibody receptors, central nervous system (CNS) and peripheral nervous and non-nervous system receptors, CNS and peripheral nervous and non-nervous system peptide transmitters, adhesion molecules, defensins, growth factors, vasoactive peptides and receptors, binding proteins, and malignancy associated proteins, among others. In one embodiment the first active agent comprises a nucleic acid wherein the oligo is anti-sense to more than one target. These are called within the four corners of this patent multiple target anti-sense oligonucleotides or MTAS.

The second active agent comprises an anti-inflammatory steroid such as an adrenal androgen of the chemical formula 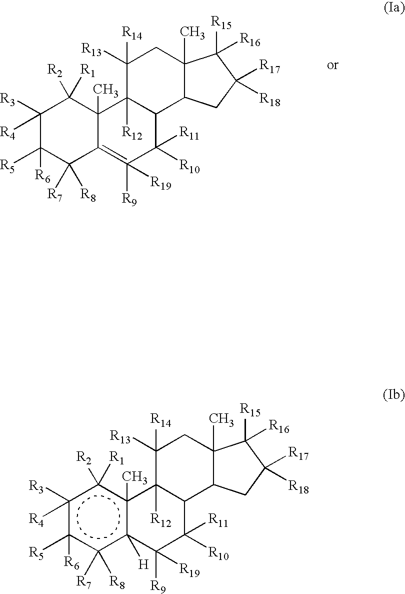
wherein R₁, R₂, R₃, R₄, R₆, R₇, R₈, R₉, R₁₀, R₁₂, R₁₃, R₁₄ and R₁₉ are independently H, OR, halogen, (C₁-C₁₀) alkyl, (C₁-C₁₀) alkene, (C₁-C₁₀) alkyne, (C₁-C₁₀) alkoxy, or two or more of R₁, R₂, R₃, R₄, R₆, R₇, R₈, R₉, R₁₀, R₁₂, R₁₃, R₁₄ and R₁₉ can be linked by combination of the atoms of C, O, N, S, P and Si to form a 3 to 15 member ring(s), in the α- and/or β-configuration;
R₅, R₆, R₁₀, and R₁₁ are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R₂₀, —OPOR₂₀R₂₁, (C₁-C₁₀) alkyl, (C₁-C₁₀) alkene, (C₁-C₁₀) alkyne or OR₂₃, wherein, R₂₃ is hydrogen or SO₂OM, wherein M is selected from H, Na, sulfatide; 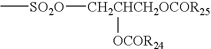
or phosphatide 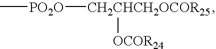
wherein R₂₄ and R₂₅, which may be the same or different, are straight or branched (C₁-C₂₀) alkyl, (C₁-C₂₀) alkene, (C₁-C₂₀) alkyne, sugar, polyethyleneglycol (PEG) or glucuronide 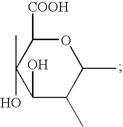
R₅ and R₆ taken together are ═O;
R₁₀ and R₁₁, taken together are ═O;
R₁₅ is (1) H, halogen, (C₁-C₁₀) alkyl, (C₁-C₁₀) alkene, (C₁-C₁₀) alkyne, or (C₁-C₁₀) alkoxy when R₁₆ is —C(O)OR₂₂, (2) H, halogen, OH, (C₁-C₁₀) alkyl, (C₁-C₁₀) alkene or (C₁-C₁₀) alkyne, when R₁₆ is halogen, OH, (C₁-C₁₀) alkyl, (C₁-C₁₀) alkene or (C₁-C₁₀) alkyne, (3) H, halogen, (C₁-C₁₀) alkyl(C₁-C₁₀) alkenyl, (C₁-C10) alkynyl, formyl, (C₁-C₁₀) alkanoyl or epoxy when R₁₆ is OH, (4) OR, SR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R₂₀ or —OPOR₂₀R₂₁ when R₁₆ is H, or R₁₅ and R₁₆ taken together are ═O;
R₁₇ and R₁₈ is are independently (1) H, —OH, halogen, (C₁-C₁₀) alkyl, (C₁-C₁₀) alkene, (C₁-C₁₀) alkyne or —(C₁-C₁₀) alkoxy when R₆ is H OR, halogen, (C₁-C₁₀) alkyl or —C(O)OR₂₂, (2) H, (C₁-C₁₀alkyl)ₙ amino, (C₁-C₁₀ alkene)ₙ amino, (C₁-C₁₀ alkyne)ₙ amino, ((C₁-C₁₀)alkyl)ₙ amino-(C₁-C₁₀) alkyl, ((C₁-C₁₀)alkene)ₙ amino-C₁-C₁₀)alkyl, ((C₁-C₁₀) alkyne)ₙ amino-(C₁-C₁₀) alkyl, ((C₁-C₁₀)alkyl)ₙ amino-(C₁-C₁₀) alkene, ((C₁-C₁₀)alkene)ₙ amino-(C₁-C₁₀) alkene, ((C₁-C₁₀)alkyne)ₙ amino-(C₁-C₁₀)alkene, ((C₁-C₁₀)alkyl)ₙ amino-(C₁-C₁₀)alkyne, ((C₁-C₁₀) alkene)ₙ amino-(C₁-C₁₀) alkyne, ((C₁-C₁₀)alkyne)ₙ amino-(C₁-C₁₀)alkyne, (C₁-C₁₀)alkoxy, hydroxy-(C₁-C₁₀)alkyl, hydroxy-(C₁-C₁₀) alkene, hydroxy-(C₁-C₁₀)alkyne, (C₁-C₁₀)alkoxy-(C₁-C₁₀)alkyl, (C₁-C₁₀) alkoxy-(C₁-C₁₀) alkene, (C₁-C₁₀) alkoxy-(C₁-C₁₀) alkyne, (halogen)ₘ (C₁-C₁₀) alkyl, (halogen)ₘ (C₁-C₁₀) alkene, (halogen)ₘ (C₁-C₁₀) alkyne, (C₁-C₁₀) alkanoyl, formyl, (C₁-C₁₀) carbalkoxy or (C₁-C₁₀) alkanoyloxy when R₁₅ and R₁₆ taken together are ═O, (3) R₁₇ and R₁₈ taken together are ═O; (4) R₁₇ and R₁₈ is taken together with the carbon to which they are attached form a 3-6 member ring containing 0 or 1 oxygen atom; or (5) R₁₅ and R₁₇ taken together with the carbons to which they are attached form an epoxide ring; R₂₀ and R₂₁ are independently OH, pharmaceutically acceptable ester or pharmaceutically acceptable ether; R₂₂ is H, (halogen)ₘ (C₁-C₁₀) alkyl, (halogen)ₘ (C₁-C₁₀) alene, (halogen)ₘ (C₁-C₁₀) alkyne, (C₁-C₁₀) alkyl, (C₁-C₁₀) alkene or (C₁-C₁₀) alkyne; n is 0, 1 or 2; and m is 1, 2 or 3,
or pharmaceutically or veterinarily acceptable salts thereof; and/or
a ubiquinone of the chemical formula 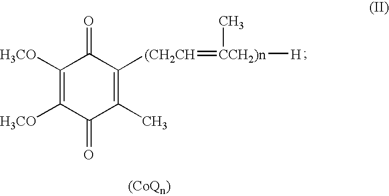
   wherein n=1 to 12, the agent being present in an amount effective for treating respiratory lung diseases and conditions, or for reducing levels of, or sensitivity to, adenosine or for increasing surfactant or ubiquinone levels in a subject's tissue (s), or pharmaceutically acceptable salts thereof.

The oligos and the anti-inflamatory steroids (“AIS”) and/or ubiquinones (the second agent) are provided in the form of separate compositions and formulations together with a carrier or diluent, and optionally with other therapeutic agents and formulation additives. The first and second active agents are also provided as a single composition in combination with a carrier and other ingredients known in the art, and may be provided jointly or separately contained in a capsule or cartridge, and in the form of a kit. The drawings accompanying this patent form part of the disclosure of the invention, and further illustrate some aspects of the present invention as discussed below.

### BRIEF DESCRIPTION OF THE DRAWINGS

FIG. 1 illustrates the inhibition of HT-29 SF cells by DHEA.

FIGS. 2A and 2B illustrate the effects of different amounts of DHEA on cell cycle distribution in HT-29 SF cells.

FIGS. 3A and 3B illustrate the reversal of DHEA-induced growth inhibition in HT-29 cells treated with CON: Control; MVA: Mevalonic Acid; SQ: Squaline; CH: Cholesterol; DN: Deoxyribonucleodies; RN: Ribonucleosides.

FIGS. 4A, 4B, 4C and 4D illustrate the reversal of DHEA-induced G1 arrest in HT-29 SF cells for different durations of treatment with DHEA.

The invention will now be described in general in conceptual and experimental terms, with reference to specific examples. Other objects, advantages and features of the present invention will become apparent to those skilled in the art from the description that follows.

### DETAILED DESCRIPTION OF THE PREFERRED EMBODIMENTS

This invention arose from a desire by the inventor to improve on his own prior treatments and those of others for diseases of the respiratory and pulmonary tracts, as well as those that develop elsewhere in the mammalian body. While he previously provided a pioneering treatment for respiratory tract conditions employing oligonucleotide anti-sense to pre-selected targets, and a treatment for respiratory conditions employing dehydroepiandrosterones and ubiquinone, he reasoned further that their combination might produce unexpectedly superior results given their independent mechanisms. Moreover, he posited that the combination of low dose anti-sense oligonucleotide (oligo) therapy with steroids in general and/or ubiquinone therapy would afford the advantage of their independent lack of detrimental side effects when compared with other agents such as steroids alone, and many others that are generally fraught with detrimental side effects and by the need of administering high doses of therapeutical agents. The inventor's prior discovery that variously targeted anti-sense oligonucleotides (oligos) may be utilized therapeutically in the treatment of diseases or conditions which impair respiration, cause inflammation and/or allergy(ies) in the lung and elsewhere, constrict bronchial tissue, obstruct lung airways, deplete surfactant secretion, and/or otherwise impede normal breathing, lead him to expand his work to their combination with steroids of broad classifications, whose association, either known or discovered by him, with respiratory and pulmonary diseases as well as heart, brain, kidney, skin and other conditions, e.g. ailments associated with hypoxia, infantile Respiratory Disorder Syndrome (RDS), Acute Respiratory Disorder Syndrome (ARDS), aging, cardiac disease, cardiovascular problems, asthma, respiratory distress syndrome, rhinitis, pain, cystic fibrosis (CF), pulmonary hypertension, pulmonary vasoconstriction, pulmonary fibrosis, emphysema, chronic obstructive pulmonary disease (COPD), allergic rhinitis, and cancers such as lung cancer, leukemias, lymphomas, carcinomas, and the like, including colon cancer, breast cancer, lung cancer, pancreatic cancer, hepatocellular carcinoma, kidney cancer, melanoma, etc., as well as all types of cancers which may metastasize or have metastasized to the lung(s), including breast, liver and prostate cancer, would clearly find an immediate therapeutic application. In general, many diseases and conditions are associated with or cause inflammation, constricted bronchial tissue or lung airways, depletion of surfactant secretion, or augmented respiratory tract allergy(ies), or otherwise impede normal breathing.

The present treatment employs two agents, the first agent being selective for specific targets associated with or mediating these symptoms, and when administered into the airways it is employed in doses up to 1000-fold lower than previously seen in the art. The other agent includes a steroid agent and/or a ubiquinone and provides a more generalized amelioration of the symptoms, also in the substantial absence of undesirable side effects. This treatment further improves on the inventor's prior separate oligonucleotide (oligo) treatment by selecting oligos of reduced adenosine content, or otherwise reducing their adenosine content to reduce the release of free adenosine (A) by breakdown of A-containing oligonucleotides (oligos), thereby avoiding activating adenosine receptors that aggravate bronchoconstriction, and respiratory tract inflammation and allergies, lung surfactant depletion, and the like. As further described below, this patent also provides for the substitution of other bases with a universal base(s) (U) when some characteristic is to be modified. This patent provides novel and improved compositions, formulations, kits and methods which afford greatly improved results when compared with previously known independent treatments for preventing and alleviating bronchoconstriction, allergy(ies), inflammation, breathing difficulties, surfactant depletion and blockage of airways, as well as for preventing and alleviating other conditions and diseases which, directly or indirectly, affect the lung tissue. In different embodiments, one or more nucleic acids of the invention may be formulated for their administration alone or in combination with the steroid agents and/or ubiquinones, surfactant(s), a carrier, and/or other therapeutic agents and formulation agents known in the art Similarly, the anti-inflammatory steroids and the ubiquinones may be formulated separately for separate administration, or with various formulation components, other therapeutic agents, and the like. By means of example, the steroids and ubiqionone may be administered once or twice daily whereas the oligo may only need be administered once weekly or biweekly.

The single or multiple active agent compositions of this invention are provided in a variety of systemic and topical formulations suitable for the delivery of anti-sense oligonucleotides (oligos) and anti-inflammatory steroids and/or ubiquinones by different routes as a fast means of starting treatment to address asthma and other pulmonary and respiratory tract diseases that may have a rapid onset, where a very low drug dosage is desirable. On the other hand, the oligos have long half-lives and may be administered as preventative of acute episodes, to significantly reduce emergency visits to a doctor or hospital, and as prophylactic maintenance treatment due to the high tolerability of the active agents for prolonged periods of time. In one embodiment, the present treatment provides a once-a-week oligo therapy, accompanied by daily administration of ubiquinone and/or a steroid incorporated into a subject's daily routine. This regime may be effectively administered preventatively, prophylactically and therapeutically, in conjunction with other therapies, or by itself for conditions without known therapies or as a substitute for therapies that have significant negative side effects is also of immediate clinical application. The present treatment also finds an application in the treatment of malignancies, given that steroids and ubiquinones are known for their carcinogenic activities as well as beneficial respiratory effects.

In these cases, the oligo are targeted to cancer-associated nucleic acids and their products. General examples of oligo(s) of the invention are those targeted to a receptor(s) and it (they) are typically present in the composition in an amount effective to reduce that receptor(s) mediated effect(s), and for reducing airway obstruction, lung inflammation and allergy(ies), and surfactant depletion, among others. In one embodiment the receptor is preferably an adenosine receptor such as the adenosine A₁, A_{2b}, or A₃ receptors, and in some instances even adenosine A₂ₐ receptors. The oligo of the invention may be applied to the preparation of a medicament for reducing bronchoconstriction, impeded respiration, lung inflammation and allergy(ies), depletion of surfactant or ubiquinone, and for treating respiratory and pulmonary conditions in general, and specific ones such asthma, ARDS, pulmonary fibrosis, cystic fibrosis, allergic rhinitis, COPD, etc. Many of the conditions targeted by the present treatment afflict a large segment of the population, and either remain unaddressed in terms of therapy or the existing treatments, although heavily advertised, are only mildly effective in small numbers of the afflicted population. ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. In the following paragraphs, the specific conditions will be described, and the existing treatments, if any, discussed. ARDS is currently diagnosed by mere symptomatic signs, e.g. chest auscultation with a stethoscope that may reveal abnormal symptomatic breath sounds, and confirmed with chest X-rays and the measurement of arterial blood gas. ARDS, in some instances, appears to be associated with other diseases, such as acute myelogenous leukemia, acute tumor lysis syndrome (ATLS) developed after treatment with, e.g. cytosine arabinoside, etc. In general, however, ARDS is associated with traumatic injury, severe blood infections such as sepsis or other systemic illness, high-dose radiation therapy and chemotherapy, and inflammatory responses which lead to multiple organ failure and in many cases death. In premature babies (“premies”), the lungs are not quite developed and, therefore, the fetus is in an anoxic state during development. Moreover, lung surfactant, a material critical for normal respiration, is generally not yet present in sufficient amounts at this early stage of life; however, premies often hyper-express the adenosine A₁ receptor and/or underexpress the adenosine A₂ₐ receptor and are, therefore, susceptible to respiratory problems including bronchoconstriction, lung inflammation and ARDS, among others. When Respiratory Distress Syndrome (RDS) occurs in premies, it is an extremely serious problem. Preterm infants exhibiting RDS are currently treated by ventilation and administration of oxygen and surfactant preparations. When premies survive RDS, they frequently develop bronchopulmonary dysplasia (BPD), also called chronic lung disease of early infancy, which is often fatal.

Rhinitis may be seasonal or perennial, allergic or non-allergic. Non-allergic rhinitis may be induced by infections, such as viruses, or associated with nasal polyps, as occurs in patients with aspirin idiosyncrasy. Medical conditions such as pregnancy or hypothyroidism and exposure to occupational factors or medications may cause rhinitis. The so-called NARES syndrome is a non-allergic type of rhinitis associated with eosinophils in the nasal secretions, which typically occurs in middle-age and is accompanied by some loss of sense of smell. When cholinergic pathways are stimulated they produce typical secretions that are identified by their glandular constituents so as to implicate neurologic stimulation. Other secretions typical of increased vascular permeability are found in allergic reactions as well as upper respiratory infections, and the degranulation of mast cells releases preformed mediators that interact with various cells, blood vessels, and mucous glands, to produce the typical rhinitis symptoms. Most early- and late-phase reactions occur in the nose after allergen exposure. The late-phase reaction is seen in chronic allergic rhinitis, with hypersecretion and congestion as the most prominent symptoms. When priming occurs, it exhibits a lowered threshold to stimulus after repeated allergen exposure that, in turn, causes a hypersensitivity reaction to one or more allergens. Sufferers may also become hyper-reactive to non-specific triggers such as cold air or strong odors. Saline sprays are generally used to relieve mucosal irritation or dryness associated with various nasal conditions, minimize mucosal atrophy, and dislodge encrusted or thickened mucus and are used immediately before intranasal corticosteroid dosing to prevent drug-induced local irritation. Anti-histamines such as terfenadine and astemizole, two non-sedating anti-histamines, are also employed to treat this condition, but have been associated with a ventricular arrhythmia known as Torsades de Points, usually in interaction with other medications such as ketoconazole and erythromycin, or secondary to an underlying cardiac problem Loratadine, another non-sedating anti-histamine, and cetirizine have not been associated with an adverse impact on the QT interval, or with serious adverse cardiovascular events. Cetirizine, however, produces extreme drowsiness and has not been widely prescribed. Non-sedating anti-histamines, e.g. Claritin have not been tested for asthma or other more specific conditions. Terfenadine, loratadine and astemizole, on the other hand, exhibit extremely modest bronchodilating effects, reduction of bronchial hyper-reactivity to histamine, and protection against exercise- and antigen-induced bronchospasm. Some of these benefits, however, require higher-than-currently-recommended doses. The sedating-type anti-histamines help induce night sleep, but they cause sleepiness and compromise performance if taken during the day.

When employed, anti-histamines are typically combined with a decongestant to help relieve nasal congestion. Sympathomimetic medications are used as vasoconstrictors and decongestants. The three commonly prescribed systemic decongestants, pseudoephedrine, phenylpropanolamine and phenylephrine cause hypertension, palpitations, tachycardia, restlessness, insomnia and headache. The interaction of phenylpropanolamine with caffeine, in doses of two to three cups of coffee, may significantly raise blood pressure. In addition, medications such as pseudoephedrine may cause hyperactivity in children. Topical decongestants, nevertheless, are only indicated for a limited period of time, as they are associated with a rebound nasal dilatation with overuse. Anti-cholinergic agents are given to patients with significant rhinorrhea or for specific conditions such as “gustatory rhinitis”, usually caused by ingestion of spicy foods, and may have some beneficial effects on the common cold. Cromolyn used prophylactically as a nasal spray, however, produces sneezing, transient headache, and even nasal burning. Topical corticosteroids, such as Vancenase, are somewhat effective in the treatment of rhinitis, especially for symptoms of congestion, sneezing, and runny nose. Corticosteroid nose sprays, however, sometimes, cause irritation, stinging, burning and sneezing, and sometimes local bleeding and septal perforation. The side effects of topical steroids, however, limit their usefulness except for temporary therapy in patients with severe symptoms. These agents are sometimes used for shrinking nasal polyps when local therapy fails. Immunotherapy is expensive and inconvenient, and used mostly in in-patients who experience side effects from other medications. The so-called blocking antibodies, and agents that alter cellular histamine release, in addition, decrease IgE, which is useful in IgE-mediated diseases, e.g., hypersensitivity in atopic patients with recurrent middle ear infections. For allergic rhinitis sufferers, however, a runny nose is more than a nuisance. The disorder often results in impaired quality of life and sets the stage for more serious ailments, including psychological problems. Presently, rhinitis is mostly treated with propranolol, verapamil and adenosine, all of which have Food and Drug Administration-approved labeling for acute termination of Supra Ventricular Tachycardia (SVT).

There is very little currently available to alleviate symptoms of COPD, prevent exacerbations, preserve optimal lung function, and improve daily living activities and quality of life. Anti-cholinergic drugs achieve short-term bronchodilation, but no improved long-term prognosis even with inhaled products. Most COPD patients have at least some airways obstruction, and “the lung health study” found spirometric signs of early COPD in men and women smokers. Smoking cessation produced a slowing of the decline in the functional effective volume of the lungs. While ipratropium bromide was found to have no significant effect on the decline in the functional effective volume of the patient's lungs. Ipratropium bromide, however, produced serious adverse effects, such as cardiac symptoms, hypertension, skin rashes, and urinary retention. Short and long acting inhaled β2 adrenergic agonists achieve short-term bronchodilation and provide some symptomatic relief in COPD patients, but show no meaningful maintenance effect on its progression. Short acting β2 adrenergic agonists increase exercise capacity and produce some degree of bronchodilation, and even increase lung function in some severe COPD cases. The maximum effectiveness of the newer long acting inhaled β2 adrenergic agonists was found to be comparable to that of short acting β2 adrenergic agonists. Salmeterol was found to produce modest or no change in lung function. In asthmatics, moreover, β2 adrenergic agonists have been linked to an increased risk of death, worsened control of asthma, and deterioration in lung function.

Continuous treatment of asthmatic and COPD patients with the bronchodilators ipratropium bromide or fenoterol resulted in a decline in lung function, therefore indicating that they are not suitable for maintenance treatment. The most common immediate adverse effect of β2 adrenergic agonists, however, is tremors, which at high doses may cause a fall in plasma potassium, dysrhythmias, and reduced arterial oxygen tension. The combination of a β2 adrenergic agonist with an anti-cholinergic drug provides little additional bronchodilation compared with either drug alone. Theophyllines have a small bronchodilatory effect in COPD patients but common adverse effects, such as nausea, diarrhea, headache, irritability, seizures, and cardiac arrhythmias, that occur at highly variable blood concentrations and, in many people, within the therapeutic range. In addition, they have a small therapeutic range given that blood concentrations of 15-20 mg/l are required for optimal effects. The theophylline dose must be adjusted individually based on smoking habits, infection, and other treatments, which is cumbersome. No inflammatory response to theophyllines, however, has been reported in COPD. Oral corticosteroids show some improvement in baseline functional effective volume in stable COPD patients whereas systemic corticosteroids have been found to produce some degree of osteoporosis and overt diabetes. The longer term use of oral corticosteroids may be useful in COPD, but its usefulness must be weighed against their substantial adverse effects. Inhaled corticosteroids have been found to have no significant short-term effect in airway hyper-responsiveness to histamine, but a small long-term effect on lung function, e.g., in pre-bronchodilator functional effective volume. The treatment of COPD patients with fluticasone showed a significant reduction in moderate and severe exacerbations, and a small but significant improvement in lung function and six minute walking distance. Oral prednisolone, inhaled beclomethasone or their combination had no effects in COPD patients, but lung function improved oral corticosteroids. Mucolytics have a modest effect on frequency and duration of exacerbations but an adverse effect on lung function. No mucolytics, however, have a significant effect in people with severe COPD. N-acetylcysteine, moreover, produced gastrointestinal side effects. Long-term oxygen therapy administered to hypoxaemic COPD and congestive cardiac failure patients, had little effect on death in men. In women, however, oxygen decreased the rates of death.

Although the progress and symptoms of pulmonary fibrosis and other ILDs may vary from person to person, they have one common link: they affect parts of the lung. The inflammation of the walls of the bronchioles (small airways), it is called bronchiolitis, and of the walls and air spaces of the alveoli (air sacs), it is called alveolitis. When the inflammation involves the small blood vessels (capillaries) of the lungs, it is called vasculitis. The inflammation may heal, or it may lead to permanent scarring of the lung tissue pulmonary fibrosis). This latter results in permanent loss of the tissues ability to breathe and carry oxygen, and the amount of scarring determines the level of disability a person experiences due to destruction of the air sacs and lung tissue between and surrounding the air sacs and the lung capillaries. When this happens, oxygen is generally administered to help improve breathing. Pulmonary fibrosis is generally caused by occupational and environmental exposure to irritants such as asbestos, silica and metal dusts, bacteria and animal dusts, gases and fumes, asbestosis and silicosis, infections that produce lung scarring, e.g., tuberculosis, connective or collagen tissue diseases such as Rheumatoid Arthritis, Systemic Sclerosis and Systemic Lupus Erythematosis, Idiopathic Pulmonary Fibrosis, Pulmonary Fibrosis of genetic/familial origin, and certain medicines. Many of the diseases are often named after the occupations with which they are associated, such as Grain handler's lung, Mushroom worker's lung, Bagassosis, Detergent worker's lung, Maple bark stripper's lung, Malt worker's lung, Paprika splitter's lung, and Bird breeder's lung.

“Idiopathic” (of unknown origin) pulmonary fibrosis (IPF) is the label applied when all other causes of interstitial lung disease have been ruled out, and is said to be caused by viral illness and allergic or environmental exposure (including tobacco smoke). Bacteria and other microorganisms are not thought to be a cause of IPF. There is also a familial form of the disease, known as familial idiopathic pulmonary fibrosis whose main symptom is shortness of breath. Since many lung diseases show this symptom, making a correct diagnosis is often difficult. The shortness of breath may first appear during exercise and the condition may progress then to the point where any exertion is impossible. Eventually resulting in shortness of breath even at rest. Other symptoms may include a dry cough (without sputum), and clubbing of the fingertips. Glucocorticosteroids are usually administered to treat inflammation with inconclusive results. Other drugs are added when it is clear that the steroids are in effective. Glucocorticosteroids are also used in combination with, for example, oxygen therapy in severe cases. Infection is prevented by administration of influenza and pneumococcal pneumonia vaccines. Lung biopsies are employed to assess the unpredictable response of patients to glucocorticosteroids or other immune system suppressants. Lung transplants are an ultimate option in severe cases of pulmonary fibrosis and other lung diseases. Pulmonary fibrosis may be caused by other specific diseases, such as sarcoidosis, a disease characterized by the formation of granulomas or areas of inflammatory cells, that may attack any organ of the body, most frequently the lungs, and shows enlarged lymph glands in the center of both lungs or lung tissue thickening. For many patients, sarcoidosis is a minor problem. Its symptoms including dry cough, shortness of breath, mild chest pain, fatigue, and weakness, and weight loss appears infrequently and stops even without medication. For others, it is a serious, disabling disease. Although almost everybody may develop the disease, it affects African-Americans more than members of any other race, most commonly young adults 20 to 40. Histiocytosis X, also associated with pulmonary fibrosis, seems to begin in the bronchioles or small airways of the lungs and their associated arteries and veins, and is generally followed by destruction of the bronchioles and narrowing and damaging of small blood vessels. Symptoms of this disease include a dry cough (without sputum), breathlessness upon exertion, and/or chest pain. In most cases the disease is chronic with loss of lung function, and glucocorticosteroid therapy is ineffective. Many histiocytosis X sufferers are current or former cigarette smokers mining workers, those exposed to asbestos or metal dusts or fibers, and agricultural workers exposed to particulate organic substances, such as moldy hay (Farmer's Lung). Asbestosis and silicosis are two occupational lung diseases whose causes are known. Asbestosis is caused by small needle-like particles of asbestos inhaled into the lungs that cause lung scarring or pulmonary fibrosis that may lead to lung cancer. Silicosis is a dust disease that cones from breathing in free crystalline silica dust, and is produced by all types of mining in which the ore, e.g. gold, lead, zinc, copper, iron, anthracite (hard) coal, and some bituminous (soft) coal, are extracted from quartz rock. Workers in foundries, sandstone grinding, tuuneling, sandblasting, concrete breaking, granite carving, and china manufacturing also inhaled tiny specks of silica that are carried down to the lung alveoli, where they lead to pulmonary fibrosis. There is no good therapy for this disease, but glucocorticosteroids alone, or combined drug therapy, and the hope of lung transplant are three treatments currently being tested. This patent provides the first effective therapy for these and other respiratory and lung ailments.

In the present context, the terms “adenosine, surfactant and ubiquinone depletion” are intended to encompass levels that are lowered or depleted in the subject as compared to previous levels in that subject, and levels, as well as levels in that subject but, because of some other reason, a therapeutic benefit would be achieved in the patient by modification of the levels of these agents as compared to previous levels.

The present invention, thus, provides a pharmaceutical or veterinary composition, comprising a pharmaceutically or veterinarily acceptable carrier or diluent, a first active agent comprising an anti-sense oligonucleotide(s) (oligo(s)), and a second active agent comprising an anti-inflammatory steroid and/or a ubiquinone, in amounts effective for alleviating a variety of airway or lung diseases, and other diseases such as cancers or their metastasis, among others. This invention provides the targeted administration of one or more oligo(s) in combination with a second active agent that has a more generalized effect as an anti-inflammatory, and alleviates bronchoconstriction, surfactant or ubiquinone depletion, and respiratory airway allergies. The oligos may be directed to one or more of a number of targets, and are delivered by any route, preferably through the airways to attain a fast and localized delivery through the mucosal tissue of the lungs to permit their hybridization to a desired target polynucleotide to prevent gene transcription and/or translation, thereby reducing, hampering or completely stopping gene expression. This may be attained by means of a solid powdered or liquid solution, suspension or emulsion, such as an aerosol, for administration into the respiratory airways, or direct instillation into the lung(s). While both active agents may be administered via the respiration, it is also possible to administer one by another route, e.g. steroids. The oligos employed in the composition are suitable for altering effects mediated by a variety of target polynucleic acids, such as regulatory nucleic acid sequences, genes and mRNAs, that are associated with diseases and conditions affecting the pulmonary and respiratory tracts, among others, and their associated effects, e.g. bronchoconstriction, respiratory tract inflammation, immune mediated reactions, lung surfactant deficiency(ies), respiratory allergy(ies) and other airway problems, which may be caused by different conditions, including pulmonary vasoconstriction, inflammation, respiratory allergies, asthma, impeded respiration, respiratory distress syndrome (RDS), pain, cystic fibrosis (CF), allergic rhinitis, pulmonary hypertension and fibrosis, sepsis, dispnea, acute respiratory distress syndrome (ARDS), as well as its variations in pregnant mothers and newborns (RDS), pulmonary fibrosis, emphysema, chronic obstructive pulmonary disease (COPD), bronchitis, and cancers such as leukemias, lymphomas, carcinomas, and the like, e.g. lung cancer, colon cancer, breast cancer, pancreatic cancer, hepatocellular carcinoma, kidney cancer, melanoma, hepatic metastases, etc., as well as all cancers which may metastasize or have metastasized to the lung(s), including breast and prostate cancer. The present agents are also suitable for administration before, during and after other treatments, including radiation, chemotherapy, antibody therapy, phototherapy, and cancer and other surgeries.

The second active agent is selected from an anti-inflammatory steroid such as an adrenal androgen of the chemical formula 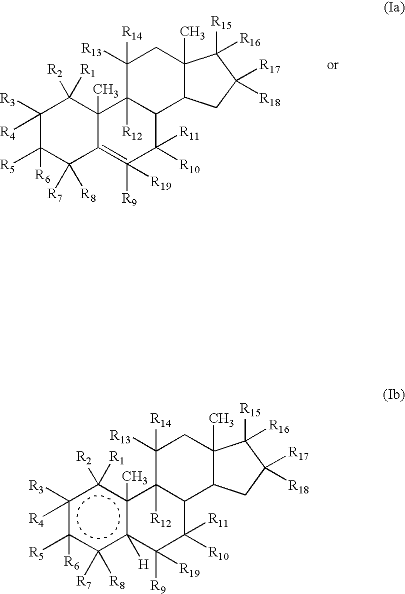
wherein R₁, R₂, R₃, R₄, R₆, R₇, R₈, R₉, R₁₀, R₁₂, R₁₃, R₁₄ and R₁₉ are independently H, OR, halogen, (C₁-C₁₀) alkyl, (C₁-C₁₀) alkene, (C₁-C₁₀) alkyne, (C₁-C₁₀) alkoxy, or two or more of R₁, R₂, R₃, R₄, R₆, R₇, R₈, R₉, R₁₀, R₁₂, R₁₃, R₁₄ and R₁₉ can be linked by combination of the atoms of C, O, N, S, P and Si to form a 3 to 15 member ring(s), in the α- and/or β-configuration;
R₅, R₆, R₁₀, and R₁₁, are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R₂₀, —OPOR₂₀R₂₁, (C₁-C₁₀) alkyl, (C₁-C₁₀) alkene, (C₁-C₁₀) alkyne or OR₂₃, wherein, R₂₃ is hydrogen or SO₂OM, wherein M is selected from H, Na, sulfatide; 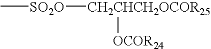
or phosphatide 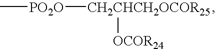
wherein R₂₄ and R₂₅, which may be the same or different, are straight or branched (C₁-C₂₀) alkyl, (C₁-C₂₀) alkene, (C₁-C₂₀) alkyne, sugar, polyethyleneglycol (PEG) or glucuronide 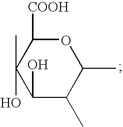
R₅ and R₆ taken together are ═O;
R₁₀ and R₁₁ taken together are ═O;
R₁₅ is (1) H, halogen, (C₁-C₁₀) alkyl, (C₁-C₁₀) alkene, (C₁-C₁₀) alkyne, or (C₁-C₁₀) alkoxy when R₁₆ is —C(O)OR₂₂, (2) H, halogen, OH, (C₁-C₁₀) alkyl, (C₁-C₁₀) alkene or (C₁-C₁₀) alkyne, when R₁₆ is halogen, OH, (C₁-C₁₀) alkyl, (C₁-C₁₀) alkene or (C₁-C₁₀) alkyne, (3) H, halogen, (C₁-C₁₀) alkyl, (C₁-C₁₀) alkenyl, (C₁-C₁₀) alkynyl, formyl, (C₁-C₁₀) alkanoyl or epoxy when R₁₆ is OH, (4) OR, SR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R₂₀ or —OPOR₂₀R₂₁ when R₁₆ is H, or R₁₅ and R₁₆ taken together are ═O;
R₁₇ and R₁₈ are independently (1) H, —OH, halogen, (C₁-C₁₀) alkyl, (C₁-C₁₀) alkene, (C₁-C₁₀) alkyne or —(C₁-C₁₀) alkoxy when R₆ is H OR, halogen, (C₁-C₁₀) alkyl or —C(O)OR₂₂, (2) H, (C₁-C₁₀alkyl)ₙ amino, (C₁-C₁₀alkene)ₙ amino, (C₁-C₁₀alkyne)ₙ amino, ((C₁-C₁₀) alkyl), amino-C₁-C₁₀) alkyl, ((C₁-C₁₀) alkene)ₙ amino-(C₁-C₁₀) alkyl, ((C₁-C₁₀) alkyne)ₙ amino-(C₁-C₁₀) alkyl, ((C₁-C₁₀) alkyl)ₙ amino-(C₁-C₁₀) alkene, ((C₁-C₁₀) alkene)ₙ amino-(C₁-C₁₀) alkene, ((C₁-C₁₀) alkyne)ₙ amino-(C₁-C₁₀) alkene, ((C₁-C₁₀) alkyl)ₙ amino-(C₁-C₁₀) alkene, ((C₁-C₁₀) alkene)ₙ amino-(C₁-C₁₀) alkyne, ((C₁-C₁₀) alkyne)ₙ amino-(C₁-C₁₀) alkyne, (C₁-C₁₀) alkoxy, hydroxy-(C₁-C₁₀) alkyl, hydroxy-(C₁-C₁₀) alkene, hydroxy-(C₁-C₁₀) alkyne, (C₁-C₁₀) alkoxy-(C₁-C₁₀) alkyl, (C₁-C₁₀) alkoxy-(C₁-C₁₀) alkene, (C₁-C₁₀) alkoxy-(C₁-C₁₀) alkyne, (halogen)ₘ (C₁-C₁₀) alkyl, (halogen)ₘ (C₁-C₁₀) alkene, (halogen)ₘ (C₁-C₁₀) alkyne, (C₁-C₁₀) alkanoyl, formyl, (C₁-C₁₀) carbalkoxy or (C₁-C₁₀) alkanoyloxy when R₁₅ and R₁₆ taken together are ═O, (3) R₁₇ and R₁₈ taken together are ═O; (4) R₁₇ and R₁₈ taken together with the carbon to which they are attached form a 3-6 member ring containing 0 or 1 oxygen atom; or (5) R₁₅ and R₁₇ taken together with the carbons to which they are attached form an epoxide ring; R₂₀ and R₂₁ are independently OH, pharmaceutically acceptable ester or pharmaceutically acceptable ether; R₂₂ is H, (halogen)ₘ (C₁-C₁₀) alkyl, (halogen)ₘ (C₁-C₁₀) alkene, (halogen)ₘ (C₁-C₁₀) alkyne, (C₁-C₁₀) alkyl, (C₁-C₁₀) alkene or (C₁-C₁₀) alkyne; n is 0, 1 or 2; and m is 1, 2 or 3; or pharmaceutically or veterinarily acceptable salts thereof; and/or
a ubiquinone of the chemical formula 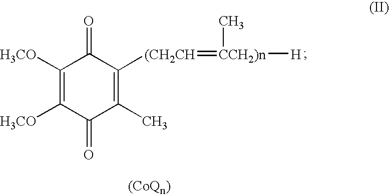
   wherein n is 1 to 12, the agent being present in an amount effective for treating respiratory lung diseases and conditions, or for reducing levels of, or sensitivity to, adenosine in a subject's tissue (s); and/or pharmaceutically acceptable salts of either of then.

One group of preferred steroids having a general formula (Ib) are 21-acetoxypregnenolone ((3β)-21-(acetyloxy)-3-hydroxypregn-5-en-20-one; Herloff and Inhoffen, U.S. Pat. No. 2,409,043); alclometasone ((7α, 11β,16α)-7-Chloro-11,17,21-trihydroxy-16-methylpregna-1,4-diene-3,20-dione; Green et al., U.S. Pat. No. 4,076,708, and Green and Shue, U.S. Pat. No. 4,124,707), or its 17,21-dipropionate form (C₂₈H₃₇ClO7); algestone ((16α)-16,17-dihydroxypregn-4-ene-3,20-dione; Colton, U.S. Pat. No. 2,727,909, Hydorn et al., U.S. Pat. No. 3,165,541, and Diassi, U.S. Pat. No. 3,027,384), its cyclic acetal with acetone form (C₂₄H₃₄O₄), or its 16α-methyl ether form (C₂₂H₃₂O₄); amcinonide ((11β,16α)-21-(acetyloxy)-16,17-[cyclopentylidenebis(oxy)]-9-fluoro-11-hydroxypregna-1,4-di-ene-3,20-dione; Shultz et al., German Patent No. 2,437,847); beclomethasone ((11β,16β)-9-chloro-11,17,21-trihydroxy-16-methylpregna-1,4-diene-3,20-dione; British Patent No. 912,378, British Patent No. 901,093, Elks et al., Belgium Patent No. 649,170, and U.S. Pat. No. 3,312,590), its dipropionate form (C₂₈H₃₇ClO₇), or its monopropionate form; betamethasone ((11β,16β)-9-fluoro-11,17,21-trihydroxy-16-methylpregna-1,4-diene-3,20-dione; U.S. Pat. No. 3,053,865, and Amiard et al., U.S. Pat. No. 3,104,246), its 21-acetate form (C₂₄H₃₁FO₆), its 21-adamantoate form (C₃₃H₄₃FO₆; Philips and English, German Patent No. 2,232,827), its 17-benzoate form (C₂₉H₃₃FO₆), its 17,21-dipropionate form (C₂₈H₃₇FO₇), its 17-valerate form (C₂₇H₃₇FO₆; Dutch Patent Application No. 6,406,615), or its 21-phospate disodium salt form (C₂₂H₂₈FNa₂O₈P); budesonide ((11β, 16α)-16,17-[butylidenebis(oxy)]-11,21-dihydropregna-1,4-diene-3,20-dione; Brattsand et al., German Patent No. 2,323,215, and U.S. Pat. No. 3,929,768); chloroprednisone ((6α)-chloro-17,21-dihydroxypregna-1,4-diene-3,11,20-trione; Batres et al., German Patent No. 1,079,042, and Ringold and Rosenkrantz, U.S. Pat. No. 2,957,895), or its 21-acetate from (C₂₃H₂₇ClO₆); ciclesonide (Taylor et al., Am J Respir Crit Care Med (1999) 160(1), 237-43); clobetasol ((11β,16β)-21-chloro-9-fluoro-11,17-dihydroxy-16-methylpregna-1,4-diene-3,20-dione; Elks et al., German Patent No. 1,902,340, and U.S. Pat. No. 3,721,687), or its 17-propionate form (C₂₅H₃₂ClFO₅); clobetasone ((16β)-21-chloro-9-fluoro-17-hydroxy-16-methylpregna-1,4-diene-3,11,20-trione; Elks et al., German Patent No. 1,902,340, and U.S. Pat. No. 3,721,687), or its 17-butyrate form (C₂₆H₃₂ClFO₅); clocortolone ((6α, 11β, 16α)-9-chloro-6-fluoro-11,21-dihydroxy-16-methylpregna-1,4-diene-3,20-dione; Dutch Patent Application No. 6,412,708, Kasper and Philippson, German Patent No. 2,011,559, and U.S. Pat. No. 3,729,495), its 21-acetate form (C₂₄H₃₀ClFO₅), or its 21-pivalate form (C₂₇H₃₆ClFO₅); cloprednol ((11β)-6-chloro-11,17,21-trihydroxypregna-1,4,6-triene-3,20-dione; France Patent No. 1,271,981, and U.S. Pat. No. 3,232,965); coroxon (phosphoric acid 3-chloro-4-methyl-2-oxo-2H-1-benzopyran-7-yl diethyl ester; Fusco et al., U.S. Pat. No. 2,951,851); cortisone (17,21-dihydroxypregn-4-ene-3,11,20-trione; Reichstein, U.S. Pat. No. 2,403,683, and Gallagher, U.S. Pat. No. 2,447,325), its 21-acetate form (C₂₃H₃₀O₆), or its 21-cyclopentanepropionate form (C₂₉H₄₀O₆), examples of brand name for cortisone include Cortone Acetate, Adreson, Altesona, Cortelan, Cortistab, Cortisyl, Cortogen, Cortone, and Scheroson; cortivazol ((11β,16α)-21-(acetyloxy)-11,17-dihydroxy-6,16-dimethyl-2′-phenyl-2′H-pregna-2,4,6-trieno[3,2-c]pyrazol-20-one; Tishler et al., U.S. Pat. No. 3,067,194, and U.S. Pat. No. 3,300,483); deflazacort ((11β, 16β)-21-(acetyloxy)-11-hydroxy-2′-methyl-5′H-pregna-1,4-dieno[17,16-d]oxazole-3,20-dione; Nathansohn and Winters, Belgium Patent No. 679,820, British Patent No. 1,077,393, and U.S. Pat. No. 3,436,389); desonide ((11β,16α)11,21-dihydroxy-16,17-[(1-methylethylidene)bis(oxy)]pregna-1,4-diene-3,20-dione; Bernstein and Allen, U.S. Pat. No. 2,990,401, Lee et al., U.S. Pat. No. 3,536,586, and Diassi and Principe, U.S. Pat. No. 3,549,498); desoximetasone ((11β,16α)-9-fluoro-11,21-dihydroxy-16-methylpregna-1,4-diene-3,20-dione; Joly et al., France Patent No. 1,296,544, U.S. Pat. No. 3,099,654, Belgium Patent No. 614,196, and Kieslich et al., U.S. Pat. No. 3,232,839); dexamethasone ((11β,16α)-9-fluoro-11,17,21-trihydroxy-16-methylpregna-1,4-diene-3,20-dione; Muller et al., U.S. Pat. No. 3,007,923, Arth et al., German Patent No. 1,113,690, and British Patent No. 869,511), its 21-acetate form (CH₂₄H₃₁FO₆), its 21-(3,3-dimethylbutyrate) form (C₂₈H₃₉FO₆; Chemerda et al., U.S. Pat. No. 2,939,873), its 21-diethylaminoacetate form (C₂₈H₄₁FNO₆), its 21-isonicotinate form (C₂₈H₄₁FNO₆), its 17,21-dipropionate form (C₂₈H₃₇FNO₆), or its 21-palmitate form (C₃₈H₅₉FO₆), examples of brand name for dexamethasone include Decadron-oral, Dexameth, Dexone, Hexadrol-oral, Dexamethasone Intensol, Dexone 0.5, Dexone 0.75, Dexone 1.5, and Dexone 4; diflorasone ((6α,11β,16β)-6,9-difluoro-11,17,21-trihydroxy-16-methylpregna-1,4-diene-3,20-dione; British Patent No. 881,334, British Patent No. 898,293, Lincoln et al., U.S. Pat. No. 3,557,158, and British Patent No. 912,015), or its diacetate form (C₂₆H₃₂F₂O₇; Ayer et al., German Patent No. 2,308,731, and U.S. Pat. No. 3,980,778); diflucortolone ((6α, 11β,16α)-6,9-difluoro-11,21-dihydroxy-16-methylpregna-1,4-diene-3,20-dione; Belgium Patent No. 639,708, and Kieslich et al., U.S. Pat. No. 3,426,128), or its 21-valerate form (C₂₇H₃₆F₂O₅); difluprednate ((6α,11β)-21-acetyloxy)-6,9-difluoro-11-hydroxy-17-(1-oxobutoxy)pregna-1,4-diene-3,20-dione; Ercoli and Gardi, South African Patent No. 680,386, and Ercoli et al., U.S. Pat. No. 3,780,177); enoxolone ((3β,20β)-3-hydroxy-11-oxoolean-12-en-29-oic acid; British Patent No. 833,184), or its 18α-hydrogen form; fluazacort ((11β,16β)-21-(acetyloxy)-9-fluoro-11-hydroxy-2′-methyl-5′H-pregna-1,4-dieno[17,16-d]oxazole-3,20-dione; British Patent No. 1,119,082, and U.S. Pat. No. 3,461,119); flucloronide ((6α,11β,16α)-9,11-dichlro-6-fluoro-21-hydroxy-16,17-[(1-methylethylidene)bis(oxy)]-pregna-1,4-diene-3,20-dione; Bowers, U.S. Pat. No. 3,201,391); flumethasone ((6α,11β,16α)-6,9-difluoro-11,17,21-trihydroxy-16-methylpregna-1,4-diene-3,20-dione; British Patent No. 902,292, and Lincoln et al., U.S. Pat. No. 3,499,016), its 21-acetate form (C₂₄H₃₀F₂O₆), or its 21-pivalate form (C₂₇H₃₆F₂O₆); fluisolide ((6α,11β,16α)-6-fluoro-11,21-dihydroxy-16,17-[(1-methylethylidene) bis(oxy)]pregna-1,4-diene-3,20-dione; British Patent No. 933,867, Ringold and Rosenkranz, U.S. Pat. No. 3,124,571, and Ringold et al., U.S. Pat. No. 3,126,375), or its 21-acetate form (C₂₆H₃₃FO₇); fluocinolone acetate ((6α,11β,16α)-6,9-difluoro-11,21-dihydroxy-16,17-[(1-methylethylidene)bis(oxy)]-pregna-1,4-diene-3,20-dione; Mills and Bowers, U.S. Pat. No. 3,014,938, and Ringold et al., U.S. Pat. No. 3,126,375); fluocinonide ((6α,11β,16α)-21-acetyloxy)-6,9-difluoro-11-hydroxy-16,17-[(1-methylethylidene)bis(oxy)]-pregna-1,4-diene-3,20-dione; British Patent No. 916,996, Ringlod and Rosenkranz, U.S. Pat. No. 3,124,571, Ringold et al., U.S. Pat. No. 3,126,375, and Fried, U.S. Pat. No. 3,197,469); fluocortin butyl((6α,11β,16α)-6-fluoro-11-hydroxy-16-methyl-3,20-dioxopregna-1,4-dien-21-oic acid butyl ester; Laurent et al., German Patent Nos. 2,150,268 and 2,150,270, and U.S. Pat. No. 3,824,260); fluocortolone ((6α,11β,16α)-6-fluoro-11,21-dihydroxy-16-methylpregna-1,4-diene-3,20-dione; Belgium Patent 614,196, and Kieslich et al., U.S. Pat. No. 3,232,839), its 21-acetate form (C₂₄H₃₁FO₅), its 21-hexanoate form (C₂₈H₃₉FO₅), or its 21-pivalate form (C₂₂H₃₇FO₅); fluorometholone ((6α,11β)-9-fluoro-11,17-dihydroxy-6-methylpregana-1,4-diene-3,20-dione; Lincoln et al., U.S. Pat. No. 2,867,637), or its 17-acetate form (C₂₄H₃₁FO₅; Magerlein et al., U.S. Pat. No. 3,038,914); fluperolone acetate([11β,17α,17(S)]-17-[2-(acetyloxy)-1-oxopropyl]-9-fluoro-11,17-dihydroxyandrosta-1,4-dien-3-one; Agnello and Laubach, U.S. Pat. No. 3,234,095); fluprednidene acetate((11β)-21-(acetyloxy)-9-fluoro-11,17-dihydroxy-16-methylenepregna-1,4-diene-3,20-dione; Wendler et al., U.S. Pat. Nos. 3,065,239, 3,068,224, 3,068,226 and 3,136,760); fluprednisolone ((6α,11β)-6-fluoro-11,17,21-trihydroxypregna-1,4-diene-3,20-dione; Batres et al., German Patent No. 1,079,042, and Lettre and Hotz, German Patent No. 1,088,953), or its 21-acetate form (C₂₃H₂₉FO₆); flurandrenolide ((6α,11β,16α)-6-fluoro-11,21-dihydroxy-16,17-[(1-methylethylidene)bis(oxy)]pregn-4-ene-3,20-dione; Ringold et al., German Patent No. 1,131,213, and U.S. Pat. No. 3,126,375); fluticasone propionate ((6α,11β,16α,17α)-6,9-difluoro-11-hydroxy-16-methyl-3-oxo-17-(1-oxopropoxy)androsta-1,4-diene-17-carbothioic acid S-(fluoromethyl) ester; Dutch Patent Application No. 8,100,707, and Philipps et al., U.S. Pat. No. 4,335,121); formocortal ((11β,16α)-21-acetyloxy)-3-(2-chloroethoxy)-9-fluoro-11-hydroxy-16,17-[(1-methylethylidene)bis(oxy)]-20-oxopregna-3,5-diene-6-carboxaldehyde; Camerino et al., France Patent No. 1,396,602, Dutch Patent Application No. 6,508,458, and U.S. Pat. No. 3,314,945); halcinonide ((11β,16α)-21-chloro-9-fluoro-11-hydroxy-16,17-[(1-methyethylidene)bis(oxy)]pregn-4-ene-3,20-dione; Difazio and Augustine, German Patent No. 2,355,710, and U.S. Pat. No. 3,892,857); halobetasol propionate (6α,11β,16β)-21-chloro-6,9-difluoro-11-hydroxy-16-methyl-17-(1-oxopropoxy)pregna-1,4-diene-3,20-dione; Kalvoda and Anner, German Patent No. 2,743,069, and U.S. Pat. No. 4,619,921); halometasone ((6α,11β,16α)-2-chloro-6,9-difluoro-11,17,21-trihydroxy-16-methylpregna-1,4-diene-3,20-dione; Anner et al., Dutch Patent Application No. 540,244, U.S. Pat. No. 3,652,554, and Swiss Patent No. 551,399), or its monohydrate form (C₂₂H₂₇ClF₂O₅.H₂O); halopredone acetate((6β,11β)-17,21-bis(acetyloxy)-2-bromo-6,9-difluoro-11-hydroxypregna-1,4-diene-3,20-dione; Riva and Toscano, German Patent No. 2,508,136, and Riva et al., U.S. Pat. No. 4,226,862); hydrocortamate (N,N-diethylglycine (11β)-11,17-dihydroxy-3,20-dioxopregn-4-en-21-yl ester, Pinson and Laubach, German Patent No. 1,016,708, and Richter and Schenck, German Patent No. 1,037,451), or its hydrochloride form (C₂₇H₄₁NO₆.HCl); hydrocortisone ((11β)-11,17,21-trihydroxypregn-4-ene-3,20-dione; Murray and Peterson, U.S. Pat. No. 2,602,769), its 21-acetate form (C₂₃H₃₂O₆), its 17-butyrate form (C₂₅H₃₆O₆), its 21-phosphate disodium salt form (C₂₁H₂₉Na₂O₈P), its 21-sodium succinate form (C₂₅H₃₃NaO₈), its 17-valerate form (C₂₆H₃₈O₆), or its cypionate form (Munson and Wilson, J Pharm Sci (1981) 70(2), 177-81), examples of brand name for hydrocortisone include Cortef Hydrocortone, examples of brand name for hydrocortisone cypionate include Cortef Oral Suspension; loteprednol etabonate ((11β,17α)-17[(ethoxycarbonyl)oxy]-11-hydroxy-3-oxoandrosta-1,4-diene-17-carboxylic acid chloromethyl ester; Bodor, Belgium Patent No. 889,563, and U.S. Pat. No. 4,996,335); mazipredone ((11β)-11,17-dihydroxy-21-(4-methyl-1-piperazinyl)pregna-1,4-diene-3,20-dione; Tuba et al., Hungarian Patent No. 150,350), or its hydrochloride form (C₂₆H₃₈N₂O₄.HCl); medrysone ((6α,11β)-11-hydroxy-6-methylpregn-4-ene-3,20-dione; Sebek et al., U.S. Pat. No. 2,864,837, and Spero and Thompson, U.S. Pat. No. 2,968,655); meprednisone ((16β)-17,21-dihydroxy-16-methylpregna-1,4-diene-3,11,20-trione; British Patent No. 901,092, and Rausser and Oliveto, U.S. Pat. No. 3,164,618), or its 21-acetate form (C₂₄H₃₀O₆); methylprednisolone ((6α,11β)-11,17,21-trihydroxy-6-methylpregna-1,4-diene-3,20-dione; Sebek and Spero, U.S. Pat. No. 2,897,218, and Gould, U.S. Pat. No. 3,053,832), its 21-acetate form (C₂₄H₃₂O₆), its 21-phosphate disodium salt form (C₂₂H₂₉Na₂O₈P), its 21-succinate sodium salt form (C₂₆H₃₃NaO₈), or its aceponate form (C₂₇H₃₆O₇), examples of brand name for methylprednisolone include Medrol-Oral; mometasone furoate ((11β,16α)-9,21-dichloro-17-[(2-furanylcarbonyl)oxy]-11-hydroxy-16-methylpregna-1,4-diene-3,20-dione; Shapiro, European Patent Application No. 57,401, and U.S. Pat. No. 4,472,393); paramethasone ((6α,11β,16α)-6-fluoro-11,17,21-trihydroxy-16-methylpregna-1,4-diene-3,20-dione; Edwards et al., J. Am. Chem. Soc. (1960) 82, 2318), its 21-acetate form (C₂₄H₃₁FO₆), its disodium phosphate form, or a mixture of its 21-acetate and disodium phosphate form; prednicarbate ((11β)-17[(ethoxycarbonyl)oxy]-11-hydroxy-21-(1-oxopropoxy)pregna-1,4-diene-3,20-dione; Stache et al., Germany Patent No. 2,735,110, and U.S. Pat. No. 4,242,334); prednisolone ((11β)-11,17,21-trihydroxypregna-1,4-diene-3,20-dione; Nobile, U.S. Pat. Nos. 2,837,464 and 3,134,718), its 21-acetate form (C₂₃H₃₀O₆), its 21-tert-butylacetate form (C₂₇H₃₈O₆; Sarrett, U.S. Pat. No. 2,736,734), its 21-hydrogen succinate form (C₂₅H₃₂O₈), its 21-succinate sodium salt form (C₂₅H₃₁NaO₈; Shull and Kita, German Patent No. 1,045,400), its 21-stearoylgylcolate form (C₄₁H₆₄O₈; Giraldi and Nannini, U.S. Pat. No. 3,171,846), its 21-m-sulfobenzoate sodium salt form (C₂₈H₃₁NaO₉S; (11β)-11,17-dihydroxy-21-[(3-sulfobenzoyl)oxy]pregna-1,4-diene-3,20-dione monosodium salt; Allais and Girault, U.S. Pat. No. 3,032,568, Joly and Warnant, U.S. Pat. No. 3,037,034), or its 21-trimethylacetate form (C₂₆H₃₆O₆; Joly and Warnant, U.S. Pat. No. 3,037,034), examples of brand name for prednisolone include Prelone, Delta-Cortef, Pediapred, Adnisolone, Cortalone, Deltacortril, Deltasolone, Deltastab, Di-Adreson F, Encortolone, Hydrocortancyl, Medisolone, Meticortelone, Opredsone, Panaafcortelone, Precortisyl, Prenisolona, Scherisolona, Scherisolone; prednisolone 21-diethylaminoacetate(N,N-diethylglycine (11β)-11,17-dihydroxy-3,20-dioxopregna-1,4-dien-21-yl ester; British Patent No. 862,370), or its hydrochloride form (C₂₇H₃₉NO₆.HCl); prednisolone sodium phosphate (11,17-dihydroxy-21-(phosphonooxy)pregna-1,4-diene-3,20-dione disodium salt; Sarett U.S. Pat. No. 2,789,117, and Elks and Phillipps, U.S. Pat. No. 2,936,313); prednisone (17,21-dihydroxypregna-1,4-diene-3,11,20-trione; Oliveto and Gould, U.S. Pat. No. 2,897,216, and Nobile, U.S. Pat. Nos. 2,837,464 and 3,134,718), or its 21-acetate form (C₂₃H₂₈O₆), examples of brand name for prednisone include Deltasone, Liquid Pred, Meticorten, Orasone 1, Orasone 5, Orasone 10, Orasone 20, Orasone 50, Prednicen-M, Prednisone Intensol, Sterapred, Sterapred DS, Adasone, Cartancyl, Colisone, Cordrol, Cortan, Dacortin, Decorti, Decortisyl, Delcortin, Dellacort, Delta-Dome, Deltacortene, Deltisona, Diadreson, Econosone, Encorton, Fernisone, Nisona, Novoprednisone, Panafcort, Panasol, Paracort, Parmenison, Pehacort, Predeltin, Prednicort, Prednicot, Prednidib, Predniment, Rectodelt, Ultracorten, Winpred; prednival ((11β)-11,21-dihydroxy-17-[(1-oxopentyl)oxy]pregna-1,4-diene-3,20-dione; Ercoli and Gardi, U.S. Pat. No. 3,152,154), or its 21-acetate form (C₂₈H₃₈O₇); prednylidene ((11β)-11,17,21-trihydroxy-16-methylenepregna-1,4-diene-3,20-dione; Mannhardt et al., Tetrahedron Letters (1960) 16, 21), or its 21-diethylaminoacetate hydrochloride form (C₂₈H₃₉NO₆.HCl; German Patent No. 1,134,074); rimexolone ((11β,16α,17β)-11-hydroxy-16,17-dimethyl-17-(1-oxopropyl)androsta-1,4-dien-3-one; Dutch Patent Application No. 7,300,313, and Woods et al., U.S. Pat. No. 3,947,478); rofleponide ((22R)-6α,9α-Difluoro-11β,21-dihydroxy-16α,17α-propylmethylenedioxypregn-4-ene-3,20-dione; Thalen and Wickstrom, Steroids (2000) 65(1), 16-23); tipredane ((11β, 17α)-17-(ethylthio)-9α-fluoro-11β-hydroxy-17-(methylthio) androsta-1,4-dien-3-one; Wojnar et al., Arzneimittelforschung (1986) 36(12), 1782-7); tixocortol ((11b)-11,17-dihydroxy-21-mercaptopregna-4-ene-3,20-dione; Simons et al., J Steroid Biochem (1980) 13, 311), or its 21-pivalate form (C₂₆H₃₈O₅S; (11β)-21-[(2,2-dimethyl-1-oxopropyl)thio]-11,17-dihydroxypregna-4-ene-3,20-dione; Torossian et al., German Patent No. 2,357,778, and U.S. Pat. No. 4,014,909); triamcinolone ((11β,16α)-9-fluoro-11,16,17,21-tetrahydroxypregna-1,4-diene-3,20-dione; Bernstein et al., U.S. Pat. No. 2,789,118, and Allen et al., U.S. Pat. No. 3,021,347), or its 16,21-diacetate form (C₂₅H₃₁FO₈; (11β,16α)-16,21-bis(acetyloxy)-9-fluoro-11,17-dihydroxypregna-1,4-diene-3,20)dione), examples of brand name for triamcinolone include Kenacort, Aristocort, Atolone, Sholog A, Tramacort-D, Tri-Med, Triamcot, Tristo-Plex, Trylone D, U-Tri-Lone; Triamcinolone acetonide ((11β,16α)-9-fluoro-11,21-dihydroxy-16,17-[1-methylethylidenebis(oxy)]pregna-1,4-diene-3,20-dione; Bernstein and Allen, U.S. Pat. No. 2,990,401, and Hydorn, U.S. Pat. No. 3,035,050), its 21-acetate crystal form, its 21-disodium phosphate form (C₂₄H₃₀FNa₂O₉P), or its 21-hemisuccinate form (C₂₈H₃₅FO₉); triamcinolone benetonide ((11β,16α)-21-[3-(benzoylamino)-2-methyl-1-oxopropoxy]-9-fluoro-11-hydroxy-16,17-[(1-methylethylidene)bis(oxy)]pregna-1,4-diene-3,20-dione; Cavazza et al., German Patent No. 2,047,218, and U.S. Pat. No. 3,749,712); and triamcinolone hexacetonide ((11β,16α)-21-3,3-dimethyl-1-oxobutoxy)-9-fluoro-11-hydroxy-16,17-[(1-methylethylidene)bis(oxy)]pregna-1,4-diene-3,20-dione; Nash and Naeger, U.S. Pat. No. 3,457,348). Preferably, the steroids comprises budesonide, testosterone, progesterone, estrogen, flunisolide, triamcinolone, beclomethasone, betamethasone, dexamethasone, fluticasone, methylprednisolone, prednisone, hydrocortisone, and mometasone. Another group of preferred steroids are mineralocorticoid steroids including aldosterone, deoxycorticosterone, deoxycorticosterone acetate and fludrocortisone. However, others are also suitable.

Also provided is a method for reducing or depleting adenosine levels, or treating hypersensitivity to adenosine, particularly in the lung, liver, heart and/or brain, or increasing levels of lung surfactant or of ubiquinone in the lung, heart or other tissues, and for treating various respiratory, lung and other diseases and their symptoms, by administering to a subject in need of such treatment a first active agent comprising the anti-sense oligo of the invention, and a second active agent comprising the AIS of chemical formula (Ia) and (Ib) exemplified by corticosteroids and dehydroepiandrosterones, analogues thereof, and pharmaceutically or veterinarily acceptable salts thereof, such as dehydroepiandrosterone sulfite (DHEA-S), and salts of the corticosteroids, and/or a ubiquinone of chemical formula (II) as described above, the active agents being present in amounts effective to reduce or deplete adenosine levels, or reduce adenosine hypersensitivity, or to increase lung surfactant levels or ubiquinone tissue levels, or to inhibit or control a variety of respiratory, lung and other diseases and conditions in the subject. Examples of non-glucocorticoid steroids that may be used to carry out this method are represented by the chemical formula (Ia) shown above.

Another group of preferred steroids for use in this invention are described below. The hydrogen atom at position 5 of the compound of chemical formula (Ia) may be present in the alpha or beta configuration, and the compound may comprise a mixture of both configurations. Compounds illustrative of compounds of chemical formula (I) above include DHEA, wherein R and R₁ each comprise hydrogen and the double bond is present; 16-alpha bromodehydroepiandrosterone, where R comprises Br, R1 comprises H, and the double bond is present; 16-alpha-fluorodehydroepiandrosterone, wherein R comprises F, R1 comprises H and the double bond is present; etiocholanolone, where R and R1 each comprise hydrogen and the double bond is absent (the single bond is present); and dehydroepiandrosterone sulphate (DHEA-S), wherein R comprises H, R1 comprises SO₂OM and M comprises sulphatide as defined above, and the double bond is present, among others. In the compound of formula I, R preferably comprises halogen, e.g. bromo, chloro, or fluoro, R1 comprises hydrogen, and the double bond is present. Most preferably the compound of Formula I comprises dehydroepiandrosterone sulphate and 16-α-fluorodehydroepiandrosterone. The compounds of formula I may be made in accordance with procedures known in the art, or employing variations thereof that will be apparent to those skilled in the art. See, for example, U.S. Pat. No. 4,956,355, UK Patent No. 2,240,472, EPO Patent Application No. 429,187, Patent Publication WO9104030A1; Abou-Gharbia M. et al., J. Pharm. Sci. 70: 1154-1157 (1981), Merck Index Monograph No. 7710, 11th Ed. (1989). Other preferred non-glucocorticoid steroids are those of the formulas (III) and (IV), wherein R15 and R16 together are ═O, or where R5 is OH, or where R5 is —OSO2R20, or where R20 is H. Others, however, are also preferred and are encompassed by this patent

“Corticosteroid”, as used herein, means 21-carbon steroid hormone corticoids that bind to glucocorticoid receptors, having the chemical formula of (Ib). Corticosteroids are agonists for the glucocorticoid steroid receptor(s) and interact to promote a transcriptional response. The corticosteroids and other AIS may be used in conjunction with, and for reducing the amount of the oligo(s) employed for reducing inflammation and lung allergy(ies), reducing or depleting levels of; or reducing sensitivity to, adenosine, reducing adenosine receptor levels, producing bronchodilation, and/or for increasing levels of ubiquinone or lung surfactant in a subject, or for treating bronchoconstriction, lung inflammation or allergies or a respiratory or lung disease or condition. The anti-inflammatory steroid(s) may be administered per se or in the form of pharmaceutically acceptable salt, as discussed above. In general, the anti-inflammatory steroid(s), and its(their) salt(s) and crystal forms are suitable, and may be administered in a dosage of about 0.01, about 0.1, about 0.4, about 1, about 5, about 10, about 20 to about 4, about 30, about 70, about 100, about 300, about 1,000, about 3600 mg/kg body weight. These active compounds may be administered once or several times a day, or in any other regime, upon adjustment of the dose in accordance with the dosages of the other agents being administered.

The term “ubiquinone”, as used herein, refers to a family of compounds having structures based on a ω3-dimethoxy-5-methyl benzoquinone nucleus with a variable terpenoid acid chain containing on to twelve non-unsaturated trans-isoprenoid units. Such compounds are also known in the art as “Coenzyme Qₙ”, wherein n comprises 1 to 12, preferably a comprising 1 to 10, and may be referred to herein as compounds represented by the following chemical formula 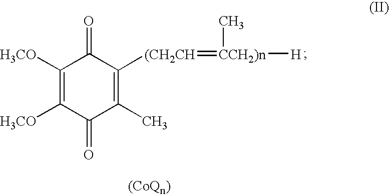
wherein n comprises 1 to 10. In the method of the invention, another preferred ubiquinone is a compound according to the above formula, where n comprises 6 to 10, i.e. Coenzyme Q₆₋₁₀, and most preferably wherein n comprises 10, i.e. Coenzyme Q₁₀.

As discussed above, the “active agents or compounds” may be administered per se or in the form of pharmaceutically acceptable salts, or in the same formulation with the other active agents of the invention, e.g. corticosteroid(s) and/or ubiquinone(s) and the anti-sense oligo, either systemically or topically. In general, they are administered in an amount effective to treat respiratory conditions including bronchoconstriction, respiratory inflammation and allergies, allergic rhinitis, pulmonary hypertension and fibrosis, apnea, sepsis, emphysema, cancers, asthma, COPD, RDS, CF, ARDS, and the like, and/or to off-set lung surfactant depletion or ubiquinone depletion in the lungs and/or heart of the subject if induced by the administration of the anti-inflammatory steroid of the invention. The ubiquinone is preferably administered in a total amount per day of of about 0.1, about 1, about 5, about 10, about 15, about 30 to about 50, about 100, about 150, about 300, about 600, about 900, about 1200 mg/kg body weight per day. More preferred are about 1 to about 150 mg/kg, about 30 to about 100 mg/kg, and most preferred about 5 to about 50 mg/kg. The ubiquinone may be administered in one dose (once or several times a day), and its dose may be adjusted as is known in the art, depending on whether it is administered alone, or with the oligo and/or the anti-inflammatory steroid, and their amounts used. The dosage of the ubiquinone will vary depending upon the condition of the subject and route of administration. The ubiquinone may be administered by itself, or as a mixture of ubiquinones of varying side chain lengths, or concurrently, jointly prior to or subsequent to the anti-sense oligo and/or the anti-inflammatory steroid, for treating the overall symptoms described here, and/or the various diseases associated with them, including asthma, COPD, allergic rhinitis, pulmonary hypertension, vasoconstriction and fibrosis, and others described above. The phrase “concurrently administering”, as used herein, means that the steroid, e.g. DHEA, DHEA-S or analogs of formulas (Ia) and (Ib), the anti-sense oligos, and the ubiquinone of chemical formula (II) are administered either (a) simultaneously in time, preferably by formulating the two active agents together in a common pharmaceutical carrier, or (b) at different times during the course of a common treatment schedule through the same or different routes of administration. In the latter case, for example the oligo may be administered once a week or its administration may be varied in accordance with its duration of action, while steroid(s) and ubiquinone(s) is(are) administered at times sufficiently close so that, in addition to its direct effect, the ubiquinone will be also off-setting any ubiquinone depletion in the subject's tissues, e.g. lungs and heart. This timing helps to prevent or counter-balance any deterioration of tissue, e.g. lung sand heart function that may result from the administration of the steroids or analogs thereof. Where the ubiquinone is formulated with a pharmaceutically acceptable carrier and other oral formulation components, it may be administered separately from the steroid and/or the oligo. For example, the steroid and the oligo may be administered into the respiration, by inhalation, nasally or into the lungs (by instillation) of the subject whereas the ubiquinone may be administered systemically. The ubiquinone may be formulated by any of the techniques set forth above.

The composition and formulations of this invention are highly efficacious for preventing and treating diseases and conditions associated with bronchoconstriction, difficult breathing, impeded and obstructed lung airways, allergy(ies), inflammation and surfactant depletion, among others. Examples of diseases and conditions which are suitably treated by the present method are diseases and conditions, including Acute Respiratory Distress Syndrome (ARDS), asthma, adenosine administration e.g. in the treatment of Supra Ventricular Tachycardia (SVT) and other arrhythmias, and in stress tests to hyper-sensitized individuals, ischemia, renal damage or failure induced by certain drugs, infantile respiratory distress syndrome, pain, cystic fibrosis, pulmonary hypertension, pulmonary vasoconstriction, emphysema, chronic obstructive pulmonary disease (COPD), lung transplantation rejection, pulmonary infections, and cancers such as leukemias, lymphomas, carcinomas, and the like, including colon cancer, breast cancer, lung cancer, pancreatic cancer, hepatocellular carcinoma, kidney cancer, melanoma, hepatic metastases, etc., as well as all types of cancers which may metastasize or have metastasized to the lung(s), including breast and prostate cancer. The invention will be mostly described with respect to the adenosine receptors as targets, although data on other targets is also provided, but is similarly applicable to any other target including the listed targets, with respect to the administration of anti-sense oligos. The examples provided below show a complete inhibition of adenosine receptor associated symptoms in a rabbit model for human bronchoconstriction, allergy(ies) and inflammation as well as the elimination of the ability of the adenosine receptor agonist par excellence, adenosine, to cause bronchoconstriction in hyper-responsive monkeys, which are animal models for human hyper-responsiveness to adenosine receptor agonists. The pharmaceutical composition and formulations of the invention, therefore, are suitable for preventing and alleviating the symptoms associated with stimulation of adenosine receptors, such as the adenosine A₁, A₂ₐ, A_{2b}, and A₃ receptors, as well as other single or multiple targets. The compositions and formulations of this invention, thus, are also suitable for prevent the untoward side effects of adenosine-mediated hyperresponsiveness in certain individuals, which are generally seen in diseases affecting respiratory activity.

The method of the present invention may be used to treat airway and lung diseases and conditions in a subject of any kind and for any reason, for example, to reduced or eliminated with the intention that the adenosine content of anti-sense compounds, so as to prevent liberation of adenosine upon anti-sense degradation. Examples of diseases and conditions, which may be treated preventatively, prophylactically and therapeutically with the compositions and formulations of this invention, are pulmonary vasoconstriction, inflammation, allergies, asthma, allergic rhinitis, impeded respiration, Acute Respiratory Distress Syndrome (ARDS), renal damage and failure associated with ischemia as well as the administration of certain drugs, side effects associated with adenosine administration e.g. in SupraVentricular Tachycardia (SVI) and in adenosine stress tests, infantile Respiratory Distress Syndrome (infantile RDS), ARDS, pain, cystic fibrosis, pulmonary hypertension, pulmonary vasoconstriction, emphysema, chronic obstructive pulmonary disease (COPD), lung transplantation rejection, pulmonary infections, and cancers such as leukemias, lymphomas, carcinomas, and the like, e.g. colon cancer, breast cancer, lung cancer, pancreatic cancer, hepatocellular carcinoma, kidney cancer, melanoma, metastatic cancer such as hepatic metastases, lung, breast and prostate metastases, among others. The present compositions and formulations are suitable for administration before, during and after other treatments, including radiation, chemotherapy, antibody therapy, phototherapy and cancer, and other types of surgery. The present compositions and formulations may also be administered effectively as a substitute for therapies that have significant negative side effects. The terms “anti-sense” oligonucleotides generally refers to small, synthetic oligonucleotides, resembling single- and double-stranded DNA and RNA, which in this patent are applied to the inhibition of gene expression, e.g. by inhibition of a gene or target messenger RNA (mRNA). See, e.g. Milligan, J. F. et al., J. Med. Chem. 36(14), 1923-1937 (1993); Sharp, P.A. Genes & Development 15, 485-490, 2001; the relevant portion of which is hereby incorporated in its entirety by reference. For consistency's sake, all RNAs, DNAs and oligonucleotides are represented in this patent by a single strand in the 5′ to 3′ or 3′ to 5′ direction, when read from left to right although their complementary and double-stranded sequence(s) is (are) also encompassed within the four corners of the invention. In addition, all nucleotide bases and amino acids are represented utilizing the recommendations of the IUPAC-IUB Biochemical Nomenclature Commission, or by the known 3-letter code (for amino acids). Nucleotide sequences are presented herein by single strand only, in the 5′ to 3′ direction, from left to right. In addition, nucleotide and amino acids are represented herein in the manner recommended by the IUPAC-IUB Biochemical Nomenclature Commission, or (for amino acids) by three letter code, in accordance with 37 CFR '1.822 and established usage. See, e.g., PatentIn User Manual, 99-102 (November 1990) (U.S. Patent and Trademark Office, Office of the Assistant Commissioner for Patents, Washington, D.C: 20231); U.S. Pat. No. 4,871,670 to Hudson et al. at col. 3, lines 20-43. The present method utilizes anti-sense agents to inhibit or down-regulate gene expression of target genes, including those listed in Tables 1 and 2 below. This is generally attained by hybridization of the anti-sense oligonucleotides to coding (sense) sequences of a targeted messenger RNA (mRNA), as is known in the art. The oligos of this invention may be obtained by first selecting fragments of a target nucleic acid having at least 4 contiguous nucleic acids selected from the group consisting of G and C, and then obtaining a first oligonucleotide 4 to 70 nucleotides long which comprises the selected fragment and preferably has a C and G nucleic acid content of up to and including about 20%, about 15%. The oligonucleotide(s) (oligo(s)) may include at least one unmethylated cytosine-guanine (CpG) dinucleotide. The CpG dinucleotide may be substituted for a methylated cytosine present in the anti-sense oligonucleotide(s). The CpG dinucleotide is n immunostimulating sequence and affects the immune response in a subject by activating natural killer cells (NK) or redirecting a subject's immune response from a Th2 to a Th1 response by inducing monocytic and other cells to produce Th1 cytokines. The oligo(s) containing at least one unmethylated CpG can be used for treating and/or preventing respiratory and pulmonary diseases including bronchoconstriction, impaired airways, decreased lung surfactant, asthma, rhinitis, acute respiratory distress syndrome (ARDS), infantile or maternal RDS, chronic obstructive pulmonary disease (COPD), allergies, impeded respiration, lung pain, cystic fibrosis (CF), infectious diseases, cancers such as leukemias, lung and colon cancer, and the like, and diseases whose secondary effects afflict the lungs. A “CpG” or “CpG motif” refers to nucleotides having a cytosine followed by a guanine linked by a phosphate bond. The term “methylated CpG” refers to the methylation of the cytosine on the pyrimidine ring, usually occurring the 5-position of the pyrimidine ring. The term “unmethylated CpG” refers to the absence of methylation of the cytosine on the pyrimidine ring. Methylation, partial removal, or removal of an unmethylated CpG motif in an oligo(s) is believed to reduce its effect Methylation or removal of all unmethylated CpG motifs in an oligo(s) substantially reduces its effect. The effect of methylation or removal of a CpG motif is “substantial” if the effect is similar to that of an oligonucleotide that does not contain a CpG motif. Preferably the CpG oligonucleotide is in the range of about 8 to 30 bases in size. The oligo(s) can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet Let. 27:4051-4054, 1986; Froehler et al., Nucl. Acid. Res. 14:5399-5407, 1986; Garegg et al., Tet Let. 27:4055-4058, 1986, Gaffney et al., Tet. Let 29:2619-2622, 1988). These chemistries can be performed by a variety of automated oligonucleotide synthesizers available in the market Alternatively, CpG dinucleotides can be produced on a large scale in plasmids, (see Sambrook, T., et al., Molecular Cloning: A Laboratory Manual, Cold Spring Harbor laboratory Press, New York, 1989) which after being administered to a subject are degraded into an oligo(s). An oligo(s) can be prepared from existing nucleic acid sequences (e.g., genomic or cDNA) using known techniques, such as those employing restriction enzymes, exonucleases or endonucleases. The exogenously administered agents of the invention decrease the levels of mRNA and protein encoded by the target gene and/or cause changes in the growth characteristics or shapes of the thus treated cells. See, Milligan et al. (1993); Helene, C. and Toulme, J. Biochim. Biophys. Acta 1049, 99-125 (1990); Cohen, J. S. D., Ed., Oligodeoxynucleotides as Anti-sense Inhibitors of Gene Expression; CRC Press: Boca Raton, Fla. (1987), the relevant portion of which is hereby incorporated in its entirety by reference.

The treatment of this invention enhances the effects of the oligonucleotide and the anti-inflammatory steroid(s) and/or ubiquinone(s) by combining them, either simultaneously, sequentially or separately, for reducing or depleting levels of, or reducing sensitivity to, adenosine, reducing levels of receptor(s), producing bronchodilation, or for increasing levels of ubiquinone or lung surfactant in a subject's tissue (s), or treating bronchoconstriction, lung inflammation or allergies or a respiratory or lung disease or condition, and/or for alleviating bronchoconstriction or lung inflammation or allergy(ies), or ubiquinone or lung surfactant depletion or hyposecretion, in a subject. When administered in combination, the dose of the oligonucleotide or the steroid(s) orubiquinone(s) may be decreased since they potentiate each other's effect. These agents may be administered before, simultaneously with, and/or after each other's administration. Accordingly, the details of administration of the effect enhancer including its amount, route, formulation, method, target organ and/or tissue may be determined as described throughout this specification. Similarly, other therapeutic or bioactive agents may be employed in accordance with this invention. Kits comprising the various agents described above are also part of this invention.

As used herein, “anti-sense oligonucleotide or anti-sense oligo” is generally a short sequence of synthetic nucleotide that hybridizes to any segment of a mRNA encoding a targeted protein under appropriate hybridization conditions and which, upon hybridization, causes a decrease in gene expression of the targeted protein. The terms “desAdenosine” (desA),“des-thymidine” (desT) and “des-uridine (desU) refer to oligonucleotides substantially lacking either adenosine (desA) or thymidine (desT) (uracil (desU)). In some instances, the desA or desT (desU) sequences are naturally occurring, and in others they may result from substitution of an undesirable nucleotide (A) by another lacking its undesirable activity, such as acting as an agonist or having a triggering effect at the adenosine A receptor(s). In the present context, the substitution is generally accomplished by substitution of A with a “universal or alternative base”, presently known in the art or to be ascertained at a later time. As used herein, the terms “prevent”, “preventing”, “treat” or “treating” refer to a preventative, prophylactic, maintenance, or therapeutic treatment which decreases the likelihood that the subject administered such treatment will manifest symptoms associated with adenosine receptor stimulation. The term “down-regulate” refers to inducing a decrease in production, secretion or availability and, thus, a decrease in concentration, of intracellular target product, be it a receptor, e.g. adenosine A₁, A_{2b}, A₃, bradyknin 2B, GATA-3, or other receptors, or produce a stimulatory effect on a receptor such as the adenosine A₂ₐ receptor. The present technology relies on the design of anti-sense oligos targeted to genea and mRNAs associated with ailments involving nasal and lung airway(s) (respiratory tract) pathology(ies), and on their modification to reduce the potential occurrence of undesirable side effects caused by their release of adenosine upon breakdown, while preserving their activity and efficacy for their intended purpose. In this manner, the inventor targets a specific gene to design one or more anti-sense single or double stranded DNA or RNA oligonucleotide(s) (oligos) that selectively bind(s) to the corresponding gene or mRNA, and then reduces, if necessary, their content of adenosine via substitution with an alternative or a universal base, or an adenosine analog incapable of significantly, or having substantially reduced ability for, activating or antagonizing adenosine A₁, A_{2b} or A₃ receptors or which may act as an agonist at the adenosine A₂ₐ receptor. Any number of adenosines present may be substituted by an alternative and/or universal base, such as heteroaromatic bases, which binds to a thymidine or uridine base but has less than about 0.3 of the adenosine base agonist or antagonist activity at the adenosine A₁, A₂ₐ, A_{2b} and A₃ receptors. Based on his prior experience in the field, the inventor reasoned that in addition to “downregulating” specific genes, he could increase the effect of the agent(s) administered by either selecting segments of RNA that are devoid, or have a low content, of thymidine (T) or uridine (U), or alternatively, substitute one or more adenosine(s) present in the designed oligonucleotide(s) with other nucleotide bases, so called universal bases, which bind to thymidine but lack the ability to activate adenosine receptors and otherwise exercise the constricting effect of adenosine in the lungs, etc. Given that adenosine (A) is a nucleotide base complementary to thymidine (T) or uridine (U), wherein when a U appears in the RNA, the anti-sense oligo will have an A at the same position.

In one aspect of this invention, the anti-sense oligonucleotide has a sequence which specifically binds to a portion or segment of a mRNA molecule which encodes or regulates the production of a protein associated with impeded breathing, allergy(ies), lung inflammation, depletion of lung surfactant or lowering of lung surfactant, airway obstruction, bronchitis, and the like. One effect of this binding is to reduce or even prevent the translation of the corresponding mRNA and, thereby, reduce the available amount of target protein in the subject's lung. In one preferred embodiment of this invention, the phosphodiester residues of the anti-sense oligonucleotide are modified or substituted. Chemical analogs of oligonucleotides with modified or substituted phosphodiester residues, e.g., to the methylphosphonate, the phosphotriester, the phosphorothioate, the phosphorodithioate, or the phosphoramidate, 2′ methoxy ethyl and similar modifications; which increase the in vivo stability of the oligonucleotide are particularly preferred. The naturally occurring phosphodiester linkages of oligonucleotides are susceptible to some degree of degradation by cellular nucleases. Many of the residues proposed herein, on the contrary, are highly resistant to nuclease degradation. See, Milligan et al.; Cohen, J. S. D., supra. In another preferred embodiment of the invention, the oligonucleotides may be protected from degradation by adding a “3′-end cap” by which nuclease-resistant linkages are substituted for phosphodiester linkages at the 3′ end of the oligonucleotide. See, Tidd, D. M. and Warenius, H.M., Be. J. Cancer 60: 343-350 (1989); Shaw, J. P. et al., Nucleic Acids Res. 19: 747-750 (1991), the relevant section of which are incorporated in their entireties herein by reference. Phosphoramidates, phosphorothioates, and methylphosphonate linkages all function adequately in this manner for the purposes of this invention, as do 2′ modifications, such as 2′ methoxy ethyl, and the like. The more extensive the modification of the phosphodiester backbone the more stable the resulting agent, and in many instances the higher their RNA affinity and cellular permeation. See, Milligan, et al., supra. In addition, a plurality of substitutions to the carbohydrate ring are also known to improve stability of nucleic acids. Thus, the number of residues which may be modified or substituted will vary depending on the need, target, and route of administration, and may be from 1 to all the residues, to any number in between. Many different methods for replacing the entire phosphodiester backbone with novel linkages are known. See, Millikan et al, supra. Preferred backbone analogue residues include phosphoramidate, phosphorothioate, methylphosphonate, phosphorotriester, phosphotriester, thioformacetal, phosphorodithioate, phosphoramidate, formacetal, triformacetal, thioether, carbamate, boranophosphate, 3′-thioformacetal, 5′-thioether, carbonate, C₅-substituted nucleotides, 5′-N-carbamate, sulfate, sulfonate, sulfamate, sulfonamide, sulfone, sulfite, 2′-O methyl, sulfoxide, sulfide, hydroxylamine, methylene(methylimino) (, methoxymethyl (MOM), and methoxyethyl (MOE), and methyleneoxy(methylimino) (MOMI) residues, and combinations thereof. Phosphorothioate and methylphosphonate-modified oligonucleotides are particularly preferred due to their availability through automated oligonucleotide synthesis. See, Millikan et al, supra. Where appropriate, the agent of this invention may be administered in the form of their pharmaceutically acceptable salts, or as a mixture of the anti-sense oligonucleotide and its salt. In another embodiment of this invention, a mixture of different anti-sense oligonucleotides or their pharmaceutically acceptable salts is administered. A single agent of this invention has the capacity to attenuate the expression of a target mRNA and/or various agents to enhance or attenuate the activity of a pathway. By means of example, the present method may be practiced by identifying all possible deoxyribonucleotide segments which are low in thymidine (T), ribonucleotides that are low in uridine (U), or deoxynucleotide segments low in adenosine (A) of about 7 or more mononucleotides, preferably up to about 60 mononucleotides, more preferably about 10 to about 36 mononucleotides, and still more preferably about 12 to about 21 mononucleotides, in a target mRNA or a gene, respectively. This may be attained by searching for nucleotide segments within a target sequence which are low in, or lack thymidine (DNA) or uridine (RNA), a nucleotide which is complementary to adenosine, or that are low in adenosine (gene), that are 7 or more nucleotides long. In most cases, this search typically results in about 10 to 30 such sequences, i.e. naturally lacking or having less than about 40% adenosine, anti-sense oligonucleotides of varying lengths for a typical target mRNA of average length, i.e., about 1800 nucleotides long. Those with high content of T, U or A, respectively, may be fixed by substitution of a universal base for one or more As. The agent(s) of this invention may be of any suitable length, including but not limited to, about 7 to about 60 nucleotides long, preferably about 12 to about 45, more preferably up to about 30 nucleotides long, and still more preferably up to about 21, although they may be of other lengths as well, depending on the particular target and the mode of delivery. The agent(s) of the invention may be directed to any and all segments of a target RNA. One preferred group of agent(s) includes those directed to an mRNA region containing a junction between an intron and an exon. Where the agent is directed to an intron/exon junction, it may either entirely overlie the junction or it may be sufficiently close to the junction to inhibit the splicing-out of the intervening exon during processing of precursor mRNA to mature mRNA, e.g. with the 3′ or 5′ terminus of the anti-sense oligonucleotide being positioned within about, for example, within about 2 to 10, preferably about 3 to 5, nucleotide of the intron/exon junction. Also preferred are anti-sense oligonucleotides which overlap the initiation codon, and those near the 5′ and 3′ termini of the coding region. The flanking regions of the exons may also be targeted as well as the spliced segments in the precursor mRNAs. The mRNA sequences of the adenosine receptors and of many other targets are derived from the DNA base sequence of the gene expressing either receptors, e.g. the adenosine receptors, the enzymes, factors, or other targets associated with airway disease. For example, the sequence of the genomic human A₁ adenosine receptor is known and is disclosed in U.S. Pat. No. 5,320,963 to Stiles, G., et al. The A₃ adenosine receptor has been cloned, sequenced and expressed in rat (see, Zhou, F., et al., P.N.A.S. (USA) 89: 7432 (1992)) and human (see, Jacobson, M. A., et al., U.K. Patent Application No. 9304582.1 (1993)). The sequence of the adenosine A_{2b} receptor gene is also known. See, Salvatore, C. A., Luneau, C. J., Johnson, R. G. and Jacobson, M., Genomics (1995), the relevant portion of which is hereby incorporated in its entirety by reference. The sequences of many of the remaining exemplary target genes are also known. See, GenBank, NIH. The sequences of those genes whose sequences are not yet available may be obtained by isolating the target segments applying technology known in the art. Once the sequence of the gene, its RNA and/or the protein are known, an anti-sense oligonucleotides may be produced according to this invention as described above to reduce the production of the targeted protein in accordance with standard techniques. The sequences for the adenosine A₂ₐ bradykinin, and other genes as well as methods for preparation of oligonucleotides are also known as those of many other target genes and mRNAs for which this invention is suitable. Thus, anti-sense oligonucleotides that downregulate the production of target sequences associated with airway disease, including the adenosine A₁, A₂ₐ, A_{2b}, A₃, bradykinin, GATA-3, COX-2, and many other receptors, may be produced in accordance with standard techniques. Examples of diseases and conditions which are suitably treated by the present method are diseases and conditions, including Acute Respiratory Distress Syndrome (ARDS), asthma, adenosine administration e.g. in the treatment of SupraVentricular Tachycardia (SVT) and other arrhythmias, and in stress tests to hyper-sensitized individuals, ischemia, renal damage or failure induced by certain drugs, infantile respiratory distress syndrome, pain, cystic fibrosis, pulmonary hypertension, pulmonary vasoconstriction, emphysema, chronic obstructive pulmonary disease (COPD), pulmonary transplantation rejection, pulmonary infections, and cancers such as leukemias, lymphomas, carcinomas, and the like, including colon cancer, breast cancer, lung cancer, pancreatic cancer, hepatocellular carcinoma, kidney cancer, melanoma, hepatic metastases, etc., as well as all types of cancers which may metastasize or have metastasized to the lung(s), including breast and prostate cancer.

The adenosine receptors discussed above are mere examples of the high power of the inventor's technology. In fact, a large number of genes may be targeted in a similar manner by the present agent(s), to reduce or down-regulate protein expression. This targeting may be attained by selecting a single target, or multiple targets. In the latter case, the oligos targeted to different sequences may be mixed for their administration or they may be multiple targeted anti-sense oligos (MTAs) in accordance with one embodiment of this invention; that is, the MTA sequence binds to more than one target polynucleotide, be it DNA or RNA. By means of example, if the target disease or condition is one associated with impeded or reduced breathing, bronchoconstriction, chronic bronchitis, pulmonary bronchoconstriction and/or hypertension, chronic obstructive pulmonary disease (COPD), pulmonary transplantation rejection, pulmonary infections, allergy, asthma, cystic fibrosis, respiratory distress syndrome, cancers, which either directly or by metastasis afflict the lung, the present method may be applied to a list of potential target mRNAs, which includes the targets listed in Table 1 and Table 2 below, among others. The anti-sense agent(s) of the invention have a low A content to prevent its liberation upon in vivo degradation of the agent(s). For example, if the system is the pulmonary or respiratory system, a large number of genes is involved in different functions, including those listed in Table 1 below.

TABLE 1
Pulmonary and Inflammatory Targets

| | |
|---|---|
| NFκB Transcription Factor | Interleukin-8 Receptor (IL-8 R) |
| Interleukin-5 Receptor (IL-5R) | Interleukin-4 Receptor (IL-4R) |
| Interleukin-3 Receptor (IL-3R) | Interleukin-1β (IL-1β) |
| Interleukin-1β Receptor (IL-1βR) | Eotaxin |
| Tryptase | Major Basic Protein |
| β2-adrenergic Receptor Kinase | Endothelin Receptor A |
| Endothelin Receptor B | Preproendothelin |
| Bradykinin B2 Receptor (B2BR) | IgE (High Affinity Receptor) |
| Interleukin-1 (IL-1) | Interleukin 1 Receptor (IL-1 R) |
| Interleukin-9 (IL-9) | Interleukin-9 Receptor (IL-9 R) |
| Interleukin-11 (IL-11) | Interleukin-11 Receptor (IL-11 R) |
| Inducible Nitric Oxide Synthase | Cyclooxygenase (COX) |
| Intracellular Adhesion Molecule 1 (ICAM-1) | Vascular Cellular Adhesion Molecule |
| Substance P | (VCAM) |
| Rantes | Endothelial Leukocyte Adhesion Molecule |
| | Endothelin ETA Receptor (ELAM-1) |
| Cyclooxygenase-2 (COX-2) | GM-CSF, Endothelin-1 |
| Monocyte Activating Factor | Neutrophil Chemotactic Factor |
| Neutrophil Elastase | Defensin 1, 2, 3 |
| Muscarinic Acetylcholine Receptors | Platelet Activating Factor |
| Tumor Necrosis Factor α | 5-lipoxygenase |
| Phosphodiesterase IV | Substance P |
| Substance P Receptor | Histamine Receptor |
| Chymase | CCR-1 CC Chemokine Receptor |
| Interleukin-2 (IL-2) | Interleukin-4 (IL-4) |
| Interleukin-12 (IL-12) | Interleukin-5 (IL-5) |
| Interleukin-6 (IL-6) | Interleukin-7 (IL-7) |
| Interleukin-8 (IL-8) | Interleukin-12 Receptor (IL-12R) |
| Interleukin-7 Receptor (IL-7R) | Interleukin-1 (IL-1) |
| Interleukin-14 Receptor (IL-14R) | Interleukin-14 |
| CCR-2 CC Chemokine Receptor | CCR-3 CC Chemokine Receptor |
| CCR-4 CC Chemokine Receptor | CCR-5 CC Chemokine Receptor |
| Prostanoid Receptors | GATA-3 Transcription Factor |
| Neutrophil Adherence Receptor | MAP Kinase |
| Interleukin-15 (IL-15) | Interleukin-15 Receptor (IL-15R) |
| Interleukin-11 (IL-11) | Interleukin-11 Receptor (IL-11R) |
| NFAT Transcription Factors | STAT 4 |
| MIP-1α | MCP-2 |
| MCP-3 | MCP-4 |
| Cyclophillin (A, B, etc.) | Phospholipase A2 |
| Basic Fibroblast Growth Factor | Metalloproteinase |
| CSBP/p38 MAP Kinase | Tryptase Receptor |
| PDG2 | Interleukin-3 (IL-3) |
| Interleukin-10 (IL-10) | Cyclosporin A —Binding Protein |
| FK506-Binding Protein | α4β1 Selectin |
| Fibronectin | α4β7 Selectin |
| cMad CAM-1 | LFA-1 (CD11a/CD18) |
| PECAM-1 | LFA-1 Selectin |
| C3bi | PSGL-1 |
| E-Selectin | P-Selectin |
| CD-34 | L-Selectin |
| p150,95 | Mac-1 (CD11b/CD18) |
| Fucosyl transferase | VLA-4 |
| STAT-1 | STAT-2 |
| CD-18/CD11a | CD11b/CD18 |
| ICAM2 and ICAM3 | C5a |
| CCR3 (Eotaxin Receptor) | CCR1, CCR2, CCR4, CCR5 |
| LTB-4 | AP-1 Transcription Factor |
| Protein kinase C | Cysteinyl Leukotriene Receptor |
| Tachykinnen Receptors (tach R) | IκB Kinase 1 & 2 |
| Interleukin-2 Receptor (IL-2R) | (e.g., Substance P, NK-1 & NK-3 Receptors) |
| STAT 6 | c-mas |
| NF-Interleukin-6 (NF-IL-6) | Interleukin-10 Receptor (IL-10R) |
| Interleukin-3 (IL-3) | Interleukin-2 Receptor (IL-2R) |
| Interleukin-13 (IL-13) | Interleukin-12 Receptor (IL-12R) |
| Interleukin-14 (IL-14) | Interleukin-6 Receptor (IL-6R) |
| Interleukin-16 (IL-16) | Interleukin-13 Receptor (IL-13R) |
| Medullasin | Interleukin-16 Receptor (IL-16R) |
| Adenosine A₁ Receptor (A₁ R) | Tryptase-I |
| Adenosine A_{2b} Receptor (A_{2b} R) | Adenosine A₃ Receptor (A₃ R) |
| β Tryptase | STAT-3 |
| Adenosine A₂ₐ Receptor (A₂ₐ R) | IgE Receptor β Subunit (IgE R β) |
| Fc-epsilon receptor CD23 antigen | IgE Receptor α Subunit (IgE R α) |
| IgE Receptor Fc Epsilon Receptor (IgERFc ξ R) | Substance P Receptor |
| Histidine decarboxylase | Tryptase-1 |
| Prostaglandin D Synthase | Easinophil Cationic Protein |
| Eosinophil Derived Neurotoxin | Eosinophil Peroxidase |
| Endothelial Nitric Oxide Synthase | Endothelial Monocyte Activating Factor |
| Neutrophil Oxidase Factor | Cathepsin G |
| Macrophage Inflammatory Protein-1- | Interleukin-8 Receptor α Subunit (IL-8 Rα) |
| Alpha/Rantes Receptor | Endothelin Receptor ET-B |
| H2A histone family, member N | Tubulin, beta polypeptide |
| ELL gene (11-19 lysine-rich leukemia gene) | 7-dehydrocholesterol reductase |
| ADP-ribosylation factor-like 7 | Karyopherin alpha 2 (RAG cohort 1, importin alpha 1) |
| EST (AI038433) | EST (AI122689) |
| EST (AI092623) | ESTs (AI095492) |
| ESTs (AI138216) | ESTs (AI128305) |
| ESTs (AI125228) | ESTs (AI041482) |
| ESTs (AI051839) | Homo sapiens mRNA; cDNA DKFZp434A1716 |
| ESTs (AI096522) | ESTs (AI122807) |
| ESTs (AI041212) | EST (AI125651) |
| Enolase 1, (alpha) | EST (AI024215) |
| EST (AI034360) | Homo sapiens mRNA; cDNA DKFZp564H0764 |
| Homo sapiens mRNA for KIAA1363 protein, partial cds | |
| Potassium voltage-gated channel, shaker-related subfamily, beta member 2 | |
| ER-associated DNAJ; ER-associated Hsp40 co-chaperone; hDj9; ERj3 | |
| ESTs, Weakly similar to p38 protein [H. sapiens] (AA906703) | |
| CGI-142 | ESTs (AA463249) |
| Homo sapiens clone 25058 mRNA sequence | ESTs (R49144) |
| Squamous cell carcinoma antigen 1 | ESTs (AA425700) |
| Myosin X | ESTs (AA459692) |
| Epithelial protein lost in neoplasm beta | CD44 antigen (homing function and |
| | Indian blood group system) |
| Coagulation factor III (thromboplastin, tissue factor) | |
| ESTs (AA909635) | Adducin 1 (alpha) |
| 5′ Nucleotidase (CD73) | |
| ESTs, Moderately similar to semaphorin C [M. musculus] (AA293300) | |
| ESTs (AA278764) | ESTs (AA678160) |
| Calmodulin 2 (phosphorylase kinase, delta) | ESTs (R42770) |
| Chloride intracellular channel 1 | High-mobility group (nonhistone chromosomal) protein 17 |
| Ubiquitin carrier protein | Tubulin, alpha 1 (testis specific) |
| Transglutaminase 2 (C polypeptide, protein-glutamine-gamma-glutamyltransferase) | |
| Sparc/osteonectin, cwcv and kazal-like domains proteoglycan (testican) | |
| Proteasome (prosome, macropain) 26S subunit, non-ATPase, 2 | |
| Tubulin, beta polypeptide | Filamin B, beta (actin-binding protein-278) |
| Stanniocalcin | |
| Low density lipoprotein receptor (familial hypercholesterolemia) | |
| Plectin 1, intermediate filament binding protein, 500 kD | |
| S100 calcium-binding protein A2 | Immediate early response 3 |
| Calpain, large polypeptide L2 | Pleckstrin homology-like domain, family A, member 1 |
| Melanoma adhesion molecule | |
| CD44 antigen (homing function and Indian blood group system) | |
| Programmed cell death 5 | Hexokinase 1 |
| Vascular endothelial growth factor | Integrin, alpha 2 (CD49B, alpha 2 subunit of VLA-2 receptor) |
| Calumenin | Syntaxin 11 |
| Diphtheria toxin receptor (heparin-binding epidermal growth factor-like growth factor) | |
| Fn14 for type I transmenmbrane protein | Nef-associated factor 1 |
| High-mobility group (nonhistone chromosomal) protein isoforms I and Y | |
| Catechol-O-methyltransferase | C-terminal binding protein 1 |
| Collagen, type XVII, alpha 1 | ESTs (N58473) |
| Farnesyl-diphosphate farnesyltransferase 1 | RNA helicase-related protein |
| Interferon stimulated gene (20 kD) | |
| Steroid-5-alpha-reductase, alpha polypeptide 1 | |
| (3-oxo-5 alpha-steroid delta 4-dehydrogenase alpha 1) | |
| Prostaglandin-endoperoxide synthase 2 | |
| (prostaglandin G/H synthase and cyclooxygenase) | |
| Laminin, alpha 3 (nicein (150 kD), kalinin (165 kD), BM600 (150 kD), epilegrin) | |
| Collagen, type XVII, alpha 1 | Keratin 18 |
| Heparan sulfate (glucosamine) 3-O-sulfotransferase 1 | |
| Tubulin, alpha 2 | Adenylyl cyclase-associated protein |
| Forkhead box D1 | Cathepsin C |
| ESTs, Highly similar to AF151802_1 CGI-44 protein [H. sapiens] (T174688) | |
| Ribonucleotide reductase M2 polypeptide | |
| Laminin, gamma 2 (nicein (100 kD), kalinin (105 kD), | |
| BM600 (100 kD), Herlitz junctional epidermolysis bullosa)) | |
| Homo sapiens mRNA; cDNA DKFZp586P1622 (from clone DKFZp586P1622) | |
| ESTs, Weakly similar to/prediction (AA284245) | |
| Lactate dehydrogenase A | |

Note that in the parantheses after “EST(s)” is GENABNK ACESSION NO.

These genes, and others, are involved in the normal functioning of respiration as well as in diseases associated with respiratory pathologies, including cystic fibrosis, asthma, pulmonary hypertension and vasoconstriction, chronic obstructive pulmonary disease (COPD), pulmonary transplantation rejection, pulmonary infections, chronic bronchitis, respiratory distress syndrome (ARDS), allergic rhinitis, lung cancer and lung metastatic cancers and other airway diseases, including those with inflammatory response.

Anti-sense oligos to the target receptors, e.g. the adenosine A₁, A₂ₐ, A_{2b}, and A₃ receptors, CCR3 (chemokine receptors), bradykinin 2B, VCAM (vascular cell adhesion molecule), and eosinophil receptors, among others, have been shown to be effective in down-regulating the expression of their genes. Some of these act to alleviate the symptoms or reduce respiratory ailments and/or inflammation, for example, by “down regulation” of the adenosine A₁, A₂ₐ, A_{2b}, and/or A₃ receptors and CCR3, bradykinin 2B, VCAM (vascular cell adhesion molecule) and eosinophil receptors. These agents may be utilized by the present method alone or in conjunction with anti-sense oligos targeted to other genes to validate pathway and/or networks in which they are involved. For better results, the oligos are preferably administered directly into the respiratory system, e.g., by inhalation or other means, of the experimental animal, so that they may reach the lungs without widespread systemic dissemination. This permits the use of low agent doses as compared with those administered systemically or by other generalized routes and, consequently, reduces the number and degree of undesirable side effects resulting from the agent's widespread distribution in the body. The agent(s) of this invention has (have) been shown to reduce the amount of receptor protein expressed by the tissue. These agents, thus, rather than merely interacting with their targets, e.g. a receptor, lower the number of target proteins that other drugs may interact with. In this manner, the present agent(s) afford(s) extremely high efficacy with low toxicity. Anti-sense oligonucleotides to the A₁, A_{2b}, A₃, bradykinin B2, GATA-3, VCAM (vascular cell adhesion molecule), eosinophil receptors, and COX-2 receptors, among others, have been shown to be effective in the down-regulation of the respective receptor proteins in the cell. One novel feature of this treatment, as compared to traditional treatments for adenosine-mediated bronchoconstriction, is that administration is direct to the lungs, or in situ to other tissues, organs or systems of the body. Additionally, a receptor protein itself is reduced in amount, rather than merely interacting with a drug, and toxicity is reduced. Other proteins that may be targeted with anti-sense agents for the treatment of lung conditions include, but are not limited to: CCR3 (chemokine) receptors, human A₂ₐ adenosine receptor, human A_{2b} adenosine receptor, human IgE receptor β, human Fc-epsilon receptor CD23 antigen, human histidine decarboxylase, human beta tryptase, human tryptase-I, human prostaglandin D synthase, human cyclooxigenase-2, human eosinophil cationic protein, human eosinophil derived neurotoxin, human eosinophil peroxidase, human intercellular adhesion molecule-1 (ICAM-1), human vascular cell adhesion molecule-1 (VCAM-1), human endothelial leukocyte adhesion molecule-1 (ELAM-1), human P selectin, human endothelial monocyte activating factor, human IL-3, human IL-4, human IL-5, human IL-6, human IL-8, human monocyte-derived neutrophil chemotactic factor, human neutrophil elastase, human neutrophil oxidase factor, human cathepsin G, human defensin 1, human defensin 3, human macrophage inflammatory protein-1-alpha, human muscarinic acetylcholine receptor HM3, human fibronectin, human GM-CSF, human tumor necrosis factor α, human leukotriene C4 synthase, hum major basic protein, and human endothelin 1. Although not intended to be exclusive, a more extensive list of genes and sequences are provided below. Some of these act to alleviate the symptoms or reduce respiratory ailments and/or inflammation, for example, by “down regulation” of the adenosine A₁, A₂ₐ, A_{2b}, and/or A₃ receptors and CCR3, bradykinin 2B, VCAM (vascular cell adhesion molecule) and eosinophil receptors. These agents are preferably administered directly into the respiratory system, e.g., by inhalation or other means, so that they may reach the lungs without widespread systemic dissemination. This permits the use of substantially lower doses of the agent of the invention as compared with those administered by the prior art, systemically or by other generalized routes and, consequently, reduce undesirable side effects resulting from the agent's widespread distribution in the body. The agent(s) of this invention has (have) been shown to reduce the amount of receptor protein expressed by the tissue. These agents, thus, rather than merely interacting with their targets, e.g. a receptor, lower the number of target proteins that other drugs may interact with. In this manner, the present agent(s) afford(s) extremely high efficacy with low toxicity. In these latter targets, and in target genes in general, it is particularly imperative to eliminate or reduce the adenosine content of the corresponding anti-sense oligonucleotide to prevent their breakdown products from liberating adenosine.

As used herein, the term “treat” or “treating” refers to a treatment which decreases the likelihood that the subject administered such treatment will manifest symptoms of the respiratory, lung or other diseases. The term “downregulate” refers to inducing a decrease in production, secretion or availability (and thus a decrease in concentration) of the targeted intracellular protein. The present invention is concerned primarily with the treatment of human subjects. However, the agents and methods disclosed here may also be employed for veterinary purposes, such as is the case in the treatment of other mammals, such as cattle, horses, wild animals, zoo animals, and domestic animals, e.g. dogs and cats. Targeted proteins may be prokaryotic or eukaryotic or mammalian and more preferably of the same species as the subject being treated. In general, “anti-sense” refers to the use of small, synthetic oligonucleotides, resembling single-stranded DNA, to inhibit gene expression by inhibiting the function of the target messenger RNA (mRNA). Milligan, J. F. et al., S. Med. Chem. 36(14), 1923-1937 (1993). In the present invention, inhibition of gene expression of the A₁ or A₃ adenosine receptor is desired. Gene expression is inhibited through hybridization to coding (sense) sequences in a specific messenger RNA (mRNA) target by hydrogen bonding according to Watson-Crick base pairing rules. The mechanism of anti-sense inhibition is that the exogenously applied oligonucleotides decrease the mRNA and protein levels of the target gene or cause changes in the growth characteristics or shapes of the cells. Id. See, also Helene, C. and Toulme, J., Biochim. Biophys. Acta 1049, 99-125 (1990); Cohen, J. S. D., Ed., Oligodeoxynucleotides as Anti-sense Inhibitors of Gene Expression; CRC Press: Boca Raton, Fla. (1987). As used herein, “anti-sense oligonucleotide” is defined as a short sequence of synthetic nucleotide that (1) hybridizes to any sense or anti-sense sequence in a mRNA or DNA which codes for the targeted protein or their double stranded counterparts, according to in vitro or in vivo hybridization conditions, described below, and (2) upon hybridization causes a decrease in gene expression of the target, e.g. adenosine or other receptor(s). The receptors discussed above are mere examples of the high power of the present technology. In fact, a large number of genes and mRNAs may be targeted in a similar manner by the present methods, to significantly down-regulate or obliterate their protein expression and observe any changes wrought to one or more functions within a system e.g. the respiratory system and other lung disease associated targets. By means of example, in the respiratory system, the targets may be associated with difficulties of breathing, bronchoconstriction, inflammation, allergic rhinitis, chronic bronchitis, surfactant depletion, and others associated with diseases and conditions such as chronic obstructive pulmonary disease (COPD), pulmonary transplantation rejection, pulmonary infections, inhalation burns, Acute Respiratory Distress Syndrome (ARDS), cystic fibrosis, pulmonary fibrosis, radiation pulmonitis, tonsilitis, emphysema, dental pain oral inflammation, joint pain, esophagitis, cancers afflicting the respiratory system either directly such as lung cancer, esophageal cancer, and the like, or indirectly by means of metastases, among others. These functions are of great interest because of their association with respiratory dysfunction, as is the case in asthma, allergies, allergic rhinitis, pulmonary bronchoconstriction and hypertension, chronic obstructive pulmonary disease (COPD), pulmonary transplantation rejection, pulmonary infections, allergy, asthma, cystic fibrosis (CF), Acute Respiratory Distress Syndrome (ARDS) as well as infantile and pregnancy-related RDS, cancer, etc., which either directly or by metastasis afflict the lung, the present anti-sense oligonucleotides may be directed to a list of target mRNAs, which includes the targets listed in Table 1 above, among others.

Oligonucleotides, whether DNA or RNA, may be synthesized by methods known in the art that need not be further described here. The low adenosine oligos of this invention may be obtained by first selecting fragments of a target nucleic acid having at least 4 contiguous nucleic acids selected from the group consisting of G and C and/or having a specific type and/or extent of activity, and then obtaining a first oligonucleotide 4 to 60 nucleotides long which comprises the selected fragment and has a thymidine (T) or uridine (U) nucleic acid content of up to and including about 15%, preferably, about 12%, about 10%, about 7%, about 5%, about 3%, about 1%, and more preferably no thymidine or uridine. In one preferred embodiment, oligo(s) have a higher than natural content of Cs and Gs (or CpGs) to produce immunostimulation. The latter step may be conducted by obtaining a second oligonucleotide 4 to 60 nucleotides long comprising a sequence which is anti-sense to the selected fragment, the second oligonucleotide having an adenosine base content of up to and including about 15%, preferably about 12%, about 10%, about 7%, about 5%, about 3%, about 1%, and more preferably no adenosine. When the selected fragment comprises at least one thymidine or uridine base, an adenosine base may be substituted in the corresponding anti-sense nucleotide fragment with a universal base selected from the group consisting of heteroaromatic bases which bind to a thymidine or uridine base but have less than about bout 10%, preferably less than about 1%, and more preferably less than about 0.3% of the adenosine base agonist activity at the adenosine A₁, A₂ₐ, A_{2b} and A₃ receptors, and heteroaromatic bases which have no activity at the adenosine A₂ₐ receptor, when validating in the respiratory system. Other adenosine activities in other systems may be determined in other systems, as appropriate. The analogue heteroaromatic bases may be selected from all pyrimidines and purines, which may be substituted by O, halo, NH₂, SH, SO, SO₂, SO₃, COOH and branched and fused primary and secondary amino, alkyl alkenyl, alkynyl, cycloalkyl, heterocycloalkyl, aryl, heteroaryl alkoxy, alkenoxy, acyl, cycloacyl, arylacyl, alkynoxy, cycloalkoxy, aroyl arylthio, arylsulfoxyl, halocycloalkyl, alkylcycloalkyl, alkenylcycloalkyl, alkynylcycloalkyl, haloaryl, alkylaryl, alkenylaryl alkynylaryl, arylalkyl, arylalkenyl, arylalkynyl arylcycloalkyl, which may be further substituted by O, halo, NH₂, primary, secondary and tertiary amine, SH, SO, SO₂, SO₃, cycloalkyl, heterocycloalkyl and heteroaryl. The pyrimidines and purines may be substituted at all positions as is known in the art, but preferred are purines that are substituted at positions 1, 2, 3, 6 and/or 8, and pyrimidines that are substituted at 2, 3, 4, 5 and/or 6. More preferred are pyrimidines and purines such as those having the chemical formula 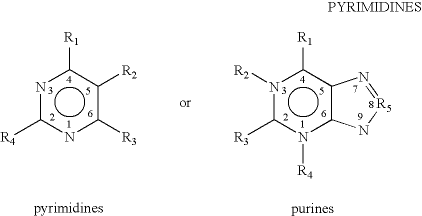
wherein R¹, R², R³, R⁴, and R⁵ are independently H, alkyl, alkenyl or alkynyl and R³ is H, aryl, dicycloalkyl, dicycloalkenyl, dicycloalkynyl, cycloalkyl, cycloalkenyl, cycloalkynyl, O-cycloalkyl, O-cycloalkenyl, O-cycloalkynyl, NH₂-alkylamino-ketoxyalkyloxy-aryl, or mono or dialkylaminoalkyl-N-alkylamino-SO₂aryl, and R4 and R5 are independently R1 and together are R3, and the pyrimidines and purines optionally comprise theophylline, caffeine, dyphylline, etophylline, acephylline piperazine, bamifylline, enprofylline or xantine, among others. Similar modifications in the sugar are also embodiments of this invention. Reduced adenosine content of the anti-sense oligos corresponding to the thymidines (T) present in the target DNA or uridines (U) in the target RNA serves to prevent the breakdown of the oligos into products that free adenosine into the system, e.g. the lung, brain, heart, kidney, etc., tissue environment and, thereby, to prevent any unwanted effects due to it. By means of example, the NfκB transcription factor may be selected as a target, and its mRNA or DNA searched for low thymidine (T), low uridine (U) or desthymidine (desT) or desuridine (desU) fragments. Only desU and desT segments of the mRNA or DNA are selected which, in turn, will produce desA anti-sense as their complementary strand. When a number of DNA or RNA that are desT or desU segments are found, the sequence of the anti-sense segments may be deduced. Typically, about 10 to 30 and even larger numbers of desA anti-sense sequences may be obtained. These anti-sense sequences may include some or all desA anti-sense oligonucleotide sequences corresponding to desU or desT segments of the mRNA or DNA of the target, such as anyone of those shown in Table 1 above, in Table 2 below, and others associated with functions of the brain, cardiovascular and renal systems, and many others. For each of the original desA anti-sense oligonucleotide sequences corresponding to the target gene, e.g. the NFκB transcription factor, typically about 10 to 30 sequences may be found within the target gene or RNA which have a low content of thymidine (DNA) or uridine (RNA). In accordance with this invention, the selected fragment sequences may also contain a small number of thymidine (DNA) or uridine (RNA) nucleotides within the secondary or tertiary or quaternary sequences. In some cases, a large adenosine content may suffice to render the anti-sense oligonucleotide less active or even inactive against the target. In accordance with this invention, these so called “non-fully desA” sequences may preferably have a content of adenosine of less than about 15%, about 12%, about 10%, about 7%, about 5%, and about 2% adenosine. Most preferred is no adenosine content (0%). In some instances, however, a higher content of adenosine is acceptable and the oligonucleotides still fail to show detrimental “adenosine activity”. A particular important embodiment is that where the adenosine nucleotide is “fixed” or replaced by a “universal or alternative” base that may base-pair with similar or equal affinity to two or more of the four nucleotides present in natural DNA: A, G, C, and T.

A universal or alternative base is defined in this patent as any compound, more commonly an adenosine analogue, which has substantial capacity to hybridize to thymidine or uridine, while at the same time having reduced, or substantially lacking, ability to bind adenosine receptors or other molecules through which adenosine may exert an undesirable side effect in the experimental animal or in a cell system. Alternatively, adenosine analogs which completely fail to activate, or have significantly reduce ability for activating, adenosine receptors, such as the adenosine A₁, A_{2b} and/or A₃ receptors, most preferably A₁ receptors, and those that may even act as agonists of the adenosine A₂ₐ, receptor, may be used. One example of a universal base is 2′-deoxyribofuranosyl-(5-nitroindole), and an artisan will know how to select others. This “fixing” step generates further novel sequences, different from those anti-sense to the ones found in nature, that permits the anti-sense oligonucleotide to bind, preferably equally well, with the target RNA. Other examples of universal or alternative bases are 2′-deoxyribosyl-(5-nitroindole). Other examples of universal bases are 3-nitropyrrole-2′-deoxynucleoside, 5-nitro-indole, 2′-deoxyribosyl-(5-nitroindole), 2′-deoxyribofuranosyl-(5-nitroindole), 2′-deoxyinosine, 2′-deoxynebularine, 6H, 8H-3,4-dihydropyrimido[4,5-c]oxazine-7-one and 2-amino-6-methoxy aminopurine. In addition to the above, Universal bases which may be substituted for any other base although with somewhat reduced hybridization potential, include 3-nitropyrrole-2′-deoxynucleoside 2′-deoxyribofuranosyl-(5-nitroindole), 2′-deoxyinosine and 2′-deoxynebularine (Glen Research, Sterling, Va.). More specific mismatch repairs may be made using “P” nucleotide, 6H, 8H-3,4-dihydropyrimido[4,5-c][1, 2]oxazin-7-one, which base pairs with either guanosine (G) or adenosine (A) and “K” nucleotide, 2-amino-6-methoxyaminopurine, which base pairs with either cytidine (C) or thymidine (T)-uridine (U), among others. Others that are known in the art or will become available are also suitable. See, for example, Loakes, D. and Brown, D. M., Nucl. Acids Res. 22:4039-4043 (1994); Ohtsuka, E. et al., J. Biol. Chem. 260(5):2605-2608 (1985); Lin, P.K.T. and Brown, D. M., Nucleic Acids Res. 20(19):5149-5152 (1992; Nichols, R et al., Nature 369(6480): 492-493 (1994); Rahmon, M. S. and Humayun, N. Z., Mutation Research 377 (2): 263-8 (1997); Amosova, O., et al., Nucleic Acids Res. 25 (10): 1930-1934 (1997); Loakes D. & Brown, D. M., Nucleic Acids Res. 22 (20): 4039-4043 (1994), the entire sections relating to universal bases and their preparation and use in nucleic acid binding being incorporated herein by reference. When non-fully desT sequences are found in the naturally occurring target, they typically are selected so that about 1 to 3 universal base substitutions will suffice to obtain a 100% “desA” anti-sense oligonucleotide. Thus, the present method provides either anti-sense oligonucleotides to different targets which are low in, or devoid of, A content, as well as anti-sense oligonucleotides where one or more adenosine nucleotides, e.g. about 1 to 3, or more, may be “fixed” by replacement with a universal” or “replacement” base. Universal bases are known in the art and need not be listed herein. An artisan will know which bases may act as universal bases, and replace them for A. Table 2 below provides a selected number of targets to which the agents of the invention are effectively applied. Others, however, may also be targeted.

TABLE 2
Cancer Targets

| Transforming | Therapy |
|---|---|
| Oncogenes | Targets |
| ras | thymidylate synthetase |
| src | thymidylate synthetase |
| myc | dihydrofolate reductase |
| bcl-2 | thymidine kinase |
| | deoxycytidine kinase |
| | ribonucleotide reductase |
| Angiogenesis factors | Adhesion Molecules |
| Oncogenes | Folate Pathway Enzymes |
| DNA repair genes | (One Carbon Pool) |
| | Telomerase |
| | HMG CoA Reductase |
| | Farnesyl Transferase |
| | Glucose-6-Phosphate Transferase |
| | Akt2 (Bases 1-1715) |
| Akt3 (1-1547) | |
| Ampiregulin (1-1230)) | |
| Ap-2 (1-1391) | |
| Ap-2 Beta | |
| Ap-2 Gamma | |
| Sphingomyelinase | |
| Beta-2-Adernergic Receptor | |
| Beta Catenin | |
| E2F-Related Transcription Factor | |
| HM bFGF | |
| B-cell translocation gene 1 (BTG1) | |
| cyclin-dependent kinase 2 (CDK2) | |
| cyclin-dependent kinase 2 (CDK2) | |
| cyclin-dependent kinase 3 (CDK3) | |
| cyclin-dependent kinase 4 (CDK4) | |
| cyclin-dependent kinase 5 (CDK5) | |
| c-ets-1 proto-oncogene | |
| checkpoint kinase Chk1 (CHK1) | |
| type IV collagenase | |
| hepatocyte growth factor | |
| receptor (c-met) | |
| MYB proto-oncogene protein (MYB) | |

A group of preferred targets for the treatment of cancer are genes associated with any of different types of cancers, or those generally known to be associated with malignancies, whether they are regulatory or involved in the production of RNA and/or proteins. Examples are transforming oncogenes, including, but not limited to, ras, src, myc, and BCL-2, among others. Other targets are those to which present cancer chemotherapeutic agents are directed to, such as various enzymes, primarily, although not exclusively, thymidylate synthetase, dihydrofolate reductase, thymidine kinase, deoxycytidine kinase, ribonucleotide reductase, and the like. The present technology is particularly useful in the treatment of cancer ailments given that traditional cancer therapies are fraught with the unresolved problem of selectively killing cancer cells while preserving normal living cells from the devastating effects of treatments such as chemotherapy, radiotherapy, and the like. The present technology provides the ability of selectively attenuating or enhancing a desired pathway or target. This approach provides a significant advantage over standard treatments of cancer because it permits the selection of a pathway, including primary, secondary and possibly tertiary targets, which are not generally expressed simultaneously in normal cells. Thus, the present agent may be administered to a subject to cause a selective increase in toxicity within tumor cells that, for instance, express all three targets while normal cells that may expresses only one or two of the targets will be significantly less affected or even spared. A group of preferred targets for the treatment of cancers are genes associated with different types of cancers, or those generally known to be associated with malignancies, whether they are regulatory or involved in the production of RNA and/or proteins. Examples are transforming oncogenes, including, but not limited to, ras, src, myc, and BCL-2, among others. Other targets are those to which present cancer chemotherapeutic agents are directed to, such as various enzymes, Primarily, although not exclusively, thymidylate synthetase, dihydrofolate reductase, thymidine kinase, deoxycytidine kinase, ribonucleotide reductase, and the like.

In one embodiment, at least one of the genes or mRNAs to which the oligo of the invention is targeted encodes or is involved in the regulation of a protein such as transcription factors, stimulating and activating factors, intracellular and extracellular receptors and peptide transmitters in general, interleukins, interleukin receptors, chemokines, chemokine receptors, endogenously produced specific and non-specific enzymes, immunoglobulins, antibody receptors, central nervous system (CNS) and peripheral nervous and non-nervous system receptors, CNS and peripheral nervous and non-nervous system peptide transmitters, adhesion molecules, defensines, growth factors, vasoactive peptides and receptors, and binding proteins, among others; or the mRNA is corresponding to an oncogene and other genes associated with various diseases or conditions. Examples of target proteins are eotaxin, major basic protein, preproendothelin, eosinophil cationic protein, P-selectin, STAT 4, MIP-1α, MCP-2, MCP-3, MCPA-4, STAT 6, c-mas, NF-IL-6, cyclophillins, PDG2, cyclosporin A-binding protein, FK5-binding protein, fibronectin, LFA-1 (CD11a/CD18), PECAM-1, C3bi, PSGL-1,CD-34, substance P, p150,95, Mac-1 (CD11b/CD18), VLA-4, CD-18/CD11a, CD11b/CD18, C5a, CCR1, CCR2, CCR4, CCR5, and LTB-4, among others. Others are, however, suitable, as well. In another embodiment, at least one of the mRNAs to which the oligo is targeted encodes intracellular and extracellular receptors and peptide transmitters such as sympathomimetic receptors, parasympathetic receptors, GABA receptors, adenosine receptors, bradykinin receptors, insulin receptors, glucagon receptors, prostaglandin receptors, thyroid receptors, androgen receptors, anabolic receptors, estrogen receptors, progesterone receptors, receptors associated with the coagulation cascade, adenohypophyseal receptors, adenohypophyseal peptide transmitters, and histamine receptors (HisR), among others. However others are also contemplated. The encoded sympathomimetic receptors and parasympathonimetic receptors include acetylcholinesterase receptors (AcChaseR) acetylcholine receptors (AcChR), atropine receptors, muscarinic receptors, epinephrine receptors (EpiR), dopamine receptors (DOPAR), and norepinephrine receptors (NEpiR), among others. Further examples of encoded receptors are adenosine A₁ receptor, adenosine A_{2b} receptor, adenosine A₃ receptor, endothelin receptor A, endothelin receptor B, IgE high affinity receptor, muscarinic acetylcholine receptors, substance P receptor, histamine receptor, CCR-1 CC chemokine receptor, CCR-2 CC chemokine receptor, CCR-3 CC chemokine receptor (Eotaxin Receptor), interleukin-1β receptor (IL-1βR), interleukin-1 receptor (IL-1R), interleukin-1β receptor (IL-1βR), interleukin-3 receptor (IL-3R), CCR-4 CC chemokine receptor, cysteinyl leukotriene receptors, prostanoid receptors, GATA-3 transcription factor receptor, interleukin-1 receptor (IL-1R), interleukin-4 receptor (II-4R), interleukin-5 receptor (IL-5R), interleukin-8 receptor (IL-8R), interleukin-9 receptor (IL-9R), interleukin-11 receptor (IL-11R), sympathomimetic receptors, parasympathomimetic receptors, GABA receptors, adenosine receptors, bradykinin receptors, e.g. bradykinin B2 receptor, insulin receptors, glucagon receptors, prostaglandin receptors, thyroid receptors, androgen receptors, anabolic receptors, estrogen receptors, progesterone receptors, receptors associated with the coagulation cascade, adenohypophyseal receptors, and histamine receptors (HisR). Others are also contemplated even though not listed herein. The encoded enzymes for development of the oligos of the invention include synthetases, kinases, oxidases, phosphatases, reductases, polysaccharide, triglyceride, and protein hydrolases, esterases, elastases, and, polysaccharide, triglyceride, lipid, and protein synthases, among others. Examples of target enzymes are tryptase, inducible nitric oxide synthase, cyclooxygenase (Cox), MAP kinase, eosinophil peroxidase, β2-adrenergic receptor kinase, leukotriene c-4 synthase, 5-lipooxygenase, phosphodiesterase IV, metalloproteinase, tryptase, CSBP/p38 MAP kinase, neutrophil elastase, phospholipase A₂, cyclooxygenase 2 (Cox-2), fucosyl transferase, chymase, protein kinase C, thymidylate synthetase, dihydrofolate reductase, thymidine kinase, deoxycytidine kinase, and ribonucleotide reductase, among others. Any enzyme associated with a disease or condition, however, is suitable as a target for this invention. Suitable encoded factors for application of this invention are, among others, NfκB transcription factor, granulocyte macrophage colony stimulating factor (GM-CSF), AP-1 transcription factor, GATA-3 transcription factor, monocyte activating factor, neutrophil chemotactic factor, granulocyte/macrophage colony-stimulating-factor (G-CSF), NFAT transcription factors, platelet activating factor, tumor necrosis factor α (TNF α), and basic fibroblast growth factor (BFGF). Additional factors are also within the invention even though not specifically mentioned. Suitable adhesion molecules for use with this invention include intracellular adhesion molecules 1 (ICAM-1), 2 (ICAM-2) and 3 (ICAM-3), vascular cellular adhesion molecule (VCAM), endothelial leukocyte adhesion molecule-1 (ELAM-1), neutrophil adherence receptor, and CAM-1, and the like. Other known and unknown factors (at this time) may also be targeted herein. Among the cytokines, lymphokines and chemokines preferred are interleukin-1 (IL-1), interleukin-1β (IL-1β), interleukin-3 (IL-3), interleukin-4 (IL-4), interleukin-5 (IL-5), interleukin-8 (IL-8), interleukin-9 (IL-9), interleukin-11 (IL-11), CCR-5 CC chemokine, and Rantes. Other examples include H2A histone family, member N, Tubulin, beta polypeptide, ELL gene (11-19 lysine-rich leukemia gene) 7-dehydrocholesterol reductase, ADP-ribosylation factor-like 7, Karyopherin alpha 2 (RAG cohort 1, importin alpha 1), EST (AI038433), EST (AI122689), EST (AI092623), ESTs (AI095492), ESTs (AI138216), ESTs (AI128305), ESTs (AI125228), ESTs (AI041482), ESTs (AI051839), Homo sapiens mRNA; cDNA DKFZp434A1716, ESTs (AI096522), ESTs (AI122807), ESTs (AI041212), EST (AI125651), Enolase 1, (alpha), EST (AI024215), EST (AI034360), Homo sapiens mRNA; cDNA DKFZp564H0764, Homo sapiens mRNA for KIAA1363 protein, partial cds, Potassium voltage-gated channel, shaker-related subfamily, beta member 2, ER-associated DNAJ; ER-associated Hsp40 co-chaperone; hDj9; ERj3, ESTs, Weakly similar to p38 protein [H. sapiens] (AA906703), CGI-142, ESTs (AA463249), Homo sapiens clone 25058 rRNA sequence ESTs (R49144), Squamous cell carcinoma antigen 1, ESTs (AA425700), Myosin X, ESTs (AA459692), Epithelial protein lost in neoplasm beta, CD44 antigen (homing function and Indian blood group system), Coagulation factor III (thromboplastin, tissue factor), ESTs (AA909635), Adducin 1 (alpha), 5′ Nucleotidase (CD73), ESTs, Moderately similar to semaphorin C [M. musculus] (AA293300), ESTs (AA278764), ESTs (AA678160), Calmodulin 2 (phosphorylase kinase, delta), ESTs (R42770), Chloride intracellular channel 1, High-mobility group (nonhistone chromosomal) protein 17, Ubiquitin carrier protein, Tubulin alpha 1 (testis specific), Transglutaminase 2 (C polypeptide, protein-glutamine-gamma-glutamyltransferase), Sparc/osteonectin, cwcv and kazal-like domains proteoglycan (testican), Proteasome (prosome, macropain) 26S subunit, non-ATPase, 2, Tubulin beta polypeptide, Filamin B, beta (actin-binding protein-278), Stanniocalcin, Low density lipoprotein receptor (familial hypercholesterolemia), Plectin 1, intermediate filament binding protein, 500 kD, S100 calcium-binding protein A2, Immediate early response 3, Calpain, large polypeptide L2, Pleckstrin homology-like domain, family A, member 1, Melanoma adhesion molecule, CD44 antigen (homing function and Indian blood group system), Programmed cell death 5, Hexokinase 1, Vascular endothelial growth factor, Integrin, alpha 2 (CD49B, alpha 2 subunit of VLA-2 receptor), Calumenin, Syntaxin 11, Diphtheria toxin receptor (heparin-binding epidermal growth factor-like growth factor), Fn14 for type I transmenmbrane protein, Nef-associated factor 1, High-mobility group (nonhistone chromosomal) protein isoforms I and Y, Catechol-O-methyltransferase, C-terminal binding protein 1, Collagen, type XVII, alpha 1, ESTs (N58473), Farnesyl-diphosphate farnesyltransferase 1 RNA helicase-related protein, Interferon stimulated gene (20 kD), Steroid-5-alpha-reductase, alpha polypeptide 1 (3-oxo-5 alpha-steroid delta 4-dehydrogenase alpha 1), Prostaglandin-endoperoxide synthase 2 (prostaglandin G/H synthase and cyclooxygenase), Laminin, alpha 3 (nicein (150 kD), kalinin (165 kD), BM600 (150 kD), epilegrin), Collagen, type XVII, alpha 1, Keratin 18, Heparan sulfate (glucosamine) 3-O-sulfotransferase 1, Tubulin, alpha 2, Adenylyl cyclase-associated protein, Forkhead box D1, Cathepsin C, ESTs, Highly similar to AF151802_{—}1 CGI-44 protein [H. sapiens] (T74688), Ribonucleotide reductase M2 polypeptide, Laminin, gamma 2 (nicein (100 kD), kalinin (105 kD), BM600 (100 kD), Herlitz junctional epidermolysis bullosa)), Homo sapiens mRNA; cDNA DKFZp586P1622 (from clone DKFZp586P1622), ESTs, Weakly similar to/prediction (AA284245), and Lactate dehydrogenase A. Others, however, may also be targeted, as they are known to be involved in specific diseases or conditions to be treated, or for their generic activities, such as inflammation. Examples of defensins for the practice of this invention are defensin 1, defensin 2, and defensin 3, and of selectins are α4β1 selectin, α4β7 selectin, LFA-1 selectin, E-selectin, P-selectin, and L-selectin. Examples of oncogenes, although not an all inclusive list, are ras, src, myc, and bcBCL. Others, however, are also suitable for use with this invention.

The agents administered in accordance with this invention are preferably designed to be anti-sense to one or more target genes and/or mRNAs usually related in origin to the species to which it is to be administered, although they may be directed, to foreign sequences, e.g. of viruses. When treating humans, the agents are preferably designed to be anti-sense to a human gene or RNA. The agents of the invention encompass oligonucleotides which are anti-sense to naturally occurring DNA and/or RNA sequences, fragments thereof of up to a length of one (1) base less than the targeted sequence, preferably at least about 7 nucleotides long, oligos having only over about 0.02%, more preferably over about 0.1%, still more preferably over about 1%, and even more preferably over about 4% adenosine nucleotides, and up to about 30%, more preferably up to about 15%, still more preferably up to about 10% and even more preferably up to about 5%, adenosine nucleotide, or lacking adenosine altogether, and oligos in which one or more of the adenosine nucleotides have been replaced with so-called universal bases, which may pair up with thymidine or uridine nucleotides but fail to substantially trigger adenosine receptor activity. Examples of human sequences and fragments, which are not limiting, of anti-sense oligonucleotide of the invention are the following fragments as well as shorter segments of the fragments and of the full gene or mRNA coding sequences, exons and intron-exon junctions encompassing preferably 7, 10, 15, 18 to 21, 24, 27, 30, n−1 nucleotides for each sequence, where n is the sequence's total number of nucleotides. These fragments may be selected from any portion of the longer oligo, for example, from the middle, 5′-end, 3′-end or starting at any other site of the original sequence. Of particular importance are fragments of low adenosine nucleotide content, that is, those fragments containing less than or about 30%, preferably less than or about 15%, more preferably less than or about 10%, and even more preferably less than or about 5%, and most preferably those devoid of adenosine nucleotide, either by choice or by replacement with a universal base in accordance with this invention. The agent of the invention includes as a most preferred group sequences and their fragments where one or more adenosines present in the sequence have been replaced by a universal base (B), as exemplified here. Similarly, also encompassed are all shorter fragments of the B-containing fragments designed by substitution of B(s) for adenosine(s) (A(s)) contained in the sequences, fragments thereof or segments thereof, as described above. A limited list of sequences and fragments is provided below.

Some of the examples of anti-sense oligonucleotide sequence fragments target the initiation codon of the respective gene, and in some cases adenosine is substituted with a universal or alternative base adenosine analogue denoted as “B”, which lacks ability to bind to the adenosine A₁ and/or A₃ receptors. In fact, such replacement nucleotide acts as a “spacer”. Many of the examples shown below provide one such sequence and many fragments overlapping the initiation codon, preferably wherein the number of nucleotides n is about 7, about 10, about 12, about 15, about 18, about 21 and up to about 28, about 35, about 40, about 50, about 60.

LENGTHY TABLE REFERENCED HERE
US20070021360A1-20070125-T00001
Please refer to the end of the specification for access instructions.

In one preferred embodiment, the links between neighboring mononucleotides are phosphodiester links. In another preferred, at least one mononucleotide phosphodiester residue of the anti-sense oligonucleotide(s) is substituted by a methylphosphonate, phosphotriester, phosphorothioate, phosphorodithioate, boranophosphate, formacetal, thioformacetal, thioether, carbonate, carbamate, sulfate, sulfonate, sulfamate, sulfonamide, sulfone, sulfite, sulfoxide, sulfide, hydroxylamine, 2′-O-methyl, methylene(methylmino), methyleneoxy(methylimino), phosphoramidate residues, and combinations thereof. The oligos having one or more phosphodiester residues substituted by one or more of the other residues are generally longer lasting, given that these residues are more resistant to hydrolysis than the phosphodiester residue. In some cases up to about 10%, about 30%, about 50%, about 75%, and even all phosphodiester residues may be substituted (100%).

In another preferred embodiment, the multiple target anti-sense oligo (MTA) of the invention comprises at least about 7 mononucleotides, in some instances up to 60 and more mononucleotides, preferably about 10 to about 36, and more preferably about 12 to about 21 mononucleotides. However, other lengths are also suitable depending on the length of the target macromolecule. Examples of multi-targeted anti-sense (MTA) oligos of the invention are provided in Table 3 below, which includes ninety-four sequences (SEQ ID NOS.: 2316 through 2410).

TABLE 3
MTA Oligos, Location Targeted & Target

| | | | | |
|---|---|---|---|---|
| | SEQ. | | | |
| | ID | | Compound | |
| MTA Oligo | No. | Location | Targeted | Target |

HUMNFKBP65A AS

| | | | | |
|---|---|---|---|---|
| CCC GGC CCC GCC TCG TGC C | 12388 | 5′ = 1 | EPI 2192 | |
| CGT CCB TGC CGC GGG CCC | 12389 | 5′ = 28 (AUG) | EPI 2193 | |
| GCC CCG CTG CTT GGG CTG CTC TGC CGG G | 12390 | 5′ = 65 | EPI 2194 | |
| TCT GTG CTC CTC TCG CCT GGG | 12391 | 5′ = 137 | EPI 2195 | |
| TGG TGG GGT GGG TCT TGG TGG | 12392 | 5′ = 159 | EPI 2196 | |
| CTG TCC CTG GTC CTG TG | 12393 | 5′ = 196 | EPI 2197 | |
| GGT CCC GCT TCT TC | 12394 | 5′ = 362 | EPI 2198 | |
| GGG GTT GTT GTT GGT CTG G | 12395 | 5′ = 401 | EPI 2199 | |
| TGT CCT CTT TCT GC | 12396 | 5′ = 656 | EPI 2200 | |
| GCC TCG GGC CTC CC | 12397 | 5′ = 697 | EPI 2201 | |
| GGC TGG GGT CTG CGT | 12398 | 5′ = 769 | EPI 2202 | |
| GGC CGG GGG TCG GTG GGT CCG CTG | 12399 | 5′ = 953 | EPI 2203 | |
| GGG CTG GGG TGC TGG CTT GGG G | 12400 | 5′ = 1022 | EPI 2204 | |
| GGG GCT GGG GCC TGG GCC | 12401 | 5′ = 1208 | EPI 2205 | |
| GCC TGG GTG GGC TTG GGG GC | 12402 | 5′ = 1272 | EPI 2206 | |
| GCT GGG TCT GTG CTG TTG CC | 12403 | 5′ = 1362 | EPI 2207 | |
| GTT GTG TGG GGG GCC | 12404 | 5′ = 1451 | EPI 2208 | |
| GCT GGG TCG GGG GGC CTC TGG GCT GTC | 12405 | 5′ = 1511 | EPI 2209 | |
| GCC CCG GGG CCC CC | 12406 | 5′ = 1550 | EPI 2210 | |
| TGG CTC CCC CCT CC | 12407 | 5′ = 1772 | EPI 2211 | |
| GCT CCC CCC TTT CC | 12408 | 5′ = 1863 | EPI 2212 | |
| CGG ACG AAG ACA GAG A | 12409 | 5′ = 1979 | EPI 2213 | |
| GGC TTT GTG GGC TC | 12410 | 5′ = 2011 | EPI 2214 | |
| GCC TGC TCT CCC CC | 12411 | 5′ = 2312 | EPI 2215 | |
| CCC GGC CCC GCC BCG BBC C | 12412 | intron | EPI 2192-01A | HSUS0136C4Synth |
| CCC GGC CCC GCC BCG | 12413 | intron | EPI 2192-01B | |
| CCC GGC CCC GCC BCG BBC C | 12414 | 5′untr | EPI 2192-02A | HUMLIPOX5LO |
| CCC GGC CCC GCC BCG | 12415 | 5′untr | EPI 2192-02B | |
| CCC GBC CCC GCC TCB BG | 12416 | trans | EPI 2192-03A | HSNFKBS Subunit |
| CCC GBC CCC GCC TC | 12417 | trans | EPI 2192-03B | |
| CCG GCC CCG CCT C | 12418 | 5′untr | EPI 2192-04 | TGFβR1 |
| CCC GBB CCC GCB TBG TGC C | 12419 | 5′trans | EPI 2192-05A | HSUS8198I1 enhan |
| CCC GCB TBG TGC C | 12420 | 5′untr | EPI 2192-05B | |
| CCC GGB CCC BCC BBG TGC C | 12421 | 3′trans | EPI 2192-06 | HSVECAD |
| CBG BBC CCG CCT CGT GCC | 12422 | intron | EPI 2192-07A | NFKB2 |
| C CCG CCT CGT GCC | 12423 | intron | EPI 2192-07B | NFKB2 |
| CCG GCB CCG CCT CBT GCC | 12424 | 5′trans | EPI 2192-08 | Carboxypep |
| CCG GCC CCG CCB CBT GCC | 12425 | 3′trans | EPI 2192-09 | HumADRA2C⊕2AdrKid |
| CCC GBC CCC GBC TCG | 12426 | 5′untrs | EPI 2192-10 | HUMFK506B |
| CCC GGC CBC GBC TCG | 12427 | 5′untrs | EPI 2192-11 | HSNBARKS1βAdrKin |
| CCC GGC CCB GCC TBG | 12428 | 5′UTR | EPI 2192-12 | HSNFXN1 (NFKB1) |
| CCC GGC BCB GBC TCG TBC C | 12429 | 3′UTR | EPI 2192-13 | HSILF (transcrp. |
| | | | | Factor ILF) |
| CCC GGC CCC GCC BCG | 12413 | | EPI-2192-14 | NFKB/C4Syn/5-LO/ |
| | | | | TGFBrec1 MTA |
| CCC GGC CCC GCC BCG | 12430 | | EPI-2192-15 | NPKB/C4Syn/5-LOMTA |
| TCC BTG CCG CGG GC | 12432 | 3′trans | EPI-2193-01 | METOncogene |
| TCC BTG CCB CGG GCC | 12433 | 3′trans | EPI-2193-02 | HSFGR2 (IG) |
| TCC BTG CCB CGG GCC | 12434 | mid cod | EPI-2193-03 | 5-LO |
| TCC BTG CCB CBG GCC | 12435 | mid cod | EPI-2193-04 | HUMTK14 |
| GTC CBT GBC GCG G | 12436 | 3′trans | EPI-2193-05 | HUMTNFR |
| TC CBT GBC GCG GG | 12437 | AUG | | Probl.HUMPTCH |
| | | | | cardiacK + channel |
| TCT GBG CTC CTC TBB CCT GGG | 12438 | intr | EPI-2195-01 | humCSPAcytotox. |
| | | | | Ser. Protease |
| CTG TGC BCC TBB CBC CTG GG | 12439 | intr | EPI-2195-02 | HSINOSX08induc.NOS |
| TGT GBT CCB CTB GBC TGG G | 12440 | | EPI-2195-03 | HUMACHRM2musc.m2 |
| | | | | acetylch.rec. |
| TCT GTB CTC BBC TCB CCT G | 12441 | | EPI-2195-04 | s86371s1 |
| | | | | Neurokinin3Recept |
| TGC TCC TCB CBB CTG GG | 12442 | | EPI-2195-05 | HUMMIP1 Amacro |
| | | | | Inflam. Factor |
| CTC CTC TBG CCT GG | 12443 | | EPI-2195-06 | HSNBARKS4 |
| | | | | β-Adr Rec Kinase |
| GTG CTC CBB TCB BCT GGG | 12444 | | EPI-2195-07 | HSTNFR2SO6TNF R2 |
| GTG CBC CBB TCB CCT GGG | 12445 | | EPI-2195-08 | humfkbp fk506 |
| | | | | binding prot. |
| TCT GTG CBC CTC TBG BCT | 12446 | exon | EPI-2195-09 | HSNBARKS1β-Adr. |
| | | | | Recept. Kinase |
| CTG TBB TCC TBB CBC CTG G | 12482 | intron | EPI-2195-10 | HUMIL8 |
| TGT GCT BBT CBC BCB TGG G | 12448 | | EPI-2195-11 | HSU50157 PDE4 |
| GTG CBC CBC TCB CCT G | 12449 | intron/exon | EPI-2195-12 | IL-2 R |
| CTG TGC BCC TCT C | 12450 | 3′UTR | EPI-2203-05 | IL-6 R HSIL6R |
| CBG TGC BCC BCT CBC CTG | 12451 | intr/ex | EPI-2203-06A | HSIL2rG6 |
| G TGC BCC BCT CBC CTG | 12449 | intr/ex | EPI-2203-06B | HSIL2rG6 |
| CBC CTC TCB CCT GGG | 12453 | coding | EPI-2203-07A | HUMIL71 |
| C CTC TCB CCT GGG | 12454 | coding | EPI-2203-07B | IL-7 HUMIL71 |
| GCT CCB CTC GCC T | 12455 | coding | EPI-2203-08 | IL-6 R HSI6REC |
| TGC TCC TCB CGC C | 12456 | intron PDGF A | EPI-2303-09 | Chain HUMPDGFAB |
| GTT GTT GBT CTG G | 12457 | 3′utr | EPI-2199-01 | GATA-4Transcrip. |
| | | | | Factor for IL-5 |
| GGT TGB BBT TGG TCT TGG | 12458 | Coding | EPI-2199-02 | TNF⊕ HUMTNFA |
| GGT TGT TGB TGB TCT G | 12459 | Far 5′UTR | EPI-2199-03 | HSSUBP1G (Sub Pr) |
| GGG TTB BBG TTG BTC TGG | 12460 | Coding | EPI-2199-04 | NeutrophilAdh. |
| | | | | R HUMNARIA |
| GGG TTB BBG TTG BTC TGG | 12461 | HSHM2 | EPI-2199-05 | m2 Muscarinic R |
| TTG TTG TBG BTC TGG | 12462 | HUML1CAM | EPI-2199-06 | L1 LeukAadhProt |
| GGG TGB BBG BGT CCG CTG | 12463 | coding | EPI-2203-01 | HUMGATA2A |
| GGG TCB GBG GBT CBG CTG | 12464 | S71424S2 | EPI-2203-02 | IGE eps |
| GGG TGB GTG GGT C | 12465 | coding | EPI-2203-03 | HSGCSFR2 |
| GGG TCG GBG GGT CBG C | 12466 | HUMITGF | EPI-2203-04 | TGFβ3 |
| GGG TGG GCT T | 12485 | HUMNK65PRO | EPI-2206-01 | NFKB/NK & |
| | | | | TCell |
| | | | | Activating Prot |
| GGG TGG GCT TGG G | 12468 | HUMPEREEB | EPI 2206-02 | NFKB/Prostagl. |
| | | | | EP3 Rec |
| CCTGGGTGGGBBTGGG | 12469 | | EPI 2206-03 | HSNF2B/GCSF |
| | | | | NFKB/GranuLocCSF/ |
| | | | | Transcr. FactorNF2B |
| CCTGGBTGGGCBTGGG | 12470 | | EPI-2206-04 | HUMLAP/NFKB |
| | | | | Leuk.Adhes.Prot |
| GCCTGBGTGBBCTTGGG | 12471 | | EPI2206-05 | NFKB/Endothel |
| | | | | N2 S63833 |
| CCCAVGVCCVCCCAGGC | 11769 | | EPI 2206-06 | NFKBAS13/B Lymph |
| | | | | SerThrProt.Kinase |
| AGCCCACCCAGGC | 11770 | | EPI2206-07 | NFKBAS13/GCSF1 |
| | | | | HSGCSFR1Rec |
| BCCTGGGTGGGCTB | 11771 | | EPI2206-08 | NFKBAS13/GCSF1/ |
| | | | | NK7TCELLACT.Prot |
| GGTGGGCTTGGG | 11772 | | EPI 2206-09 | NFKBAS13/ |
| | | | | HSTGFB1 TGFB |
| CCBBGGTGGGCTTGGG | 11773 | | EPI 2206-10 | NFKBAS13/ |
| | | | | HSTGFB1 TGPB1 |
| CTGGGTGGGBBTGGG | 11774 | | EPI 2206-11 | NFKBAS13/ |
| | | | | HSGCSFR1 GCSFR1 |
| CCBGGGTGGGCTTGG | 11775 | | EPI 2206-12 | NFKBAS13/HUMCD30A |
| | | | | LymphActAntigCoding |
| GGGTGGGCTTGG | 11776 | | EPI-2206-12B | NFKBAS13/HUMCD30A |
| CCTGBGTGBGCBTGGG | 11777 | | EPI 2206-13 | NFKBAS13/HUMCAM1V |
| | | | | Vasc.Endoth.Cell |
| | | | | Adh.Molec |

B: Universal Base

The MTA oligos of Table 3 and others in accordance with this invention are suitable for use with two or more of the targets, such as those listed in Table 4 below.

TABLE 4
Targets for the MTA Oligos of Table 3

| Compound | Target |
|---|---|
| EPI 2010 | Adenosine A1 receptor |
| EPI 2045 | Adenosine A3 receptor |
| EPI 2873, EPI 2193 | NFκB |
| EPI 1873 | Interleukin-1 |
| EPI 1857 | Interleukin-5 |
| EPI 2945 | Interleukin-4 |
| EPI 2977 | Interleukin-8 |
| EPI 2031 | 5-Lipoxygenase |
| EPI 1898 | Leukotriene C-4 Synthase |
| EPI 1856 | Eotaxin |
| EPI 1131 | ICAM |
| EPI 1085 | VCAM |
| EPI 2085 | TNFα |
| EPI 1908 | PAF |
| EPI 1925 | IL-4 receptor |
| EPI 2643 | β2 aderenergic receptor kinase |
| EPI 2934 | Tryptase |
| EPI 2033 | Major Basic Protein |
| EPI 2795 | Eosinophil Peroxidase |

NfκB: nuclear factor κB
ICAM: intracellular adhesion molecule
VCAM: vascular cell adhesion molecule
TNF: tumor necrosis factor
PAF: platelet activating factor

The mRNA sequence of the targeted protein or the DNA sequence of the regulatory segment may be derived from the nucleotide sequence of the gene expressing or regulating the protein, whether for existing targets or those to be found in the future. Sequences for many target genes of different systems are presently known. See, GenBank data base, NIH, the entire sequences of which are incorporated here by reference. The sequences of those genes, whose sequences are not yet available, may be obtained by isolating the target segments applying technology known in the art. Once the sequence of the gene, its RNA and/or the protein are known, anti-sense oligonucleotides are produced as described above and utilized to validate the target by in vivo administration and testing for a reduction of the production of the targeted protein in accordance with standard techniques, and of specific functions. As already described above, the anti-sense oligonucleotides may be of any suitable length, e.g., from about 7 to about 60 nucleotides in length, depending on the particular target being bound and the mode of delivery thereof. The anti-sense oligonucleotide preferably is directed to an mRNA region containing a junction between intron and exon or to regions vicinal to the junction. Where the anti-sense oligonucleotide is directed to an intron/exon junction, it may either entirely overlie the junction or may be sufficiently close to the junction to inhibit splicing out of the intervening exon during processing of precursor mRNA to mature mRNA, e.g., with the 3′ or 5′ terminus of the anti-sense oligonucleotide being positioned within about, for example, 10, 5, 3, or 2 nucleotide of the intron/exon junction. Also preferred are anti-sense oligonucleotides which overlap the initiation codon and, more generally, those that target the coding region of the target mRNA. When practicing the present invention, the anti-sense oligonucleotide(s), administered, whether DNA or RNA may be related in origin to the species to which it is administered or to other species including prokaryotes. When treating humans, human anti-sense may be used if desired, except when targeting foreign invaders. Anti-sense oligos to endogenous sequences of other species, however, are also clearly encompassed.

Other agents that may be incorporated into the present composition are one or more of a variety of therapeutic agents which are administered to humans and animals. Some of the categories of agents suitable for incorporation into the present composition and formulations are analgesics, pre-menstrual medications, menopausal agents, anti-aging agents, anti-anxiolytic agents, mood disorder agents, anti-depressants, anti-bipolar mood agents, anti-schizophrenic agents, anti-cancer agents, alkaloids, blood pressure controlling agents, hormones, anti-inflammatory agents, muscle relaxants, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, analgesics, pre-menstrual medications, anti-menopausal agents such as hormones and the like, anti-aging agents, anti-anxiolytic agents, nociceptic agents, mood disorder agents, anti-depressants, anti-bipolar mood agents, anti-schizophrenic agents, anti-cancer agents, alkaloids, blood pressure controlling agents, hormones, anti-inflammatory agents, other agents suitable for the treatment and prophylaxis of diseases and conditions associated or accompanied with pain and inflammation, such as arthritis, burns, wounds, chronic bronchitis, chronic obstructive pulmonary disease (COPD), inflammatory bowel disease such as Crohn's disease and ulcerative colitis, autoimmune disease such as lupus erythematosus, muscle relaxants, steroids, soporific agents, anti-ischemic agents, anti-arrhythmic agents, contraceptives, vitamins, minerals, tranquilizers; neurotransmitter regulating agents, wound and burn healing agents, anti-angiogenic agents, cytokines, growth factors, anti-metastatic agents, antacids, anti-histaminic agents, anti-bacterial agents, anti-viral agents, anti-gas agents, agents for reperfusion injury, counteracting appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, etc.

Among the hormones suitable for active agents of the invention, are female and male sex hormones such as premarin, progesterone, androsterones and their analogues, thyroxine and glucocorticoids, including Budesonide, Dexamethasone, Flunisolide, Triamcinolone, and others. Among the libido altering agents are Viagra and other NO-level modulating agents, among the analgesics are over-the-counter medications such as ibuprofen, oruda, aleve and acetarinophen and controlled substances such as morphine and codeine, among the anti-depressants are tricyclics, MAO inhibitors and epinephrine, γ-amino butyric acid (GABA), dopamine and serotonin level elevating agents, e.g. Prozac, Amytryptilin, Wellbutrin and Zoloft among the skin renewal agents are Retin-A, hair growth agents such as Rogaine, among the anti-inflammatory agents are non-steroidal anti-inflammatory drugs (NSAIDs) and steroids, among the soporifics are melatonin and sleep inducing agents such as diazepam, cytoprotective, anti-ischemic and head injury agents such as enadoline, and many others. Examples of agents in the different groups are provided in the following list Examples of analgesics are Acetaminophen, Anilerdine, Aspirin, Buprenorphine, Butabital, Butoipphanol, Choline Salicylate, Codeine, Dezocine, Diclofenac, Diflunisal, Dihydrocodeine, Elcatoninin, Etodolac, Fenoprofen, Hydrocodone, Hydromorphone, Tbuprofen, Ketoprofen, Ketorolac, Levorphanol, Magnesium Salicylate, Meclofenamate, Mefenamic Acid, Meperidine, Methadone, Methotrireprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Examples of anti-anxiety agents include Alprazolari Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Examples of anti-anxiety agents associated with mental depression are Chlordiazepoxide, Amitriptyline, Loxapine Maprotiline and Perphenazine, among others. Examples of anti-inflammatory agents are non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicar, Salsalate, Sodium Salicylate, Sulindac, Tenoxicam, Tiaprofenic Acid, Tolmetin. Examples of anti-inflammatories for ocular treatment are Diclofenac, Flurbiprofen, Indomethacin, Ketorolac, Rimexolone (generally for post-operative treatment). Examples of anti-inflammatories for non-infectious nasal applications are Beclometaaxone, and the like. Examples of soporifics (anti-insomnia/sleep inducing agents) such as those utilized for treatment of insomnia, are Alprazolam, Bromazepam, Diazepam, Diphenhydramine, Doxylamine, Estazolar, Flurazepam, Halazepam, Ketazolam, Lorazepam, Nitrazepam, Prazepam Quazepam, Temazepam, Triazolam, Zolpidem and Sopiclone, among others. Examples of sedatives are Diphenhydramine, Hydroxyzine, Methotrimeprazine, Promethazine, Propofol, Melatonin, Trimeprazine, and the like. Examples of sedatives and agents used for treatment of petit mal and tremors, among other conditions, are Amitriptyline HCl, Chlordiazepoxide, Amobarbital, Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia) include Enadoline HCl (e.g. for treatment of severe head injury, orphan status, Warner Lambert). Examples of cytoprotective agents and agents for the treatment of menopause and menopausal symptoms are Ergotamine, Belladonna Alkaloids and Phenobarbitals. Examples of agents for the treatment of menopausal vasomotor symptoms are Clonidine, Conjugated Estrogens and Medroxyprogesterone, Estradiol, Estradiol Cypionate, Estradiol Valerate, Estrogens, conjugated Estrogens, esterified Estrone, Estropipate and Ethinyl Estradiol. Examples of agents for treatment of symptoms of Pre Menstrual Syndrome (PMS) are Progesterone, Progestin, Gonadotrophic Releasing Hormone, oral contraceptives, Danazol, Luprolide Acetate and Vitamnin B6. Examples of agents for the treatment of emotional/psychiatric treatments are Tricyclic Antidepressants including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications. Examples of anti-migraine agents are irnitrex and the like.

The amount of each active agent may be adjusted when, and if, additional agents with overlapping activities are included as discussed in this patent. The dosage of the active compounds, however, may vary depending on age, weight, and condition of the subject. Treatment may be initiated with a small dosage, e.g. less than the optimal dose, of the first active agent of the invention, whether an anti-inflammatory steroid or a ubiquinone, or both, and optionally other bioactive agents described above. This may be similarly done with the second active agent, until a desirable level is attained. Or vice versa, for example in the case of multivitamins and/or minerals, the subject may be stabilized at a desired level of these products and then administered the first active compound. The dose may be increased until a desired and/or optimal effect under the circumstances is reached. In general, the active agent is preferably administered at a concentration that will afford effective results without causing any unduly harmful or deleterious side effects, and may be administered either as a single unit dose, or if desired in convenient subunits administered at suitable times throughout the day. The second therapeutic or diagnostic agent(s) is (are) administered in amounts which are known in the art to be effective for the intended application. In cases where the second agent has an overlapping activity with the principal agent, the dose of one of the other or of both agents may be adjusted to attain a desirable effect without exceeding a dose range which avoids untoward side effects. Thus, for example, when other analgesic and anti-inflammatory agents are added to the composition, they may be added in amounts known in the art for their intended application or in doses somewhat lower that when administered by themselves.

Pharmaceutical compositions and kits comprising an anti-sense oligo and/or the non-corticoid steroid and/or ubiquinone including doses effective to reduce expression of target protein(s) by binding specifically with DNA or mRNA either encoding, or regulating the expression of the target proteins in the cell so as to prevent its translation are also part of the present invention. Such compositions are provided in a suitable pharmaceutically or veterinarily acceptable carrier(s), e.g., sterile pyrogen-free saline solution either separately or in combination when intended for dual administration, e.g. in a kit where both first and second agent are administered on specified dates whereas only one is administered other days. The active agents may be formulated with a hydrophobic carrier capable of passing through a cell membrane, e.g., in a liposome, with the liposomes carried in a pharmaceutically acceptable aqueous carrier. The oligonucleotides may also be coupled to a substance which inactivates mRNA, such as a ribozyme. Such oligonucleotides may be administered to a subject to inhibit the activation of a target, such as the adenosine receptors, which subject is in need of such treatment for any of the reasons discussed herein. Furthermore, the pharmaceutical formulation may also contain chimeric molecules comprising anti-sense oligonucleotides attached to molecules which are known to be internalized by cells. These oligonucleotide conjugates utilize cellular uptake pathways to increase cellular concentrations of oligonucleotides. Examples of macromolecules used in this manner include transferrin, asialoglycoprotein (bound to oligonucleotides via polylysine) and streptavidin. In the pharmaceutical formulation, the anti-sense compound may be contained within a lipid particle or vesicle, such as a liposome or microcrystal. The particles may be of any suitable structure, such as unilamellar or plurilamellar, so long as the anti-sense oligonucleotide is contained therein. Positively charged lipids such as N-[1-(2,3-dioleoyloxy)propyl]-N,N,N-trirethylammoniumethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

The active compounds provided in this patent are preferably administered to the subject as a pharmaceutical or veterinary composition. Pharmaceutical compositions for use in the present invention include formulations suitable for systemic and topical administration, including by inhalation, intrapuironary infusion, nasal, respirable, oral, topical (including buccal, sublingual, dermal and intraocular), parenteral (including subcutaneous, intradermal, intramuscular, intravenous and intraarticular), rectal, vaginal, ophthalnic, otical, implantable, and transdermal and iontophoretic administration, among others. The compositions may conveniently be provided in bulk, or presented in unit or multiple unit dosage form, and may be prepared by any of the methods well known in the art.

The first and second active compounds may be administered to the lungs, i.e. intrapulmonarily, nasally, respirably or by inhalation, of a subject by any suitable means. A preferred method of administration is by generating an aerosol or spray comprised of nasal or respirable particles comprising the active compound. The thus administered particles are then inhaled by the subject, i.e. by inhalation, intrapulmonary drip, or nasal administration, or by direct administration into the airways or respiration. The respirable particles may be liquid or solid, and they are preferably in the range of about 0.05, about 0.5, about 1, about 2, about 2.5 to about 3.5, about 4, about 6, about 8, about 10 micron, and preferably about 1 to about 5 micron (respirable or inhalable particles), or about 10, about 15, about 20, about 30 to about 50, about 100, about 150, about 200, about 300, about 400, about 500 micron, preferably about 10 to about 50, about 100 micron for intrapulmonary instillation or nasal administration. As explained above, particles of non-respirable size that are included in the aerosol or spray tend to deposit in the throat and be swallowed, and the quantity of non-respirable particles in the aerosol is preferably minimized. For nasal administration or intrapulmonary instillation, particularly for newborn babies and infants, a particle size in the range of about 10 to about 50 microns is preferred to ensure deposition and retention in the nasal or pulmonary cavity. Liquid pharmaceutical compositions of the active compound for producing an aerosol or spray may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. Another method would include passing through a mill and collecting the fine particles from the device for further classification. A solid particulate composition comprised of the active compound may optionally contain a dispersant that serves to facilitate the formation of an aerosol. A suitable dispersant is lactose, which may be blended with the active compound in any suitable ratio, e.g. a 1 to 2.5 ratio by weight. Again, other therapeutic and formulation compounds may also be included, such as a surfactant to improve the state of surfactant in the lung and help with the absorption of the active agent.

The dosage of the anti-sense compound administered will depend upon the disease being treated, the condition of the subject, the particular formulation, the route of administration, the timing of administration to a subject, etc. In general, intracellular concentrations of the oligonucleotide of from about 0.01, about 0.05, about 0.1, about 0.2, about 1 to about 5 μM, about 50 μM, about 100 μM or more, and more particularly about 0.2 to about 0.5 μM, are desired. For administration to a subject such as a human, a dosage of from about 0.01, about 0.1 or about 1 mg/Kg up to about 50, about 100, or about 150 mg/Kg and even higher doses are typically employed depending on the route of administration as is known in the art. Depending on the solubility of the particular formulation of active compound administered, the daily dose may be divided among one or several unit dose administrations. Administration of the anti-sense compounds may be carried out therapeutically (i.e., as a rescue treatment) or prophylactically. Aerosols of liquid particles comprising the active compound may be produced by any suitable means, such as with a nebulizer. See, e.g. U.S. Pat. No. 4,501,729. Nebulizers are commercially available devices that transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer comprise the active ingredient in a liquid carrier or diluent, the active ingredient comprising about 0.05 up to about 40% w/w of the composition, preferably about 1 to less than about 20% w/w. The carrier is typically water or a dilute aqueous alcoholic solution, preferably made isotonic with body fluids by the addition of, for example sodium chloride. Other carriers, however, are also suitable as an artisan would know. Optional additives include preservatives if the composition is not prepared sterile. An example of a preservative is methyl hydroxybenzoate, and other agents such as antioxidants, flavoring agents, volatile oils, buffering agents and surfactants, however, may also be added.

In one preferred embodiment, the pharmaceutical composition may further comprise one or more bronchodilating agents, and one or more surfactants along with a carrier and formulation agents alternatively, these active agents may beadministred separately. Suitable surfactants or surfactant components for enhancing the uptake of the anti-sense oligonucleotides of the invention include synthetic and natural as well as full and truncated forms of surfactant protein A, surfactant protein B, surfactant protein C, surfactant protein D and surfactant Protein E, partially and fully saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamellar bodies which are the natural carrier vehicles for the components of surfactant, omega-3 fatty acids, polyenic acid, polyenoic acid, lecithin, pallitinic acid, non-ionic block copolymers of ethylene or propylene oxides, polyoxypropylene, monomeric and polymeric, polyoxyethylene, monomeric and polymeric, poly (vinyl amine) with dextran and/or alkanoyl side chains, Brij 35, Triton X-100 and synthetic surfactants ALEC, Exosurf, Survan and Atovaquone, among others. These surfactants may be used either as a single, or as part of a multiple component, surfactant in a formulation, or as covalently bound additions to the 5′ and/or 3′ ends of the anti-sense oligo(s). Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or foil, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from about 0.1 to about 100 w/w of the formulation. A second type of illustrative aerosol generator comprises a metered dose inhaler. Metered dose inhalers are pressurized aerosol dispensers, typically containing a suspension or solution formulation of the active ingredient in a liquefied propellant. During the use these devices discharge the formulation through a valve adapted to deliver a metered volume, typically from about 10:1 to about 150:1, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents. The aerosol, whether formed from solid or liquid particles, may be produced by the aerosol generator for example at a rate of from about 10, about 30, about 70 to about 100, about 150, about 150 liters per minute, more preferably from about 30 to 150 liters per minute, and most preferably about 60 liters per minute. Aerosols containing greater amounts of medicament, however, may be administered more rapidly as is known in the art.

Aerosols of solid particles comprising the active compound may likewise be produced with any sold particulate medicament aerosol generator. Aerosol and spray generators for administering solid particulate medicaments to a subject, comprise product particles that are respirable or inhalable, and they generate a volume of aerosol containing a predetermined metered dose of a medicament at a rate suitable for human administration. Examples of such aerosol and spray generators include metered dose inhalers and insufflators known in the art. Liquid pharmaceutical compositions of active compound for producing an aerosol can be prepared by combining the anti-sense compound with the anti-inflammatory steroid(s) and/or the ubiquinone(s) and a suitable vehicle, such as sterile pyrogen free water. Other therapeutic compounds and formulation components may optionally be included as well. Solid particulate compositions containing respirable dry particles of micronized anti-sense compound may be prepared as known in the art, and generally described above, and then passing the micronized composition through a 400 mesh screen to break up or separate out large agglomerates.

Compositions suitable for oral administration may be presented in discrete units, such as capsules, cachets, lozenges, or tablets, each containing a pre-determined amount of the first and second active compounds; as a powder or granules; as a solution or a suspension in an aqueous or non-aqueous liquid; or as an oil-in-water or water-in-oil emulsion. Such compositions may be prepared by any suitable method of pharmacy that includes the step of bringing into association the active compounds and a suitable carrier. In general, the compositions of the invention are prepared by uniformly and intimately admixing the active compounds with a liquid or finely divided solid carrier, or both, and then, if necessary, shaping the resulting mixture. For example, tablet may be prepared by compressing or molding a powder or granules containing the active compound(s) alone, or optionally with one or more accessory ingredients. Compressed tablets may be prepared by compressing, in a suitable machine, the compound in a free-lowing form, such as a powder or granules optionally mixed with a binder, lubricant, inert diluent, and/or surface active/dispensing agent(s) or surfactants. Molded tablets way be made by molding, in a suitable machine, the powdered compound(s) moistened with an inert liquid binder. Compositions for oral administration may optionally include enteric coatings known in the art to prevent degradation of the compositions in the stomach and provide release of the drug in the small intestine.

Compositions suitable for buccal or sub-lingual administration include lozenges comprising the active compound in a flavored base, usually sucrose and acacia or tragacanth, and pastiles comprising the compound in an inert base such as gelatin and glycerin or sucrose and acacia.

Compositions suitable for parenteral administration comprise sterile aqueous and non-aqueous injection solutions, suspensions or emulsions of the active compound, which preparations are preferably isotonic with the blood of the intended recipient. These preparations may contain anti-oxidants, buffers, surfactants, bacteriostats, solutes which render the compositions isotonic with the blood of the intended recipient, and other formulation components known in the art. Aqueous and non-aqueous sterile suspensions may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions, suspensions and emulsions may be prepared from sterile powders, granules and tablets of the kind previously described.

Compositions suitable for topical application to the skin preferably take the form of an ointment, cream, lotion, paste, gel, spray, aerosol, or oil, although others are also suitable. Carriers that may be used include vaseline, lanoline, polyethylene glycols, alcohols, transdermal enhancers, and combinations of two or more thereof.

Compositions suitable for rectal and vaginal administration are also included and may be prepared by methods known in the art.

Compositions suitable for transdermal administration may be presented as discrete patches adapted to remain in intimate contact with the epidermis of the recipient for a prolonged period of time. Compositions suitable for transdermal administration may also be delivered by iontophoresis. See, e.g. Pharmaceutical Research 3:318 (1986). They typically take the form of an optionally buffered aqueous solution of the active compound containing appropriate ions to facilitate the iontophoretic delivery of the agent.

The relevant disclosures of all scientific publications and patent references cited in this patent are specifically intended to be incorporated herein by reference, particularly in reference to preparatory methods and technologies which are enabling of the invention. The following examples are provided to illustrate the present invention, and should not be construed as limiting thereon.

### EXAMPLES

In the following examples, μM means micromolar, ml means milliliters, μm means micrometers, mm means millimeters, cm means centimeters, EC means degrees Celsius, μg means micrograms, mg means milligrams, g means grams, kg means kilograms, M means molar, and h or hr. means hours.

### Example 1

### Design and Synthesis of Anti-Sense Oligonucleotides

The design of anti-sense oligonucleotides against the A₁ and A₃ adenosine receptors may require the solution of the complex secondary structure of the target A₁ receptor mRNA and the target A₃ receptor mRNA. After generating this structure, anti-sense nucleotide are designed which target regions of mRNA which might be construed to confer functional activity or stability to the mRNA and which optimally may overlap the initiation codon. Other target sites are readily usable. As a demonstration of specificity of the anti-sense effect, other oligonucleotides not totally complementary to the target mRNA, but containing identical nucleotide compositions on a w/w basis, are included as controls in anti-sense experiments.

The mRNA secondary structure of the adenosine A₁ receptor was analyzed and used as described above. to design a phosphorothioate anti-sense oligonucleotide. The anti-sense oligonucleotide which was synthesized was designated HAdA₁AS and had the following sequence: 5′-GAT GGA GGG CGG CAT GGC GGG-3′ (SEQ ID NO:9370). As a control, a mismatched phosphorothioate anti-sense nucleotide designated HAdAlMM1 was synthesized with the following sequence: 5′-GTA GCA GGC GGG GAT GGG GGC-3′ (SEQ ID NO:9371). Each oligonucleotide had identical base content and general sequence structure. Homology searches in GENBANK (release 85.0) and EMBL (release 40.0) indicated that the anti-sense oligonucleotide was specific for the human and rabbit adenosine A₁ receptor genes, and that the mismatched control was not a candidate for hybridization with any known gene sequence.

The secondary structure of the adenosine A₃ receptor mRNA was similarly analyzed and used as described above to design two phosphorothioate anti-sense oligonucleotides. The first anti-sense oligonucleotide (HAdA3AS1) synthesized had the following sequence: 5′-GTT GTT GGG CAT CTT GCC-3′ (SEQ ID NO:9372). As a control, a mismatched phosphorothioate anti-sense ohgonucleotide (HAdA3MM1) was synthesized, having the following sequence: 5′-GTA CTT GCG GAT CTA GGC-3′ (SEQ ID NO:9373). A second phosphorothioate anti-sense oligonucleotide (HAdA3AS2) was also designed and synthesized, having the following sequence: 5′-GTG GGC CTA GCT CTC GCC-3′ (SEQ ID NO:9374). Its control oligonucleotide (HAdA3MM2) had the sequence: 5′-GTC GGG GTA CCT GTC GGC-3′ (SEQ ID NO:9375). Phosphorothioate oligonucleotides were synthesized on an Applied Biosystems Model 396 Oligonucleotide Synthesizer, and purified using NENSORB chromatography (DuPont, Md.).

### Example 2

### In Vivo Testing of Adenosine A₁ Receptor Anti-sense Oligos

The anti-sense oligonucleotide against the human A₁ receptor (SEQ ID NO:9370) described above. was tested for efficacy in an in vitro model utilizing lung adenocarcinoma cells HTB-54. HTB-54 lung adenocarcinoma cells were demonstrated to express the A₁ adenosine receptor using standard northern blotting procedures and receptor probes designed and synthesized in the laboratory.

HTB-54 human lung adenocarcinoma cells (106/100 mm tissue culture dish) were exposed to 5.0:M HAdA1AS or HAdA1MM1 for 24 hours, with a fresh change of media and oligonucleotides after 12 hours of incubation. Following 24 hour exposure to the oligonucleotides, cells were harvested and their RNA extracted by standard procedures. A 21-mer probe corresponding to the region of mRNA targeted by the anti-sense (and therefore having the same sequence as the anti-sense, but not phosphorothioated) was synthesized and used to probe northern blots of RNA prepared from HAdA1AS-treated, HAdA1MM1-treated and non-treated HTB-54 cells. These blots showed clearly that HAdA1AS but not HAdA1MM1 effectively reduced human adenosine receptor mRNA by >50%. This result showed that HAdA1AS is a good candidate for an anti-asthma drug since it depletes intracellular mRNA for the adenosine A₁ receptor, which is involved in asthma.

### Example 3

### In Vivo Efficacy of Adenosine A₁ Receptor Anti-Sense Oligos

A fortuitous hoology between the rabbit and human DNA sequences within the adenosine A₁ gene overlapping the initiation codon permitted the use of the phosphorothioate anti-sense oligonucleotides initially designed for use against the human adenosine A₁ receptor in a rabbit model. Neonatal New Zealand white Pasteurella-free rabbits were immunized intraperitoneally within 24 hours of birth with 312 antigen units/ml house dust mite (D. farinae) extract (Berkeley Biologicals, Berkeley, Calif.), mixed with 10% kaolin. Immunizations were repeated weekly for the first month and then biweekly for the next 2 months. At 3-4 months of age, eight sensitized rabbits were anesthetized and relaxed with a mixture of ketamine hydrochloride (44 mg/kg) and acepromazine maleate (0.4 mg/kg) administered intramuscularly. The rabbits were then laid supine in a comfortable position on a small molded, padded animal board and intubated with a 4.0-mm intratracheal tube (Mallinkrodt, Inc., Glens Falls, N.Y.). A polyethylene catheter of external diameter 2.4 mm with an attached latex balloon was passed into the esophagus and maintained at the same distance (approximately 16 cm) from the mouth throughout the experiments. The intratracheal tube was attached to a heated Fleisch pneumotachograph (size 00; DOM Medical, Richmond, Va.), and flow was measured using a Validyne differential pressure transducer (Model DP-45161927; Validyne Engineering Corp., Northridge, Calif.) driven by a Gould carrier amplifier (Model 11-4113; Gould Electronic, Cleveland, Ohio). The esophageal balloon was attached to one side of the differential pressure transducer, and the outflow of the intratracheal tube was connected to the opposite side of the pressure transducer to allow recording of transpulmonary pressure. Flow was integrated to give a continuous tidal volume, and measurements of total lung resistance (RL) and dynamic compliance (Cdyn) were calculated at isovolumetric and flow zero points, respectively, using an automated respiratory analyzer (Model 6; Buxco, Sharon, Conn.). Animals were randomized and on Day 1 pretreatment values for PC₅₀ were obtained for aerosolized adenosine. Anti-sense (HAdA1AS) or mismatched control (HAdA1MM) oligonucleotides were dissolved in sterile physiological saline at a concentration of 5000 μg (5 mg) per 1.0 ml. Animals were subsequently administered the aerosolized anti-sense or mismatch oligonucleotide via the intratracheal tube (approximately 5000:g in a volume of 1.0 ml), twice daily for two days. Aerosols of either saline, adenosine, or anti-sense or mismatch oligonucleotides were generated by an ultrasonic nebulizer (DeVilbiss, Somerset, Pa.), producing aerosol droplets 80% of which were smaller than 5:m in diameter. In the first arm of the experiment, four randomly selected allergic rabbits were administered anti-sense oligonucleotide and four the mismatched control oligonucleotide. On the morning of the third day, PC₅₀ values (the concentration of aerosolized adenosine in mg/ml required to reduce the dynamic compliance of the bronchial airway 50% from the baseline value) were obtained and compared to PC₅₀ values obtained for these animals prior to exposure to oligonucleotide. Following a 1 week interval, animals were crossed over, with those previously administered mismatch control oligonucleotide now administered anti-sense oligonucleotide, and those previously treated with anti-sense oligonucleotide now administered mismatch control oligonucleotide. Treatment methods and measurements were identical to those employed in the first arm of the experiment. It should be noted that in six of the eight animals treated with anti-sense oligonucleotide, adenosine-mediated bronchoconstriction could not be obtained up to the limit of solubility of adenosine, 20 mg/ml. For the purpose of calculation, PC₅₀ values for these animals were set at 20 mg/ml. The values given therefore represent a minimum figure for anti-sense effectiveness. Actual effectiveness was higher. The results of this experiment are illustrated in Table 5 below.

TABLE 5
Effect of Adenosine A₁ Receptor Anti-sense Oligo
upon PC50 Values in Asthmatic Rabbits

| Mismatch Control | | A₁ Receptor Anti-sense Oligo | |
|---|---|---|---|
| Pre Oligo- | Post | Pre | Post |
| nucleotide | Oligonucleotide | Oligonucleotide | Oligonucleotide |
| 3.56 ± 1.02 | 5.16 ± 1.03 | 2.36 ± 0.68 | >19.5 ± 0.34** |

The results are presented as the mean (n = 8) ± SEM.
The significance was determined by repeated-measures analysis of variance (ANOVA), and Tukey's protected test.
**Significantly different from all other groups, p < 0.01.

In both arms of the experiment, animals receiving the anti-sense oligonucleotide showed an order of magnitude increase in the dose of aerosolized adenosine required to reduce dynamic compliance of the lung by 50%. No effect of the mismatched control oligonucleotide upon PC₅₀ values was observed. No toxicity was observed in any animal receiving either anti-sense or control inhaled oligonucleotide. These results show clearly that the lung has exceptional potential as a target for anti-sense oligonucleotide-based therapeutic intervention in lung disease. They further show, in a model system which closely resembles human asthma, that downregulation of the adenosine A₁ receptor largely eliminates adenosine-mediated bronchoconstriction in asthmatic airways. Bronchial hyperresponsiveness in the allergic rabbit model of human asthma is an excellent endpoint for anti-sense intervention since the tissues involved in this response lie near to the point of contact with aerosolized oligonucleotides, and the model closely simulates an important human disease.

### Example 4

### Specificity of A₁-Adenosine Receptor Anti-Sense Oligonucleotide

At the conclusion of the cross-over experiment of Example 3 above, airway smooth muscle from all rabbits was quantitatively analyzed for adenosine A₁ receptor number. As a control for the specificity of the anti-sense oligonucleotide, adenosine A₂ receptors, which should not have been affected, were also quantified. Airway smooth muscle tissue was dissected from each rabbit and a membrane fraction prepared according to the method of Kleinstein et al. (Kleinstein, J. and Glossrnann, H., Naunyn-Schrniedeberg's Arch Pharmacol. 305: 191-200 (1978)), the relevant portion of which is hereby incorporated in its entirety by reference, with slight modifications. Crude plasma membrane preparations were stored at −70 EC until the time of assay. Protein content was determined by the method of Bradford (M. Bradford, Anal. Biochem. 72, 240-254 (1976), the relevant portion of which is hereby incorporated in its entirety by reference). Frozen plasma membranes were thawed at room temperature and were incubated with 0.2 U/ml adenosine deaminase for 30 minutes at 37 EC to remove endogenous adenosine. The binding of [³H] DPCPX (A₁ receptor-specific) or [³H] CGS-21680 (A₁ receptor-specific) was measured as previously described by Ali et al. (Ali, S. et al., J. Pharmacol. Exp. Ther. 268, Ara. J. Physiol 266, L271-277 (1994), the relevant portion of which is hereby incorporated in its entirety by reference). The animals treated with adenosine A₁ anti-sense oligonucleotide in the cross-over experiment had a nearly 75% decrease in A₁ receptor number compared to controls, as assayed by specific binding of the A₁-specific antagonist DPCPX. There was no change in adenosine A₂ receptor number, as assayed by specific binding of the A₂ receptor-specific agonist 2-[p-(2-carboxyethyl)-phenethylamino]-5′-(N-ethylcarboxamido) adenosine (CGS-21680). This is illustrated in Table 6 below.

TABLE 6
Specificity of Action of Adenosine A₁
Receptor Oligonucleotide Anti-sense

| | Mismatch Control | A₁ Anti-sense |
|---|---|---|
| | Oligonucleotide | Oligonucleotide |
| A₁-Specific Binding | 1105 ± 48** | 293 ± 18 |
| A₂-Specific Binding | 302 ± 22 | 442 ± 171 |

The results are presented as the mean (n = 8) ± SEM.
The significance was determined by repeated-measures analysis of variance (ANOVA), and Tukey's protected test.
**Significantly different from mismatch control, p < 0.01.

The above results illustrate the effectiveness of anti-sense oligonucleotides in treating airway disease. Since the anti-sense oligos described above. eliminate the receptor systems responsible for adenosine-mediated bronchoconstiction, it may be less imperative to eliminate adenosine from them. However, it would be preferable to eliminate adenosine from even these oligonucleotides to reduce the dose needed to attain a similar effect. Described above are other anti-sense oligonucleotides targeting mRNA of proteins involved in inflammation. Adenosine has been eliminated from their nucleotide content to prevent its liberation during degradation.

### Example 5

### Anti-Sense Oligos Directed to Other Target Nucleic Acids

This work was conducted to demonstrate that the present invention is broadly applicable to anti-sense oligonucleotides (“oligos”) specific to nucleic acid targets broadly. The following experimental studies were conducted to show that the method of the invention is broadly suitable for use with anti-sense oligos designed as taught by this application and targeted to any and all adenosine receptor mRNAs. For this purpose, various anti-sense oligos were prepared to adenosine receptor mRNAs exemplified by the adenosine A₁, A_{2b} and A₃ receptor mRNAs. Anti-sense Oligo I was disclosed above (SEQ ID NO:9370). Five additional anti-sense phosphorothioate oligos were designed and synthesized as indicated above.

1-Oligo II (SEQ ID NO: 9376) also targeted to the adenosine A₁ receptor, but to a different region than Oligo I.

2-Oligo V (SEQ ID NO: 9379) targeted to the adenosine A_{2b} receptor.

3-Oligos III (SEQ ID NO: 9377) and IV (SEQ ID NO: 9378) targeted to different regions of the adenosine A₃ receptor.

4-Oligo I-PD (SEQ ED NO: 11050)(a phosphodiester oligo of the sane sequence as Oligo I).

These anti-sense oligos were designed for therapy on a selected species as described above and are generally specific for that species, unless the segment of the target mRNA of other species happens to contain a similar sequences. All anti-sense oligos were prepared as described below, and tested in vivo in a rabbit model for bronchoconstriction, inflammation and allergy, which have breathing difficulties and impeded lung airways, as is the case in ailments such as asthma, as described in the above-identified application.

### Example 6

### Design & Sequences of Other Anti-Sense Oligos

Six oligos and their effects in a rabbit model were studied and the results of these studies are reported and discussed below. Five of these oligos were selected for this study to complement the data on Oligo I (SEQ ID NO: 9370) provided in Examples 1 to 4 above. This oligo is anti-sense to one region of the adenosine A₁ receptor mRNA. The oligos tested are identified as anti-sense Oligos I (SEQ ID NO: 9370) and II (SEQ ID NO: 9376) targeted to a different region of the adenosine A₁ receptor mRNA, Oligo V (SEQ ID NO:9377) targeted to the adenosine A_{2b} receptor mRNA, and anti-sense Oligos III and IV (SEQ ID NOS: 9378 and 9379) targeted to two different regions of the adenosine A₃ receptor mRNA. The sixth oligo (Oligo I-PD) is a phosphodiester version of Oligo I (SEQ ID NO:9370). The design and synthesis of these anti-sense oligos was performed in accordance with Example 1 above.

(I) Anti-Sense Oligo I

The anti-sense oligonucleotide I referred to in Examples 1 to 4 above is targeted to the human A₁ adenosine receptor mRNA (EPI 2010). Anti-sense oligo I is 21 nucleotide long, overlaps the initiation codon, and has the following sequence:5′-GAT GGA GGG CGG CAT GGC GGG-3′(SEQ. ID NO:9370). The oligo I was previously shown to abrogate the adenosine-induced bronchoconstriction in allergic rabbits, and to reduce allergen-induced airway obstruction and bronchial hyperresponsiveness (BHR), as discussed above and shown by Nyce, J. W. & Metzger, W. J., Nature, 385:721 (1977), the relevant portions of which reference are incorporated in their entireties herein by reference.

(II) Anti-Sense Oligo H

A phosphorothioate anti-sense oligo (SEQ ID NO:9376) was designed in accordance with the invention to target the rabbit adenosine A₁ receptor mRNA region +936 to +956 relative to the initiation codon (start site). The anti-sense oligo II is 21 nucleotide long, and has the following sequence: 5′-CTC GTC GCC GTC GCC GGC GGG-3′ (SEQ ID NO:9376).

(III) Anti-Sense Oligo III

A phosphorothioate anti-sense oligo other than that provided in Example 1 above (SEQ ID NO:9377) was designed in accordance with the invention to target the anti-sense A₃ receptor mRNA region +3 to +22 relative to the initiation codon start site. The anti-sense oligo III is 20 nucleotide long, and has the following seqLuence: 5′-GGG TGG TGC TAT TGT CGG GC-3′ (SEQ ID NO:9377).

(IV) Anti-Sense Oligo IV

Yet another phosphorothioate anti-sense oligo (SEQ ID NO:9378) was designed in accordance with the invention to target the adenosine A₃ receptor mRNA region +386 to +401 relative to the initiation codon (start site). The anti-sense oligo IV is 15 nucleotide long, and has the following sequence: 5′-GGC CCA GGG CCA GCC-31 (SEQ ID NO:9378)

(V) Anti-Sense Oligo V

A phosphorothioate anti-sense oligo (SEQ ED NO:9379) was designed in accordance with the invention to arget the adenosine A_{2b} receptor mRNA region −21 to −1 relative to the initiation codon (start site). The anti-sense oligonucleotide V is 21 nucleotide long, and has the following sequence: 5′-GGC CGG GCC AGC CGG GCC CGG-3′ (SEQ ID NO:9379).

(VI) A₁ Mismnatch Oligos

Two different mismatched oligonucleotides having the following sequences were used as controls for anti-sense oligo I (SEQ ID NO: 1) described in Example 5 above: A₁ MM2:5′-GTA GGT GGC GGG CAA GGC GGG-3′ (SEQ ID NO:12490), and A₁ MM3:5′-GAT GGA GGC GGG CAT GGC GGG-3 (SEQ ID NO:12489). Anti-sense oligo I and the two misrnatch anti-sense oligos had identical base content and general sequence structure. Homology searches in GENBANK (release 85.0) and EMBL (release 40.0) indicated that the anti-sense oligo I was specific, not only for the human, but also for the rabbit, adenosine A₁ receptor genes, and that the mismatched controls were not candidates for hybridization with any known human or animal gene sequence.

(VII) Anti-Sense Oligo A₁-PD (Oligo VI)

A phosphodiester anti-sense oligo (Oligo VI; SEQ ID NO:9370) having the same nucleotide sequence as Oligo I was designed as disclosed in the above-identified application Anti-sense oligo I-PD is 21 nucleotide long, overlaps the initiation codon, and has the following sequence: 5′-GAT GGA GGG CGG CAT GGC GGG-3′ (SEQ ID NO:9370).

(III) Controls

Each rabbit was administered 5.0 ml aerosolized sterile saline following the same schedule as for the anti-sense oligos in (II), (III), and (IV) above.

### Example 7

### Synthesis of Anti-Sense Oligos

Phosphorothioate anti-sense oligos having the sequences described in (a) above, were synthesized on an Applied Biosystems Model 396 Oligonucleotide Synthesizer, and purified using NENSORB chromatography (DuPont, Del.). TETD (tetraethylthiuram disulfide) was used as the sulfirizing agent during the synthesis. Anti-sense oligonucleotide II (SEQ ID NO:9376), anti-sense oligonucleotide III (SEQ ID NO: 9377) and anti-sense oligonucleotide IV (SEQ ID NO: 9378) were each synthesized and purified in this manner.

### Example 8

### Preparation of Allergic Rabbits

Neonatal New Zealand white Pasturella-free rabbits were immunized intraperitoneally within 24 hours of birth with 0.5 ml of 312 antigen units/ml house dust mite (D. farinae) extract (Berkeley Biologicals, Berkeley, Calif.) mixed with 10% kaolin as previously described (Metzger, W. J., in Late Phase Allergic Reactions, Dorsch, W., Ed., CRC Handbook, pp. 347-362, CRC Press, Boca Raton (1990); Ali, S., Metzger, W. J. and Mustafa, S. I., Am J. Resp. Crit Care Med. 149: 908 (1994)), the relevant portions of which are incorporated in their entireties here by reference. Immunizations were repeated weekly for the first month and then biweekly until the age of 4 months. These rabbits preferentially produce allergen-specific IgE antibody, typically respond to aeroallergen challenge with both an early and late-phase asthmatic response, and show bronchial hyper responsiveness (BHR). Monthly intraperitoneal administration of allergen (312 units dust mite allergen, as above) continues to stimulate and maintain allergen-specific IgE antibody and BHR. At 4 months of age, sensitized rabbits were prepared for aerosol administration as described by Ali et al. (Ali, S., Metzger, W. J. and Mustafa, S. J., Am. J. Resp. Crit Care Med. 149 (1994)), the relevant section being incorporated in its entirety here by reference.

Dose-Response Studies

### Example 9

### Experimental Setup

Aerosols of either adenosine (0-20 mglnml), or anti-sense or one of two mismatch oligonucleotides (5 mg/ml) were separately prepared with an ultrasonic nebulizer (Model 646, DeVilbiss, Somerset, Pa.), which produced aerosol droplets, 80% of which were smaller than 5:m in diameter. Equal volumes of the aerosols were administered directly to the lungs via an intratracheal tube. The animals were randomized, and administered aerosolized adenosine. Day 1 pre-treatment values for sensitivity to adenosine were calculated as the dose of adenosine causing a 50% loss of compliance (PC₅₀ Adenosine). The animals were then administered either the aerosolized anti-sense or one of the mismatch anti-sense oligos via the intratracheal tube (5 mg/1.0 ml), for 2 minutes, twice daily for 2 days (total dose, 20 mg). Post-treatment PC₅₀ values were recorded (post-treatment challenge) on the morning of the third day. The results of these studies are provided in Example 21 below.

### Example 10

### Crossover Experiments

For some experiments utilizing anti-sense oligo I (SEQ ID NO: 9370) and a corresponding mismatch control oligonucleotide A₁MM2, following a 2 week interval, the animals were crossed over, with those previously administered the mismatch control A₁MM2, now receiving the anti-sense oligo I, and those previously treated with the anti-sense oligo I, now receiving the mismatch control A₁MM2 oligo. The number of animals per group was as follows. For mismatch A₁MM2 (Control 1), n=7, since one animal was lost in the second control arm of the experiment due to technical difficulties, for mismatch A₁MM3 n=4 (Control 2) and for A₁AS anti-sense oligo I, n=8. The A₁MM3 oligo-treated animals were analyzed separately and were not part of the cross-over experiment. The treatment methods and measurements employed following the cross-over were identical to those employed in the first arm of the experiment. In 6 of the 8 animals treated with the anti-sense oligo I (SEQ ID NO: 9370), no PC₅₀ value could be obtained for adenosine doses of up to 20 mg/ml, which is the limit of solubility of adenosine. Accordingly, the PC₅₀ values for these animals were assumed to be 20 mg/ml for calculation purposes. The values given, therefore, represent a minimum figure for the effectiveness of the anti-sense oligonucleotides of the invention. Other groups of allergic rabbits (n=4 for each group) were administered 0.5 or 0.05 mg doses of the anti-sense oligo I (SEQ ED NO: 9370), or the A₁MM2 oligo in the manner and according to the schedule described above (the total doses being 2.0 or 0.2 mg). The results of these studies are provided in Example 22 below.

### Example 11

### Anti-Sense Oligo Formulation

Each one of anti-sense oligos were separately solubilized in an aqueous solution and administered as described for anti-sense oligo I (SEQ ID NO:9370) in (e) above, in four 5 mg aliquots (20 mg total dose) by means of a nebulizer via endotracheal tube, as described above. The results obtained for anti-sense oligo I and its mismatch controls confirmed that the mismatch controls are equivalent to saline, as described in Example 19 below and in Table 1 of Nyce & Metzger, Nature 385: 721-725 (1997). Because of this finding, saline was used as a control for pulmonary function studies employing anti-sense oligos II, III and IV (SEQ ID NO: 9376, 9377 and 9378).

### Example 12

### Specificity of Oligo I for Adenosine A₁ Receptor (Receptor Binding Studies)

Tissue from airway smooth muscle was dissected to primary, secondary and tertiary bronchi from rabbits which had been administered 20 mg oligo I (SEQ ID NO: 9370) in 4 divided doses over a period of 48 hours as described above. A membrane fraction was prepared according to the method of Ali et al. (Ali, S., et al., Am. J. Resp. Crit Care Med. 149: 908 (1994), the relevant section relating to the preparation of the membrane fraction is incorporated in its entirety hereby by reference). The protein content was determined by the method of Bradford and plasma membranes were incubated with 0.2 U/ml adenosine deaminase for 30 minutes at 37 EC to remove endogenous adenosine. See, Bradford, M. M. Anal. Biochem. 72, 240-254 (1976), the relevant portion of which is hereby incorporated in its entirety by reference. The binding of [³H]DPCPX, [³H]NPC₁₇₇₃₁, or [³H]CGS-21680 was measured as described by Jarvis et al. See, Jarvis, M. F., et al., Pharmacol. Exptl. Ther. 251, 888-893 (1989), the relevant portion of which is fully incorporated herein by reference. The results of this study are shown in Table 8 and discussed in Example 20 below.

### Example 13

### Pulmonary Function Measurements (Compliance c_{DYN} and Resistance)

At 4 months of age, the immunized animals were anesthetized and relaxed with 1.5 ml of a mixture of ketamine HCl (35 mg/kg) and acepromazine maleate (1.5 mg/kg) administered intramuscularly. After induction of anesthesia, allergic rabbits were comfortably positioned supine on a soft molded animal board. Salve was applied to the eyes to prevent drying, and they were closed. The animals were then intubated with a 4.0 mm intermediate high-low cuffed Murphy 1 endotracheal tube (Mallinckrodt, Glen Falls, N.Y.), as previously described by Zavala and Rhodes. See, Zavala and Rhodes, Proc. Soc. Exp. Biol. Med. 144: 509-512 (1973), the relevant portion of which is incorporated herein by reference in its entirety. A polyethylene catheter of OD 2.4 mm (Becton Dickinson, Clay Adams, Parsippany N.J.) with an attached thin-walled latex balloon was passed into the esophagus and maintained at the same distance (approximately 16 cm) from the mouth throughout the experiment. The endotracheal tube was attached to a heated Fleisch pneumotach (size 00; DEM Medical, Richmond, Va.), and the flow (v) measured using a Validyne differential pressure transducer (Model DP-45-16-1927, Validyne Engineering, Northridge, Calif.), driven by a Gould carrier amplifier (Model 11-4113, Gould Electronics, Cleveland, Ohio). An esophageal balloon was attached to one side of the Validyne differential pressure transducer, and the other side was attached to the outflow of the endotracheal tube to obtain transpulmonary pressure (Pₜₚ). The flow was integrated to yield a continuous tidal volume, and the measurements of total lung resistance (Rₜ) and dynamic compliance (C_{dyn}) were made at isovolumetric and zero flow points. The flow, volume and pressure were recorded on an eight channel Gould 2000 W high-frequency recorder and C_{dyn} was calculated using the total volume and the difference in Pₜₚ at zero flow, and Rₜ was calculated as the ratio of Ptp and V at midtidal lung volumes. These calculations were made automatically with the Buxco automated pulnonary mechanics respiratory analyzer (Model 6, Buxco Electronics, Sharon, Conn.), as previously described by Giles et al. See, Giles et al., Arch. Int. Pharmacodyn. Ther. 194: 213-232 (1971), the relevant portion of which describing these calculations is incorporated in toto hereby by reference. The results obtained upon administration of oligo II on allergic rabbits are shown and discussed in Example 26 below.

### Example 14

### Measurement of Bronchial Hyperresponsiveness (BHR)

Each allergic rabbit was administered histamine by aerosol to determine their baseline hyperresponsiveness. Aerosols of either saline or hitamine were generated using a DeVilbiss nebulizer (DeVilbiss, Somerset, Pa.) for 30 seconds and then for 2 minutes at each dose employed. The ultrasonic nebulizer produced aerosol droplets of which 80% were <5 micron in diameter. The histamine aerosol was administered in increasing concentrations (0.156 to 80 mg/ml) and measurements of pulmonary function were made after each dose. The B4R was then determined by calculating the concentration of histamine (mg/ml) required to reduce the C_{dyn} 50% from baseline (PC_{50 Histamine})

### Example 15

### Cardiovascular Effect of Anti-Sense Oligo I

The measurement of cardiac output and other cardiovascular parameters using CardiomaxJ utilizes the principal of thermal dilution in which the change in temperature of the blood exiting the heart after a venous injection of a known volume of cool saline is monitored. A single rapid injection of cool saline was made into the right atrium via cannulation of the right jugular vein, and the corresponding changes in temperature of the mixed injectate and blood in the aortic arch were recorded via cannulation of the carotid artery by a temperature-sensing miniprobe. Twelve hours after the allergic rabbits had been treated with aerosols of oligo I (EPI 2010; SEQ ID NO: 9370) as described in (d) above, the animals were anesthetized with 0.3 ml/kg of 80% Ketamine and 20% Xylazine. This time point coincides with previous data showing efficacy for SEQ ID NO: 9370, as is clearly shown by Nyce & Metzger, (1997), supra, the pertinent disclosure being incorporated in its entirety here by reference. A thermocouple was then inserted into the left carotid artery of each rabbit, and was then advanced 6.5 cm and secured with a silk ligature. The right jugular vein was then cannulated and a length of polyethylene tubing was inserted and secured. A thermodilution curve was then established on a CardiomaxJ II (Columbus Instruments, Ohio) by injecting sterile saline at 20 EC to determine the correctness of positioning of the thermocouple probe. After establishing the correctness of the position of the thermocouple, the femoral artery and vein were isolated. The femoral vein was used as a portal for drug injections, and the femoral artery for blood pressure and heart rate measurements. Once constant baseline cardiovascular parameters were established, CardiomaxJ measurements of blood pressure, heart rate, cardiac output, total peripheral resistance, and cardiac contractility were made.

### Example 16

### Duration of Action of Oligo I (SEQ ID NO: 9370)

Eight allergic rabbits received initially increasing log doses of adenosine by means of a nebulizer via an intra-tracheal tube as described in (f) above, beginning with 0.156 mg/ml until compliance was reduced by 50% (PC_{50 Adenosine}) to establish a baseline. Six of the rabbits then received four 5 mg aerosolized doses of (SEQ ID NO: 9370) as described above. Two rabbits received equivalent amounts of saline vehicle as controls. Beginning 18 hours after the last treatment, the PC_{50 Adenosine} values were tested again. After this point, the measurements were continued for all animals each day, for up to 10 days. The results of this study are discussed in Example 25 below.

### Example 17

### Reduction of Adenosine A_{2b} Receptor Number by Anti-Sense Oligo V

Sprague Dawley rats were administered 2.0 mg respirable anti-sense oligo V (SEQ ID NO:9379) three times over two days using an inhalation chamber as described above. Twelve hours after the last administration, lung parenchymal tissue was dissected and assayed for adenosine A_{2b} receptor binding using [311]-NECA as described by Nyce & Metzger (1997), supra. Controls were conducted by administration of equal volumes of saline. The results are significant at p<0.05 using Student's paired t test, and are discussed in Example 28 below.

### Example 18

### Comparison of Oligo I & Corresponding Phosphodiester Oligo VI (SEQ ID NO:11050)

Oligo I (SEQ ID NO:9370) countered the effects of adenosine and eliminated sensitivity to it for adenosine amount up to 20 mg adenosine/5.0 ml (the limit of solubility of adenosine). Oligo VI (SEQ ID NO: 11050), the phosphodiester version of the oligonucleotide sequence, was completely ineffective when tested in the same manner. Both compounds have identical sequence, differing only in the presence of phosphorothioate residues in Oligo I (SEQ ID NO:9370), and were delivered as an aerosol as described above and in Nyce & Metzger (1997), supra. Significantly different at p<0.001, Student's paired t test. The results are discussed in Example 29 below.

Results Obtained for Anti-Sense Oligo I (SEQ ID NO: 1)

### Example 19

### Results of Prior Work

The nucleotide sequence and other data for anti-sense oligo I (SEQ D) NO: 9370), which is specific for the adenosine A₁ receptor, were provided above. The experimental data showing the effectiveness of oligo I in down regulating the receptor number and activity were also provided above. Further information on the characteristics and activities of anti-sense oligo I is provided in Nyce, J. W. and Metzger, W. J., Nature 385:721 (1997), the relevant parts of which relating to the following results are incorporated in their entireties herein by reference. The Nyce & Metzger (1997) publication provided data showing that the anti-sense oligo I (SEQ ID NO: 9370):
(1) The anti-sense oligo I reduces the number of adenosine A₁ receptors in the bronchial smooth muscle of allergic rabbits in a dose-dependent manner as may be seen in Table 5 below.
(2) Anti-sense Oligo I attenuates adenosine-induced bronchoconstriction and allergen-induced bronchoconstriction.
(3) The Oligo I attenuates bronchial hyperresponsiveness as measured by PC₅₀ histamine, a standard measurement to assess bronchial hyperresponsiveness. This result clearly demonstrates anti-inflammatory activity of the anti-sense oligo I as is shown in Table 5 above.
(4) As expected, because it was designed to target it, the anti-sense oligo I is totally specific for the adenosine A₁ receptor, and has no effect at all at any dose on either the very closely related adenosine A₂ receptor or the related bradykinin B₂ receptor. This is seen in Table 5 below.
(5) In contradistinction to the above effects of the Oligo I, the mismatch control molecules MM2 and MM3 (SEQ ID NO:11051 and SEQ ID NO:11052) which have identical base composition and molecular weight but differed from the anti-sense oligo I (SEQ ID NO: 9370) by 6 and 2 mismatches, respectively. These mismatches, which are the minimum possible while still retaining identical base composition, produced absolutely no effect upon any of the targeted receptors (A₁, A₂ or B₂).

These results, along with a complete lack of prior art on the use of anti-sense oligonucleotides, such as oligo I, targeted to the adenosine A₁ receptor, are unexpected results. The showings presented in this patent clearly enable and demonstrate the effectiveness, for their intended use, of the claimed agents and method for treating a disease or condition associated with lung airway, such as bronchoconstriction, inflammation, allergy(ies), and the like.

### Example 20

### Oligo I Significantly Reduces Response to Adenosine Challenge

The receptor binding experiment is described in Example 12 above, and the results shown in Table 5 below which shows the binding characteristics of the adenosine A₁-selective ligand [³H]DPCPX and the bradykinin B₂-selective ligand [³H]NPC 17731 in membranes isolated from airway smooth muscle of A₁ adenosine receptor and B₂ bradykinin receptor anti-sense- and mismatch-treated allergic rabbits.

TABLE 5
Binding Characteristics of Three Anti-Sense Oligos

| | |
|---|---|
| A₁ receptor | B₂ receptor |

| | | | | |
|---|---|---|---|---|
| Treatment¹ | Kd | Bₘₐₓ | Kd | Bmax |

Adenosine A₁ Receptor

| | | | | | |
|---|---|---|---|---|---|
| 20 | mg | 0.36 ± 0.029 nM | 19 ± 1.52 fmoles* | 0.39 ± 0.031 nM | 14.8 ± 0.99 fmoles |
| 2 | mg | 0.38 ± 0.030 nM | 32 ± 2.56 fmoles* | 0.41 ± 0.028 nM | 15.5 ± 1.08 fmoles |
| 0.2 | mg | 0.37 ± 0.030 nM | 49 ± 3.43 fmoles | 0.34 ± 0.024 nM | 15.0 ± 1.06 fmoles |

A₁MM1 (Control)

| | | | | | |
|---|---|---|---|---|---|
| 20 | mg | 0.34 ± 0.027 nM | 52.0 ± 3.64 fmoles | 0.35 ± 0.024 nM | 14.0 ± 1.0 fmoles |
| 2 | mg | 0.37 ± 0.033 nM | 51.8 ± 3.88 fmoles | 0.38 ± 0.028 nM | 14.6 ± 1.02 fmoles |

B₂A (Bradykinin Receptor)

| | | | | | |
|---|---|---|---|---|---|
| 20 | mg | 0.36 ± 0.028 nM | 45.0 ± 3.15 fmoles | 0.38 ± 0.027 nM | 8.7 ± 0.62 fmoles* |
| 2 | mg | 0.39 ± 0.035 nM | 44.3 ± 2.90 fmoles | 0.34 ± 0.024 nM | 11.9 ± 0.76 |
| 0.2 | mg | 0.40 ± 0.028 nM | 47.0 ± 3.76 fmoles | 0.35 ± 0.028 nM | 15.1 ± 1.05 fmoles |

B₂MM (Control)

| | | | | | |
|---|---|---|---|---|---|
| 20 | mg | 0.39 ± 0.031 nM | 42.0 ± 2.94 fmoles | 0.41 ± 0.029 nM | 14.0 ± 0.98 fmoles |
| 2 | mg | 0.41 ± 0.035 nM | 40.0 ± 3.20 fmoles | 0.37 ± 0.030 nM | 14.8 ± 0.99 fmoles |
| 0.2 | mg | 0.37 ± 0.029 nM | 43.0 ± 3.14 fmoles | 0.36 ± 0.025 nM | 15.1 ± 1.35 fmoles |

| | | | | |
|---|---|---|---|---|
| Saline Control | 0.37 ± 0.041 | 46.0 ± 5.21 | 0.39 ± 0.047 nM | 14.2 ± 1.35 fmoles |

### Example 21

### Dose-Response Effect of Oligo I

Anti-sense oligo I (SEQ ID NO:9370) was found to reduce the effect of adenosine administration to the animal in a dose-dependent manner over the dose range tested as shown in Table 6 below.

TABLE 6
Dose-Response Effect to Anti-sense Oligo I

| | |
|---|---|
| Total Dose | PC_{50 Adenosine} |
| (mg) | (mg Adenosine) |

Anti-sense Oligo I

| | |
|---|---|
| 0.2 | 8.32 ± 7.2 |
| 2.0 | 14.0 ± 7.2 |
| 20 | 19.5 ± 0.34 |

A₁MM2 oligo (control)

| | |
|---|---|
| 0.2 | 2.51 ± 0.46 |
| 2.0 | 3.13 ± 0.71 |
| 20 | 3.25 ± 0.34 |

The above results were studied with the Student's paired t test and found to be statistically different, p = 0.05

The oligo I (SEQ ID NO:9370), an anti-adenosine A₁ receptor oligo, acts specifically on the adenosine A₁ receptor, but not on the adenosine A₂ receptors. These results stem from the treatment of rabbits with anti-sense oligo I (SEQ ID NO: 9370) or mismatch control oligo (SEQ ID NO:11051; A₁MM2) as described in Example 9 above and in Nyce & Metzger (1997), supra (four doses of 5 mg spaced 8 to 12 hours apart via nebulizer via endotracheal tube), bronchial smooth muscle tissue excised and the number of adenosine A₁ and adenosine A₂ receptors determined as reported in Nyce & Metzger (1997), supra.

### Example 22

### Specificity of Oligo I (SEQ ID NO:9370) for Target Gene Product

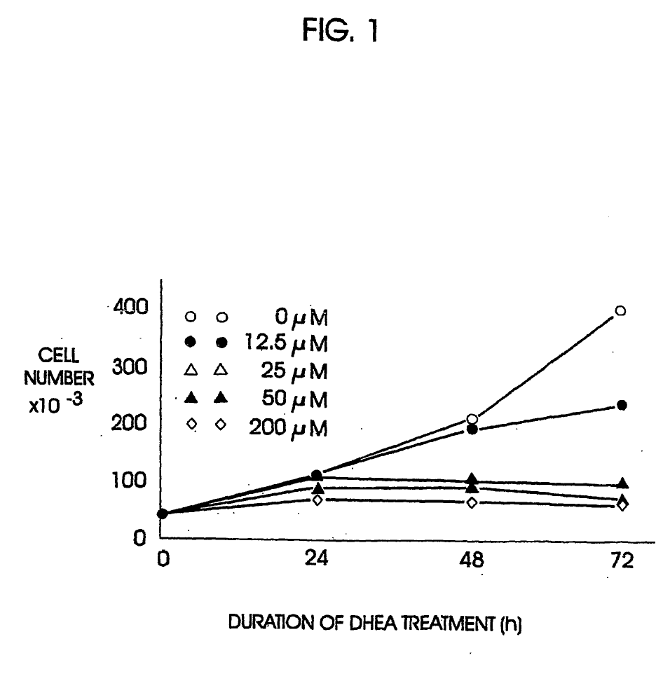

Oligo I (SEQ ID NO:9370) is specific for the adenosine A₁ receptor whereas its mismatch controls had no activity. FIG. 1 depicts the results obtained from the cross-over experiment described in Example 10 above and in Nyce & Metzger (1997), supra The two mismatch controls (SEQ ID NO:11051 and SEQ ID NO:11052) evidenced no effect on the PC_{50 Adenosine} value. On the contrary, the administration of anti-sense oligo I (SEQ ID NO:9370) showed a seven-fold increase in the PC_{50 Adenosine} value. The results clearly indicate that the anti-sense oligo I (SEQ ID NO: 9370) reduces the response (attenuates the sensitivity) to exogenously administered adenosine when compared with a saline control. The results provided in Table 6 above clearly establish that the effect of the anti-sense oligo I is dose dependent (see, column 3 of Table 5). The Oligo I was also shown to be totally specific for the adenosine A₁ receptor, (see, top 3 rows of Table), inducing no activity at either the closely related adenosine A₂ receptor or the bradykinin B₂ receptor (see, lines 8-10 of Table 6 above). In addition, the results shown in Table 6 establish that the anti-sense oligo I (SEQ ID NO:9370) decreases sensitivity to adenosine in a dose dependent manner, and that it does this in an anti-sense oligo-dependent manner since neither of two mismatch control oligonucleotides (A₁MM2; SEQ ID NO:11051 and A₁MM3; SEQ ED NO:11052) show any effect on PC_{50 Adenosine} alues or on attenuating the number of adenosine A₁ receptors.

### Example 23

### Effect on Aeroallergen-Induced Bronchoconstriction & Inflammation

The Oligo I (SEQ ID NO:9370) was shown to significantly reduce the histamine-induced effect in the rabbit model when compared to the mismatch oligos. The effect of the anti-sense Oligo I (SEQ ID NO:9370) and the mismatch oligos (A₁MM2, SEQ ID NO:11051 and A₁MM3, SEQ ID NO:11051) on allergen-induced airway obstruction and bronchial hyperresponsiveness was assessed in allergic rabbits. The effect of the anti-sense oligo I (SEQ ID NO:9370) on allergen-induced airway obstruction was assessed. As calculated from the area under the plotted curve, the anti-sense oligo I significantly inhibited allergen-induced airway obstruction when compared with the mismatched control (55%, p<0.05; repeated measures ANOVA, and Tukey's t test). A complete lack of effect was induced by the mismatch oligo A₁MM2 (Control) on allergen induced airway obstruction. The effect of the anti-sense oligo I (SEQ ID NO:9370) on allergen-induced BHR was determined as above. As calculated from the PC_{50 Histamine} value, the anti-sense oligo I (SEQ ID NO:9370) significantly inhibited allergen-induced 13HR in allergic rabbits when compared to the mismatched control (61%, p<0.05; repeated measures ANOVA, Tukey's t test). A complete lack of effect of the A₁MM mismatch control on allergen-induced BHR was observed. The results indicated that anti-sense oligo I (SEQ ID NO: 9370) is effective to protect against aeroallergen-induced bronchoconstriction (house dust mnite). In addition, the anti-sense oligo I (SEQ ID NO:9370) was also found to be a potent inhibitor of dust mite-induced bronchial hyper responsiveness, as shown by its effects upon histamine sensitivity which indicates anti-inflammatory activity for anti-sense oligo I (SEQ ID NO:1). The results indicated that anti-sense oligo I (SEQ ID NO 9370) is effective to protect against aeroallergen-induced bronchoconstriction (house dust mite). In addition, the anti-sense oligo I (SEQ ID NO: 9370) was also found to be a potent inhibitor of dust mite-induced bronchial hyper responsiveness, as shown by its effects upon histamine sensitivity which indicates anti-inflammatory activity for anti-sense oligo I (SEQ ID NO: 9370). The results indicated that anti-sense oligo I (SEQ ID NO: 9370) is effective to protect against aeroallergen-induced bronchoconstriction (house dust mite). In addition, the anti-sense oligo I (SEQ ID NO: 9370) was also found to be a potent inhibitor of dust mite-induced bronchial hyper responsiveness, as shown by its effects upon histamine sensitivity which indicates anti-inflammatory activity for anti-sense oligo I (SEQ ID NO: 9370). The results indicated that anti-sense oligo I (SEQ ID NO: 9370) is effective to protect against aeroallergen-induced bronchoconstriction (house dust mite). In addition, the anti-sense oligo I (SEQ ED NO: 9370) was also found to be a potent inhibitor of dust mite-induced bronchial hyper responsiveness, as shown by its effects upon histamine sensitivity which indicates anti-inflammatory activity for anti-sense oligo I (SEQ ID NO: 9370). The results indicated that anti-sense oligo I (SEQ ID NO: 9370) is effective to protect against aeroallergen-induced bronchoconstriction (house dust mite). In addition, the anti-sense oligo I (SEQ ID NO: 9370) was also found to be a potent inhibitor of dust mite-induced bronchial hyper responsiveness, as shown by its effects upon histamine sensitivity which indicates anti-inflammatory activity for anti-sense oligo I (SEQ ID NO: 9370). The results indicated that anti-sense oligo I (SEQ ID NO: 9370) is effective to protect against aeroallergen-induced bronchoconstriction (house dust mite). In addition, the anti-sense oligo I (SEQ ID NO: 9370) was also found to be a potent inhibitor of dust mite-induced bronchial hyper responsiveness, as shown by its effects upon histamine sensitivity which indicates anti-inflammatory activity for anti-sense oligo I (SEQ ID NO: 9370).

### Example 24

### Anti-sense Oligo I is Free of Deleterious Side Effects

The Oligo I (SEQ ID NO: 9370) was shown to be free of side effects that might be toxic to the recipient. No changes in arterial blood pressure, cardiac output, stroke volume, heart rate, total peripheral resistance or heart contractility (dPdT) were observed following administration of 2.0 or 20 mg oligo I (SEQ ID NO: 9370). The addition, the results of the measurement of cardiac output (CO), stroke volume (SV), mean arterial pressure (MAP), heart rate (HR), total peripheral resistance (TPR), and contractility (dPdT) with a CardiomaxJ apparatus (Columbus Instruments, Ohio) were assessed. These results evidenced that oligo I (SEQ ID NO: 9370) has no detrimental effect upon critical cardiovascular parameters. More particularly, this oligo does not cause hypotension. This finding is of particular importance because other phosphorothioate anti-sense oligonucleotides have been shown in the past to induce hypotension in some model systems. Furthermore, the adenosine A₁ receptor plays an important role in sinoatrial conduction within the heart. Attenuation of the adenosine A₁ receptor by anti-sense oligo I (SEQ ID NO: 9370) might be expected to result, therefore, in deleterious extrapulmonary activity in response to the downregulation of the receptor. This is not the case. The anti-sense oligo I (SEQ ID NO: 9370) does not produce any deleterious intrapulmonary effects and renders the administration of the low doses of the present anti-sense oligo free of unexpected, undesirable side effects. This demonstrates that when oligo I (SEQ ID NO: 9370) is administered directly to the lung, it does not reach the heart in significant quantities to cause deleterious effects. This is in contrast to traditional adenosine receptor antagonists like theophylline which do escape the lung and can cause deleterious, even life-threatening effects outside the lung.

### Example 25

### Long Lasting Effect of Oligo I

The Oligo I (SEQ ID NO: 9370) evidenced a long lasting effect as evidenced by the PC₅₀ and Resistance values obtained upon its administration prior to adenosine challenge. The duration of the effect was measured for with respect to the PC₅₀ of adenosine arnti-sense oligo I when administered in four equal doses of 5 mg each by means of a nebulizer via an endotracheal tube, as described above. The effect of the agent is significant over days 1 to 8 after administration. When the effect of the anti-sense oligo I (SEQ D NO: 9370) had disappeared, the animals were administered saline aerosols (controls), and the PC_{50 Adenosine} values for all animals were measured again. Saline-treated animals showed base line PC₅₀ adenosine values (n=6). The duration of the effect (with respect to Resistance) was measured for six allergic rabbits which were administered 20 mg of anti-sense oligo I (SEQ ID NO: 9370) as described above, upon airway resistance measured as also described above. The mean calculated duration of effect was 8.3 days for both PC₅₀ adenosine (p<0.05) and resistance (p<0.05). These results show that anti-sense oligo I (SEQ ID NO: 9370) has an extremely long duration of action, which is completely unexpected.

### Example 26

### Anti-Sense Oligo II

Anti-sense oligo II, targeted to a different region of the adenosine A₁ receptor mRNA, was found to be highly active against the adenosine A₁-mediated effects. The experiment measured the effect of the administration of anti-sense oligo II (SEQ ID NO: 9376) upon compliance and resistance values when 20 mg anti-sense oligo II or saline (control) were administered to two groups of allergic rabbits as described above. Compliance and resistance values were measured following an administration of adenosine or saline as described above in Example 13. The effect of the anti-sense oligo of the invention was different from the control in a statistically significant manner, p<0.05 using paired t-test, compliance; p<0.01 for resistance. The results showed that anti-sense oligo II (SEQ ID NO: 9376), which targets the adenosine A₁ receptor, effectively maintains compliance and reduces resistance upon adenosine challenge.

### Example 27

### Antisense Oligos III and IV

Oligos III (SEQ ID NO: 9377) and IV (SEQ ID NO: 9378) were shown to be in fact specifically targeted to the adenosine A₃ receptor by their effect on reducing inflammation and the number of inflammatory cells present upon separate administration of 20 mg of the anti-sense oligos III (SEQ ID NO: 9377) and IV (SEQ ID NO: 9378) to allergic rabbits as described above. The number of inflammatory cells was determined in their bronchial lavage fluid 3 hours later by counting at least 100 viable cells per lavage. The effect of anti-sense oligos III (SEQ ID NO: 9377) and IV (SEQ ID NO: 9378) upon granulocytes, and upon total cells in bronchial lavage were assessed following exposure to dust mite allergen. The results showed that the anti-sense oligo IV (SEQ ID NO: 9378) and anti-sense oligo III (SEQ ID NO: 9377) are very potent anti-inflammatory agents in the asthmatic lung following exposure to dust mite allergen. As is known in the art, granulocytes, especially eosinophils, are the primary inflammatory cells of asthma and the administration of anti-sense oligos III (SEQ ID NO: 9377) and IV (SEQ ID NO: 9378) reduced their numbers by 40% and 66%, respectively. Furthermore, anti-sense oligos IV (SEQ ID NO: 9378) and III (SEQ ID NO: 9376) also reduced the total number of cells in the bronchial lavage fluid by 40% and 80%, respectively. This is also an important indicator of anti-inflammatory activity by the present anti-adenosine A₃ agents of the invention. Inflammation is known to underlie bronchial hyperresponsiveness and allergen-induced bronchoconstriction in asthma. Both anti-sense oligonucleotides III (SEQ ID NO: 9377) and IV (SEQ ED NO: 9378), which are targeted to the adenosine A₃ receptor, are representative of an important new class of anti-inflammatory agents which may be designed to specifically target the lung receptors of each species.

### Example 28

### Anti-Sense Oligo V

The anti-sense oligo V (SEQ ID NO: 9379), targeted to the adenosine A_{2b} adenosine receptor mRNA was shown to be highly effective at countering adenosine A_{2b}-mediated effects and at reducing the number of adenosine A_{2b} receptors present to less than half.

### Example 29

### Unexpected Superiority of Substituted Over Phosphodiester-residue Oligo I-DS (SEQ ID NO:1681)

Oligos I (SEQ ID NO: 9370) and I-DS (SEQ ID NO: 11050) were separately administered to allergic rabbits as described above, and the rabbits were then challenged with adenosine. The phosphodiester oligo I-DS (SEQ ID NO: 11050) was statistically significantly less effective in countering the effect of adenosine whereas oligo I (SEQ ID NO: 9370) showed high effectiveness, evidencing a PC_{50 Adenosine} of 20 mg.

### Example 30

### Anti-Sense Oligo VI

For the present work, I designed an additional anti-sense phosphorothioate oligo targeted to the adenosine A₁ receptor (Oligo VI). This anti-sense oligo was designed for therapy on a selected species as described in the above patent application and is generally specific for that species, unless the segment of the adenosine receptor mRNA of other species elected happens to have a similar sequence. The anti-sense oligos were prepared as described below, and tested in vivo in a rabbit model for bronchoconstriction, inflammation and lung allergy, which have breathing difficulties and impeded lung airways, as is the case in ailments such as asthma, as described in the above-identified application. One additional oligo and its effect in a rabbit model was studied and the results of the study are reported and discussed below. The present oligo (anti-sense oligo VI) was selected for this study to complement the data on SEQ ID NO: 1 (Oligo I), which is anti-sense to the adenosine A₁ receptor mRNA provided in the above-identified patent application. This additional oligo is identified as anti-sense Oligo VI, and is targeted to a different region of the adenosine A₁ receptor mRNA than Oligo I. The design and synthesis of this anti-sense oligo was performed in accordance with the teaching, particularly Example 1, of the above-identified patent application. The anti-sense Oligo VI is a phosphorothioate designed to target the coding region of the rabbit adenosine A₁ receptor mRNA region +964 to +984 relative to the initiation codon (start site). The Oligo VI was prepared as described in the above-indicated application, and is 20 nucleotides long. The OligoVI is directed to the adenosine A₁ receptor gene, and has the following sequence: 5′-CGC CGG CGG GTG CGG GCC GG-3′ (SEQ ID NO: 12491). The phosphorothioate anti-sense Oligo VI having the sequence described in (5) above, was synthesized on an Applied Biosystems Model 396 Oligonucleotide Synthesizer, and purified using NENSORB chromatography (DuPont, Del.). TETD (tetraethylthiuram disulfide) was used as the sulfurizing agent during the synthesis.

### Example 31

### Preparation of Allergic Rabbits

Neonatal New Zealand white Pasturella-free rabbits were immunized intraperitoneally within 24 hours of birth with 0.5 ml of 312 antigen units/ml house dust mite (D. farinae) extract (Berkeley Biologicals, Berkeley, Calif.) mixed with 10% kaolin as previously described (Metzger, W. J., in Late Phase Allergic Reactions, Dorsch, W., Ed., CRC Handbook, pp 347-362, CRC Press, Boca Raton, 1990; AlH, S. Et al., Am J. Resp. Crit Care Med. 149: 908 (1994)). The immunizations were repeated weekly for the first month and then bi-weekly until the animals were 4 months old. These rabbits preferentially produce allergen-specific IgE antibody, typically respond to aeroallergen challenge with both an early and late-phase asthmatic response, and show bronchial hyper responsiveness (BHR). Monthly intraperitoneal administration of allergen (312 units dust mite allergen, as above) continues to stimulate and maintain allergen-specific IgE antibody and BHR. At 4 months of age, sensitized rabbits were prepared for aerosol administration as described by Ali et al. (1994), supra.

### Example 32

### Adenosine Aerosol Preparation

An adenosine aerosol (20 mg/ml) was prepared with an ultrasonic nebulizer (Model 646, DeVilbiss, Somerset, Pa.), which produced aerosol droplets, 80% of which were smaller than 5:m in diameter. Equal volumes of the aerosols were administered directly to the lungs via an intratracheal tube to all three rabbits. The animals were then administered the aerosolized adenosine and Day 1 pre-treatment values for sensitivity to adenosine were calculated as the dose of adenosine causing a 50% loss of compliance (PC₅₀ Adenosine). The animals were then administered the aerosolized anti-sense via the intratracheal tube (5 mg/1.0 ml), for 2 minutes, twice daily for 2 days (total dose, 20 mg). Post-treatment PC₅₀ values were recorded (post-treatment challenge) on the morning of the third day. The results of these studies are provided in (9) below.

### Example 33

### Anti-Sense Oligo Formulation

Each one of anti-sense oligos were separately solubilized in an aqueous solution and administered as described for anti-sense oligo I in (e) above, in four 5 mg aliquots (20 mg total dose) by means of a nebulizer via endotracheal tube, as described above.

### Example 34

### Oligo VI Reduces Response to Adenosine Challenge as Well or Better than Oligo I

Oligo VI was tested in three allergic rabbits of the characteristics and readied as described in (7) above and in the above-indicated patent application. Oligo VI targets a section of the coding region of the A₁ receptor which is different from Oligo I. Both these target sequences were selected randomly from many possible coding region target sequences. The three rabbits were treated identically as previously indicated for Oligo I. Briefly, 5 mg of Oligo VI were nebulized to the rabbits twice per day at 8 hour intervals, for two days. Thereafter, PC₅₀ adenosine studies were performed on the morning of the third day and compared to pre-treatment PC₅₀ values. This protocol is described in more detail in Nyce and Metzger (Nyce & Metzger, Nature 385: 721-725 (1997)). The results obtained for the three rabbits are shown in Table 7 below.

TABLE 7
PC₅₀ Adenosine before & after
Aerosolized Adenosine Treatment

| | PC₅₀ Adenosine |
|---|---|
| Treatment Time | (mg) |
| Pre-treatment | 3.0 ± 2.1 |
| Post-treatment | >20.0* |

*maximum achievable dose due to adenosine insolubility in saline

All three animals treated with Oligo VI completely eliminated sensitivity to adenosine up to the measurable level of the agent shown in Table 7 above. That is, the administration of the Oligo VI abrogated the adenosine-induced bronchoconstriction in the three allergic rabbits. The actual efficacy of Oligo VI is, therefore, greater than could be measured in the experimental system used. By comparing with the previously submitted results for the Oligo I, it may be seen that the Oligo VI was found to be as effective, or more, than Oligo I.

### Example 34

### Conclusions

The work described and results discussed in the examples clearly indicates that all anti-sense oligonucleotides designed in accordance with the teachings of the above-identified application were found to be highly effective at countering or reducing effects mediated by the receptors they are targeted to. That is, each and all of the two anti-sense oligos targeting an adenosine A₁ receptor mRNA, 1 anti-sense oligo targeting an adenosine A_{2b} receptor mRNA, and the 2 anti-sense oligos targeting an A₃ receptor mRNA were shown capable of countering the effect of exogenously administered adenosine which is mediated by the specific receptor they are targeted to. The activity of the anti-sense oligos of this invention, moreover, is specific to the target and substitutively fails to inhibit another target. In addition, the results presented also show that the administration of the present agents results in extremely low or non-existent deleterious side effects or toxicity. This represents 100% success in providing agents that are highly effective and specific in the treatment of bronchoconstriction and/or inflammation. This invention is broadly applicable in the same manner to all gene(s) and corresponding mRNAs encoding proteins involved in or associated with airway diseases. A comparison of the phosphodiester and a version of the same oligonucleotide wherein the phosphodiester bonds are substituted with phosphorothioate bonds evidenced an unexpected superiority for the phosphothiorate oligonucleotide over the phosphodiester anti-sense oligo.

### Example 35

### In Vivo Response to Adenosine Challenge with & without Oligo I Pretreatment

Two hyper responsive monkeys (ascaris sensitive) were challenged with inhaled adenosine, with and without pre-treatment with anti-sense oligo I (SEQ ID NO: 9370). The PC₄₀ adenosine was calculated from the data collected as being equivalent to that amount of adenosine in mg that causes a 40% decrease in dynamic compliance in hyper-responsive airways. The Oligo I (SEQ ID NO: 9370; EPI 2010) was subsequently administered at 10 mg/day for 2 days by inhalation. On the third day, the PC adenosine was again measured. The PC₄₀ adenosine value prior to treatment with Oligo I was compared side-by-side with to the PC₄₀ adenosine taken after administration of Oligo I (Figure not shown). The results of the experiment conducted with two animals showed that any sensitivity to adenosine was completely eliminated by the administration of the oligo of this invention in one animal, and substantially reduced in the second.

### Example 36

### Extension of the Experimental Results

The method of the present invention is also practiced with anti-sense oligonucleotides targeted to many genes, mRNAs and their corresponding proteins as described above, in essentially the same manner as given above, for the treatment of various conditions in the lungs. Examples of these are Human A₂ₐ adenosine receptor, Human A_{2b} adenosine receptor, Human IgE receptor β, Human Fc-epsilon receptor CD23 antigen (IgE receptor), Human IgE receptor, α subunit, Human IgE receptor, Fc epsilon R, Human histidine decarboxylase, Human beta tryptase, Human tryptase-I, Human prostaglandin D synhase, Human cyclooxygenase-2, Human eosinophil cationic protein, Human eosinophil derived neurotoxin, Human eosinophil peroxidase, Human intercellular adhesion molecule-1 (ICAM-1), Human vascular cell adhesion molecule 1 (VCAM-1), Human endothelial leukocyte adhesion molecule (ELAM-1), Human P Selectin, Human endothelial monocyte activating factor, Human IL3, Human IL4, Human IL5, Human IL6, Human monocyte-derived neutrophil chemotactic factor, Human neutrophil elastase (medullasin), Human neutrophil oxidase factor, Human cathepsin G, Human defensin 1, Human defensin 3, Human macrophage inflammatory protein-1-alpha, Human muscarinic acetylcholine receptor HM1, Human muscarinic acetylcholine receptor HM3, Human fibronectin, Human interleukin 8, Human GM-CSF, Human tumor necrosis factor α, Human leukotriene C₄ synthase, Human major basic protein, and many more.

### Example 37

### In Vivo Effects of Folinic Acid and DHEA on Adenosine Levels

In the examples provided below, EA means an epiandrosterone, DHEA means dehydroepiandrosterone, s means seconds, mg means milligrams, kg means kilograms, kw means kilowatts, Mhz means megahertz, CoQ means a ubiquinone, and nmol means nanomoles.

Young adult male Fischer 344 rats (120 grams) were administered dehydroepiandrosterone (DHEA) (300 mg/kg) or methyltestosterone (40 mg/kg) in carboxymethylceuulose by gavage once daily for fourteen days. Folinic acid (50 mg/kg) was administered intraperitoneally once daily for fourteen days. On the fifteenth day, the animals were sacrificed by microwave pulse (1.33 kw, 2450 MHZ, 6.5 s) to the cranium, which instantly denatures all brain protein and prevents further metabolism of adenosine. Hearts were removed from animals and flash frozen in liquid nitrogen with 10 seconds of death. Liver and lungs were removed en bloc and flash frozen with 30 seconds of death. Brain tissue was subsequently dissected. Tissue adenosine was extracted, derivatized to 1, N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 1 below. Results are expressed as the mean±SEM, with ? p<0.05 compared to control group and ψ p<0.05 compared to DHEA or methyltestosterone-treated groups.

TABLE 1
In Vivo Effect of DHEA, δ-1-methyltestosterone &
Folinic Acid on Adenosine Levels in Various Rat Tissues

| | Intracellular Adenosine | | |
|---|---|---|---|
| | (nmol/mg protein) | | |
| | Heart | Lung | Brain |
| Control | 10.6 ± 0.6 | 3.1 ± 0. | 0.5 ± 0.04 |
| | (n = 12) | (n = 6) | (n = 12) |
| DHEA | 6.7 ± 0.5 | 2.3 ± 0.3 | 0.19 ± 0.01 |
| (300 mg/kg) | (n = 12) | (n = 6) | (n = 12) |
| Methyltestosterone | 8.3 ± 1.0 | N.D. | 0.42 ± 0.06 |
| (40 mg/kg) | (n = 6) | | (n = 6) |
| Methyltestost. (M) | 6.0 ± 0.4 | N.D. | 0.32 ± 0.03 |
| (120 mg/kg) | (n = 6) | | (n = 6) |
| Folinic Acid (F.A.) | 12.4 ± 2.1 | N.D. | 0.72 ± 0.09 |
| (50 mg/kg) | (n = 5) | | (n = 5) |
| DHEA + F.A. | 11.1 ± 0.6 | N.D. | 0.55 ± 0.09 |
| (300 mg/kg; 50 mg/kg) | (n = 5) | | (n = 5) |
| M + F.A. | 9.1 ± 0.4 | N.D. | 0.60 ± 0.06 |
| (120 mg/kg; 50 mg/kg) | (n = 6) | | (n = 6) |

N.D. = Not Determined

The results of these experiments indicate that rats administered DHEA or methyltestosterone daily for two weeks showed multi-organ depletion of adenosine. Depletion was dramatic in brain (60% depletion for DHEA, 34% for high dose methyltestosterone) and heart (37% depletion for DHEA, 22% depletion for high dose methyltestosterone). Co-administration of folinic acid completely abrogated steroid-mediated adenosine depletion. Folinic acid administered alone induce increase in adenosine levels for all organs studied.

### Example 38

### Preparation of the Experimental Model

Cell cultures, HT-29 SF cells, which represent a subline of HY-29 cells (ATCC, Rockville, Md.) and are adapted for growth in completely defined serum-free PC-1 medium (Ventrex, Portland, Me.), were obtained. Stock cultures were maintained in this medium at 37° in a humidified atmosphere containing 5% CO₂. At confluence cultures were replated after dissociation using trypsin/EDTA (Gibco, Grand Island, N.Y.) and re-fed every 24 hours. Under these conditions, the doubling time for HT-29 SF cells during logarithmic growth was 24 hours.

### Example 39

### Flow Cytometry

Cells were plated at 10⁵/60-rm dish in duplicate. For analysis of cell cycle distribution, cultures were exposed to either 0, 25, 50, or 200 μM DHEA. For analysis of reversal of cell cycle effects of DHEA, cultures were exposed to either 0 or 25 μM DHEA, and the media were supplemented with MVA, CH, RN, MVA plus CH, or MVA plus CH plus RN or were not supplemented. Cultures were trypsinized following 0, 24, 48, or 74 hours and fixed and stained using a modification of a procedure of Bauer et al., Cancer Res., 46, 3173-3178 (1986). Briefly, cells were collected by centrifugation and resuspended in cold phosphate-buffered saline. Cells were fixed in 70% ethanol, washed, and resuspended in phosphate-buffered saline. One ml hypotonic stain solution [50 μg/ml propidium iodide (Sigma Chemical Co.), 20 μg/ml Rnase A (Boehringer Mannheim, Indianapolis, Ind.), 30 mg/ml polyethylene glycol, 0.1% Triton X-100 in 5 mM citrate buffer] was then added, and after 10 min at room temperature, 1 ml of isotonic stain solution (propidium iodide, polyethylene glycol, Triton X-100 in 0.4M NaCl] was added and the cells were analyzed using a flow cytometer, equipped with pulse width/pulse area doublet discrimination (Becton Dickinson Immunocytometry Systems, San Jose, Calif.) After calibration with fluorescent beads, a minimum of 2×10⁴ cells/sample were analyzed, data were displayed s total number of cells in each of 1024 channels of increasing fluorescence intensity, and the resulting histogram was analyzed using the Cellfit analysis program (Becton Dickinson).

### Example 40

### DHEA Effect on Cell Growth

Cells were plated 25,000 cells/30 mm dish in quadruplicate, and after 2 days received 0, 12.5, 25, 50, or 200 μM DHEA. Cell number was determined 0, 24, 48, and 72 hours later using a Coulter counter (model Z; Coulter Electronics, Inc. Hialeah, Fla.). DHEA (AKZO, Basel, Switzerland) was dissolved in dimethyl sulfoxide, filter sterilized, and stored at −20° C. until use.

FIG. 1 illustrates the inhibition of growth for HT-29 cells by DHEA. Points refer to numbers of cells, and bars refer to SEM. Each data point was performed in quadruplicate, and the experiment was repeated three times. Where SEM bars are not apparent, SEM was smaller than symbol. Exposure to DHEA resulted in a reduced cell number compared to controls after 72 hours in 12.5 μM, 48 hours in 25 or 50 μM, and 24 hours in 200 μM DHEA, indicating that DHEA produced a time- and dose-dependent inhibition of growth.

### Example 41

### DHEA Effect on Cell Cycle

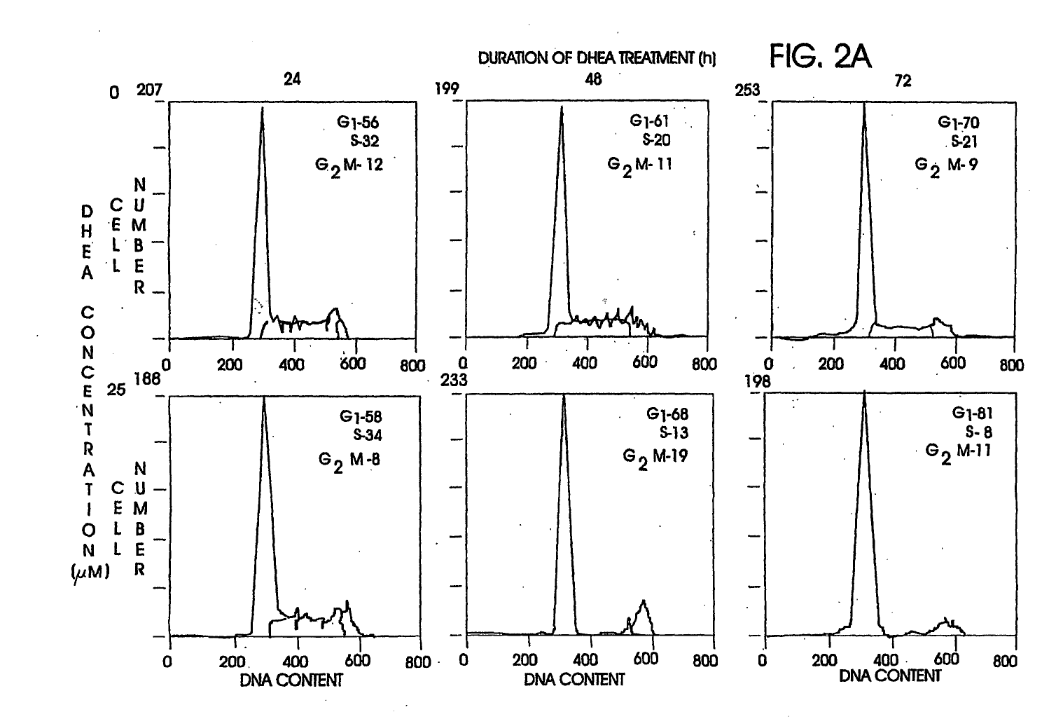

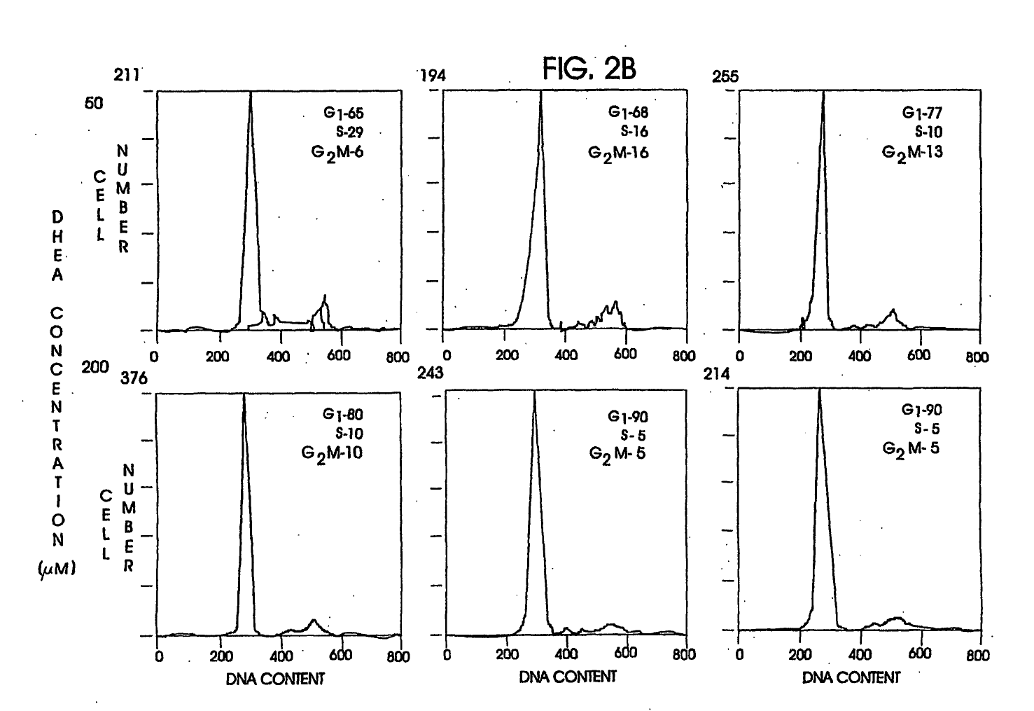

To examine the effects of DHEA on cell cycle distribution, HT-29 SF cells were plated (10⁵ cells/60 mm dish), and 48 hours later treated with 0, 25, 50, or 200 μM DHEA. FIG. 2 illustrates the effects of DHEA on cell cycle distribution in HT-29 SF cells. After 24, 48, and 72 hours, cells were harvested, fixed in ethanol, and stained with propidium iodide, and the DNA content/cell was determined by flow cytometric analysis. The percentage of cells in G₁, S, and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased, and the percentage of cells in G₁, S and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased and the percentage of cells in G₁ phase was increased after 72 hours. A transient increase in G₂M phase cells was apparent after 48 hours. Exposure to 200 μM DHEA produced a similar but more rapid increase in the percentage of cells in G₁ and a decreased proportion of cells in S phase after 24 hours, which continued through the treatment. This indicates that DHEA produced a G₁ block in HT-29 SF cells in a time-and dose-dependent manner.

### Example 42

### Reversal of DHEA-Mediated Effect on Growth & Cell Cycle

Reversal of DHEA-mediated Growth Inhibition. Cells were plated as above, and after 2 days received either 0 or 25 μM DHEA-containing medium supplemented with mevalonic acid (“MVA”; 2 mM) squalene (“SQ”; 80 μM), cholesterol (“CH”; 15 μg/ml), MVA plus CH, ribonucleosides (“RN”; uridine, cytidine, adenosine, and guanosine at final concentrations of 30 μM each), deoxyribonucleosides (“DN”; thymidine, deoxycytidine, deoxyadenosine and deoxyguanosine at final concentrations of 20 μM each). RN plus DN, or MVA plus CH plus RN, or medium that was not supplemented. All compounds were obtained from Sigma Chemical Co. (St Louis, Mo.) Cholesterol was solubilized in ethanol immediately before use. RN and DN were used in maximal concentrations shown to have no effects on growth in the absence of DHEA.

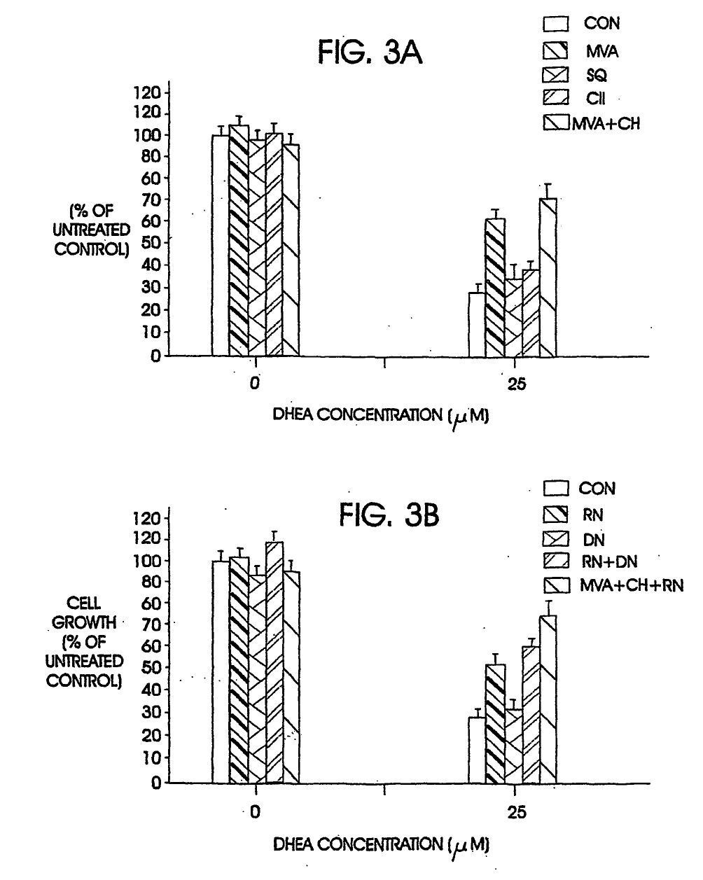

FIG. 3 illustrates the reversal of DHEA-induced growth inhibition in HT-29 SF cells. In A, the medium was supplemented with 2 μM MVA, 80 μM SQ, 15 μg/ml CH, or MVA plus CH (MVA+CH) or was not supplemented (CON). In B, the medium was supplemented with a mixture of RN containing uridine, cytidine, adenosine, and guanosine in final concentrations of 30 μM each; a mixture of DN containing thymidine, deoxycytidine, deoxyadenosine and deoxyguanosine in final concentrations of 20 μM each; RN plus DN (RN+DN); or MVA plus CH plus RN (MVA+CH+RN). Cell numbers were assessed before and after 48 hours of treatment, and culture growth was calculated as the increase in cell number during the 48 hour treatment period. Columns represent cell growth percentage of untreated controls; bars represent SEM. Increase in cell number in untreated controls was 173,370±6518. Each data point represents quadruplicate dishes from four independent experiments. Statistical analysis was performed using Student's t test; ψ p<0.01; κ p<0.001; compared to treated controls. Note that supplements had little effect on culture growth in absence of DHEA.

Under these conditions, the DHEA-induced growth inhibition was partially overcome by addition of MVA as well as by addition of MVA plus CH. Addition of SQ or CH alone had no such effect. This suggest that the cytostatic activity of DHEA was in part mediated by depletion of endogenous mevalonate and subsequent inhibition of the biosynthesis of an early intermediate in the cholesterol pathway that is essential for cell growth. Furthermore, partial reconstitution of growth was found after addition of RN as well as after addition of RN plus DN but not after addition of DN, indicating that depletion of both mevalonate and nucleotide pools is involved in the growth-inhibitory action of DHEA. However, none of the reconstitution conditions including the combined addition of MVA, CH, and RN completely overcame the inhibitory action of DHEA, suggesting either cytotoxic effects or possibly that additional biochemical pathways are involved.

### Example 43

### Reversal of DHEA Effect on Cell Cycle

HT-29 SF cells were treated with 25 FM DHEA in combination with a number of compounds, including MVA, CH, or RN, to test their ability to prevent the cell cycle-specific effects of DHEA. Cell cycle distribution was determined after 48 and 72 hours using flow cytometry.

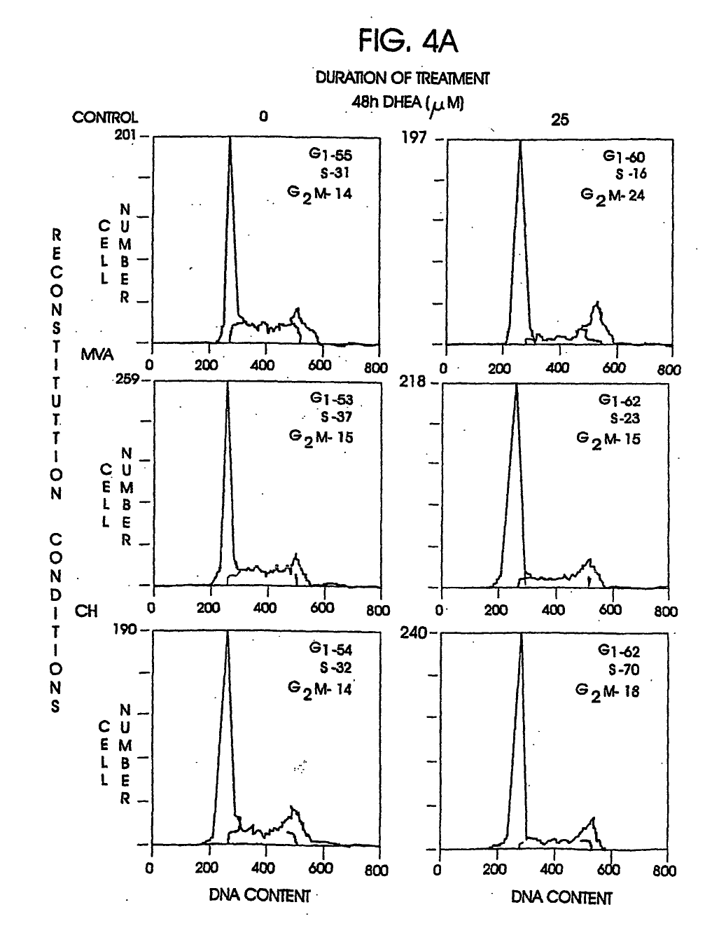

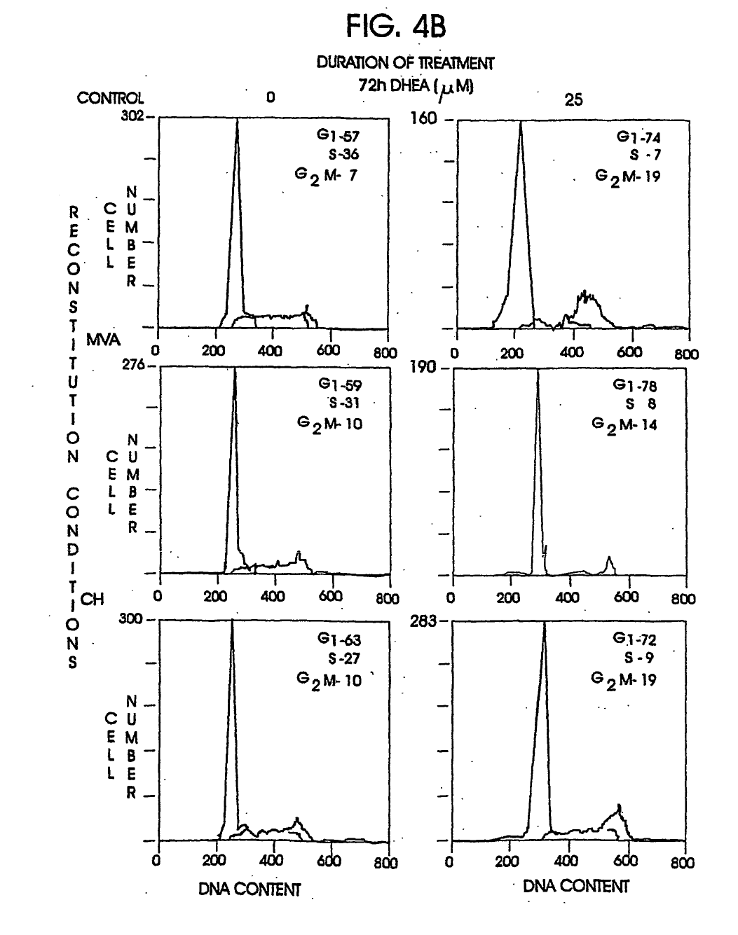

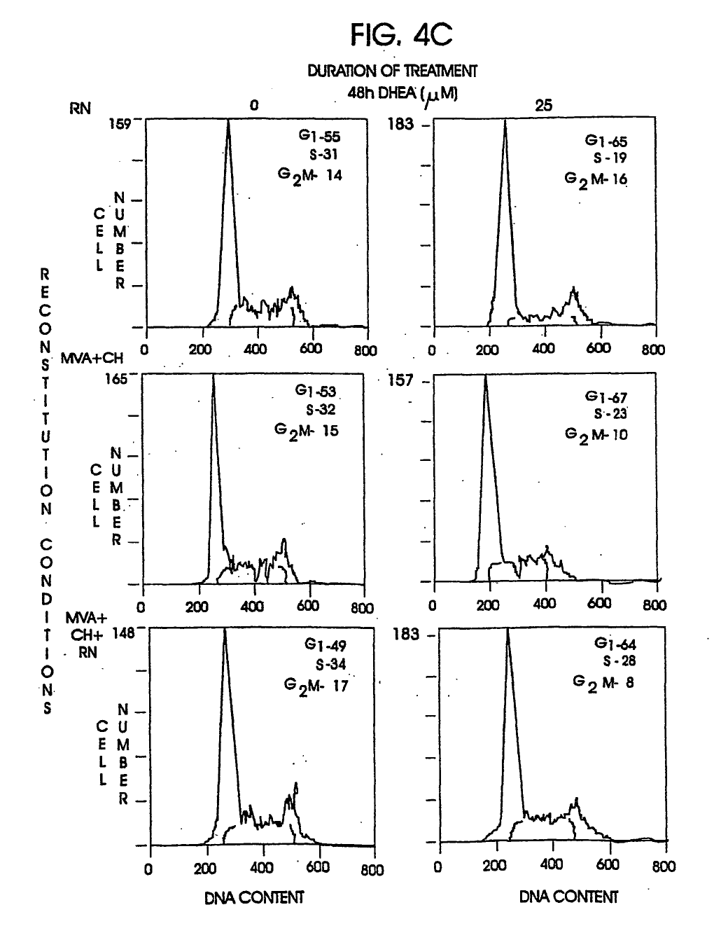

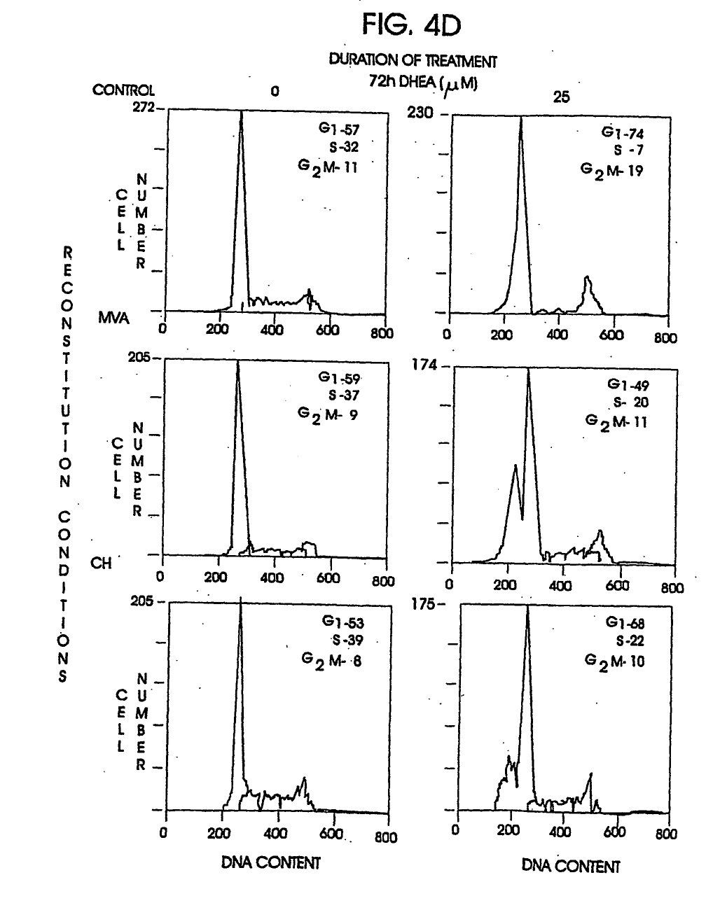

FIG. 4 illustrates reversal of DHEA-induced arrest in HT-29 SF cells. Cells were plated (10⁵ cells/60 mm dish) and 48 hours later treated with either 0 or 25 FM DHEA. The medium was supplemented with 2 FM MVA; 15 Fg/ml CH; a mixture of RN containing uridine, cytidine, adenosine, and guanosine in final concentrations of 30 FM; MVA plus CH (MVA+CH); or MVA plus CH plus RN (MVA+CH+RN) or was not supplemented. Cells were harvested after 48 or 72 hours, fixed in ethanol, and stained with propidium iodine, and the DNA content per cell was determined by flow cytometric analysis. The percentage of cells in G₁, S, and G₂M phases were calculated using the Cellfit cell cycle profile analysis program. S phase is marked by a quadrangle for clarity. Representative histograms from duplicative determinations are shown. The experiment was repeated two times. Note that supplements had little effect on cell cycle progression in the absence of DHEA.

With increasing exposure time, DHEA progressively reduced the proportion of cells in S phase. While inclusion of MVA partially prevented this effect in the initial 48 hours but not after 72 hours, the addition of MVA plus CH was also able to partially prevent S phase depletion at 72 hours, suggesting a requirement of both MVA and CH for cell progression during prolonged exposure. The addition of MVA, CH, and RN was apparently most effective at reconstitution but still did not restore the percentage of S phase cells to the value seen in untreated control cultures. CH or RN alone had very little effect at 48 hours and no effect at 72 hours. Morphologically, cells responded to DHEA by acquiring a rounded shape, which was prevented only by the addition of MVA to the culture medium (data not shown). Some of the DNA histograms after 72 hours DHEA exposure in FIG. 4 also show the presence of a subpopulation of cells possessing apparently reduced DNA content. Since the HT-29 cell line is known to carry populations of cells containing varying numbers of chromosomes (68-72; ATCC), this may represent a subset of cells that have segregated carrying fewer chromosomes.

### Example 44

### Conclusions

The examples above provide evidence that in vitro exposure of HT-29 SF human colonic adenocarcinoma cells to concentrations of DHEA known to deplete endogenous mevalonate results in growth inhibition and G₁ arrest and that addition of MVA to the culture medium in part prevents these effects. DHEA produced effects upon protein isoprenylation which were in many respects similar to those observed for specific 3-hydroxy-3-methyl-glutaryl-CoA reductase inhibitors such as lovastatin and compactin. Unlike direct inhibitors of mevalonate biosynthesis, however, DHEA mediates its effects upon cell cycle progression and cell growth in a pleiotropic manner involving ribo-and deoxyribonucleotide biosynthesis and possibly other factors as well. The foregoing examples are illustrative of the present invention, but should not to be construed as limiting thereof. The invention is defined by the following claims, with equivalents of the claims to be included therein.

### Example 45

### Effect of CoQs & an EA on In Vitro NADPH Levels

Glocose-6-Phosphate Dehydrogenase (G6PD) is an important enzyme that is widespread in mammals, and is involved in the conversion of NADP to NADPH, thereby increasing NADPH levels. An inhibition of the G6PD enzyme, thus, will be expected to result in a reduction of cellular NADPH levels, which event, in turn, will be expected to inhibit pathways that are heavily dependent on NADPH. One such pathway, the so-called One-Carbon-Pool pathway, also known as the Folate Pathway, is directly involved in the production of adenosine by addition of the C₂ and C₈ carbon atoms of the purine ring. Consequently, the inhibition of this pathway will lead to adenosine depletion.

The present invention is broadly applicable to dehdroepiandrosterones (DHEAs) and Ubiquinones (CoQs). The description of the pathways involved in the present invention are described in the Background section. The present experiment was designed to show that one DHEA and two CoQs inhibit NADPH levels. DHEA, an dehydroepiandrosterone, has already been shown to decrease levels of adenosine in various tissues. See, Examples 1 and 2 above. The fact that two CoQs are shown to lower NADPH levels to a similar extent as a dehydroepiandrosterone, let alone to a similar extent ensures that the NADPH reduction caused by the CoQs will also result in lower cellular adenosine levels or in adenosine cell depletion. Thus, in accordance with the invention, both dehydroepiandrosterones and Ubiquinones decrease levels of adenosine and, therefore, are useful as medicaments for use in the treatment of diseases where a decrease of adenosine levels or its depletion is desirable, including respiratory diseases such as asthma, bronchoconstriction, lung inflammation and allergies and the like. Both Ubiquinones and DHEA inhibit NADPH levels in a statistically significant manner, when compared to a control. Moreover, the Ubiquinone inhibits NADPH levels to a similar extent as DHEA. The present invention is broadly applicable to the use of dehydroepiandrosterones (DHEAs) and Ubiquinones (CoQs) to the treatment of respiratory and lung diseases, and other diseases associated with varying levels of adenosine, adenosine hypersensitivity, asthma, bronchoconstriction, and/or lung inflammation and allergies. The DHEA and Ubiquinones employed in the present experiments are equivalent to those described and exemplified above.

Enzymatic Assay of Purified G6PDH

The reaction mixture contained 50 mM glycyl glycine buffer, pH 7.4, 2 mM D-glucose-6-phosphate, 0.67 mM Beta-NADP, 10 mM MgCL₂ and 0.0125 units of G6PDH in a final volume of 3.0 ml. All experiments were repeated 4 times.

The control group contained 3 samples that were added no DHEA or ubiquinone. The experimental group contained a similar number of samples (3) for each concentration of DHEA or ubiquinone. One group was added DHEA (in triplicate) at different concentrations. A second group was added different concentrations of a CoQ of long side chain (in triplicate), and a third group received a CoQ of short side chain (in triplicate), both at various doses in the μM range.

The reaction was started by addition of the enzyme, and the increase in absorbance at 340 nm was measured for 5 minutes. Each data point was conducted in triplicate, and the full experiment was repeated 4 times.

Both DHEA and the ubiquinones inhibited the enzyme activity in a statistically significant manner when compared to controls. DHEA was found to inhibit by 72% in vitro the activity of purified G6PDH when compared to control. Both ubiquinones inhibited the activity of purified G6PDH in vitro by an amount that was not statistically significantly different from that of DHEA. Both DHEA and the ubiquinones inhibited the enzyme in a statistically significant manner when compared to controls. Both long chain and short chain CoQs were found to be effective inhibitors of G6PDH.

The above results clearly indicate that CoQ reduced cellular levels of NADPH to an extent similar to DHEA and consequently cellular adenosine levels, and has a therapeutic effect on diseases and conditions associated with them. The present results show that CoQs have a therapeutic effect similar to that of dehydroepiandrosterones. The pathways involved in the present invention, as described above, show the criticality of the results reported here, showing that a dehydroepiandrosterone (DHEA) and tow ubiquinones inhibit NADPH levels in a statistically significant manner. The same dehydroepiandrosterone (DHEA) was shown in Examples 1 and 2 to decrease levels of adenosine in various tissues. The two different ubiquinones employed lowered NADPH levels to a similar extent as DHEA. The NADPH reduction caused by the ubiquinones will, in the case of DHEA, result in lower cellular adenosine levels or adenosine depletion. Thus, in accordance with the invention, both dehydroepiandrosterones and ubiquinones decrease levels of adenosine and are, therefore, useful in the therapy of diseases and conditions where a decrease of adenosine levels or its depletion are desirable, including respiratory and airway diseases such as asthma, bronchoconstriction, lung inflammation and allergies, and the like.

In Examples 46 to 51, micronized anti-sense oligo targeting the adenosine A₁ receptor (EPI 2010) and micronized salmeterol (as the hydroxynaphthoate) are added in the proportions given below either dry or after predispersal in a small quantity of stabilizer, disodium dioctylsulphosuccinate, lecithin, oleic acid or sorbitan solvent to a suspension vessel containing the main bulk of the solvent. The resulting suspension is further dispersed by an appropriate mixing system using, for example, a high shear blender, ultrasonics or a mnicrofluidiser until an ultrafine dispersion is created. The suspension is then continuously recirculated to suitable filing equipment designed for cold fill or pressure filling of solvent. The suspension may be also prepared in a suitable chilled solution of stabilizer, in solvent.

| | |
|---|---|
| Active Ingredient | Target per Actuation |

Example 46: Metered Dose Inhaler

| | | |
|---|---|---|
| DHEA | 200 | mg |
| EPI 2010 | 1 | mg |
| Stabilizer | 5.0 | μg |
| Solvent (1) | 23.70 | mg |
| Solvent (2) | 61.25 | mg |

Example 47: Metered Dose Inhaler

| | | |
|---|---|---|
| DHEA-S | 200 | mg |
| EPI 2010 | 5 | mg |
| Stabilizer | 7.5 | μg |
| Solvent (1) | 23.67 | mg |
| Solvent (2) | 61.25 | mg |

Example 48: Metered Dose Inhaler

| | | |
|---|---|---|
| Ubiquinone (CoQ10) | 200 | mg |
| EPI 2010 | 30 | mg |
| Stabilizer | 25.0 | μg |
| Solvent (1) | 23.45 | mg |
| Solvent (2) | 61.25 | mg |

Example 49: Metered Dose Inhaler

| | | |
|---|---|---|
| DHEA | 600 | mg μg |
| EPI 2010 | 1.0 | mg |
| Stabilizer | 15.0 | μg |
| Solvent (1) | 23.56 | mg |
| Solvent (2) | 61.25 | mg |

Example 50: Metered Dose Inhaler

| | | |
|---|---|---|
| DHEA-S | 600 | mg |
| EPI 2010 | 5.0 | mg |
| Stabilizer | 15.0 | μg |
| Solvent (1) | 23.56 | mg |
| Solvent (2) | 61.25 | mg |

Example 51: Metered Dose Inhaler

| | | |
|---|---|---|
| Ubiquinone | 600 | mg |
| EPI 2010 | 30.0 | mg |
| Stabilizer | 25.0 | μg |
| Solvent (1) | 23.43 | mg |
| Solvent (2) | 61.25 | mg |

In the following Examples 43 to 48, the active ingredients are micronized and bulk blended with lactose in the proportions given above. The blend is filled into hard gelatin capsules or cartridges or into specifically constructed double foil blister packs (Rotadisks blister packs, Glaxo® to be adrministered by an inhaler such as the Rotahaler inhaler (Glaxo®) or in the case of the blister packs with the Diskhaler inhaler (Glaxo®).

| | |
|---|---|
| Active Ingredient | /cartridge or blister |

Example 52: Metered Dose Dry Powder Formulation

| | | |
|---|---|---|
| DHEA | 1 | mg |
| EPI 2010 | 0.05 | mg |
| Lactose Ph. Eur. | to 12.5 or 25.0 | mg |

Example 53: Metered Dose Dry Powder Formulation

| | | |
|---|---|---|
| DHEA-S | 1 | mg |
| EPI 2010 | 0.1 | mg |
| Lactose Ph. Eur. | to 12.5 or 25.0 | mg |

Example 54: Metered Dose Dry Powder Formulation

| | | |
|---|---|---|
| Ubiquinone | 1 | mg |
| EPI 2010 | 0.15 | mg |
| Lactose Ph. Eur. | to 12.5 or 25.0 | mg |

Example 55: Metered Dose Dry Powder Formulation

| | | |
|---|---|---|
| DHEA | 1 | mg |
| EPI 2010 | 0.01 | mg |
| Lactose Ph. Eur. | to 12.5 or 25.0 | mg |

Example 56: Metered Dose Dry Powder Formulation

| | | |
|---|---|---|
| DHEA-S | 1 | mg |
| EPI 2010 | 0.05 | mg |
| Lactose Ph. Eur. | to 12.5 or 25.0 | mg |

Example 57: Metered Dose Dry Powder Formulation

| | | |
|---|---|---|
| Ubiquinone | 1 | mg |
| EPI 2010 | 0.1 | mg |
| Lactose Ph. Eur. | to 12.5 or 25.0 | mg |

### Example 58

### Metered Dose Inhaler Formulation (1)

Standard 12.5 ml MDI (mitered dose inhaler) cans (Presspart Inc., Cary N.C.) are spray-coated with PTFE-FEP-polyamideimide blend (DuPont) and cured according to the vendor's standard procedure. The thickness of the coating is between approximately 1 μm and approximately 20 μm. These cans are then purged of air the valves crimped in place, and a suspension of about 68 mg of micronised beclomethasone dipropionate monohydrate and 1 mg of oligonucleotide in about 6.1 mg water and about 18.2 g P134a is filled through the valve.

### Example 59

### Metered Dose Inhaler Formulation (2)

Standard 12.5 ml MDI cans (Presspart Inc., Cary N.C.) are spray-coated with PTFE-FEP-polyamideimide blend (DuPont) and cured according to the vendor's standard procedure. The thickness of the coating is between approximately 1 μm and approximately 20 μm. These cans are then purged of air the valves crimrped in place, and about 50 mg of dehydroepiandrosterone, 1 mg of micronised oligonucleotide and 50 mg of Coenzyme Q10 in about 182 mg ethanol and about 18.2 g P134a is filled through the valve.

### Example 60

### Metered Dose Inhaler Formulation (3)

Standard 12.5 ml MDI cans (Presspart Inc., Cary N.C.) are spray-coated with PTFE-PES blend (DuPont) as a single coat and cured according to the vendor's standard procedure. The thickness of the coating is between approximately 1 μm and approximately 20 μm. These cans are then purged of air, the valves crimped in place, and a suspension of about 41.0 mg, 21.0 mg, 8.8 mg or 4.4 mg of micronised fluticasone propionate and 2 mg of micronised oligonucleotide in about 12 g P134a is filled through the valve.

### Example 61

### Metered Dose Inhaler Formulation (4)

Standard 12.5 ml MDI cans (Presspart Inc., Cary N.C.) are spray-coated with PTFE-PES blend (DuPont) as a single coat and cured according to the vendor's standard procedure. The thickness of the coating is between approximately 1 μm and approximately 20 μm. These cans are then purged of air, the valves crimped in place, and a suspension of about 8.8 mg, 22 mg or 44 mg of micronised fluticasone propionate with about 6.4 mg of micronised salmeterol xinafoate and 1 mg of micronised oligonucleotide in about 12 g P134a is filled through the valve.

### Example 62

### Metered Dose Inhaler Formulation (5)

Standard 12.5 ml MDI cans (Presspart Inc., Cary N.C.) are spray-coated with PTFE-FEP-polyamideimide blend (DuPont) and cured according to the vendor's standard procedure. The thickness of the coating is between approximately 1 μm and approximately 20 μm. These cans are then purged of air the valves crimped in place, and a suspension of about 50 mg of micronised dehydroepiandrosterone with about 6.4 mg of micronised salmeterol xinafoate and 2 mg of micronised oligonucleotide in about 12 g P134a is filled through the valve.

### Example 63

### Metered Dose Inhaler Formulation (6)

Standard 12.5 ml MDI cans (Presspart Inc., Cary N.C.) are spray-coated with PTFE-PES blend (DuPont) as a single coat and cured according to the vendor's standard procedure. The thickness of the coating is between approximately 1 μm and approximately 20 μm. These cans are then purged of air, the valves crimped in place, and a suspension of about 50 mg of micronised dehydroepiandrosterone sulfate and 2 mg of micronised oligonucleotide in about 12 g P134a is filled through the valve.

### Example 64

### Effect of CoQs & an EA on In Vitro NADPH Levels

Glocose-6-Phosphate Dehydrogenase (G6PD) is an important enzyme that is widespread in mammals, and is involved in the conversion of NADP to NADPH, thereby increasing NADPH levels. An inhibition of the G6PD enzyme, thus, will be expected to result in a reduction of cellular NADPH levels, which event, in turn, will be expected to inhibit pathways that are heavily dependent on NADPH. One such pathway, the so-called One-Carbon-Pool pathway, also known as the Folate Pathway, is directly involved in the production of adenosine by addition of the C₂ and C₈ carbon atoms of the purine ring. Consequently, the inhibition of this pathway will lead to adenosine depletion.

The present invention is broadly applicable to Epiandrosterones (EAs) and Ubiquinones (CoQs). The description of the pathways involved in the present invention are described in the Background section. The present experiment was designed to show that one EA and two CoQs inhibit NADPH levels. DHEA, an Epiandrosterone, has already been shown to decrease levels of adenosine in various tissues. See, Examples 1 and 2 above. The fact that two CoQs are shown to lower NADPH levels to a similar extent as an Epiandrosterone, let alone to a similar extent ensures that the NADPH reduction caused by the CoQs will also result in lower cellular adenosine levels or in adenosine cell depletion. Thus, in accordance with the invention, both Epiandrosterones and Ubiquinones decrease levels of adenosine and, therefore, are useful as medicaments for use in the treatment of diseases where a decrease of adenosine levels or its depletion is desirable, including respiratory diseases such as asthma, bronchoconstriction, lung inflammation and allergies and the like. Both Ubiquinones and DHEA inhibit NADPH levels in a statistically significant manner, when compared to a control. Moreover, the Ubiquinone inhibits NADPH levels to a similar extent as DHEA. The present invention is broadly applicable to the use of Epiandrosterones (EAs) and Ubiquinones (CoQs) to the treatment of respiratory and lung diseases, and other diseases associated with varying levels of adenosine, adenosine hypersensitivity, asthma, bronchoconstriction, and/or lung inflammation and allergies. The DHEA and Ubiquinones employed in the present experiments are equivalent to those described and exemplified above.

Enzymatic Assay of Purified G6PDH

The reaction mixture contained 50 mM glycyl glycine buffer, pH 7.4, 2 mM D-glucose-6-phosphate, 0.67 mM Beta-NADP, 10 mM MgCL2 and 0.0125 units of G6PDH in a final volume of 3.0 ml. All experiments were repeated 4 times.

The control group contained 3 samples that were added no DHEA or Ubiquinone. The experimental group contained a similar number of samples (3) for each concentration of DHEA or Ubiquinone. One group was added DHEA (in triplicate) at different concentrations. A second group was added different concentrations of a CoQ of long side chain (in triplicate), and a third group received a CoQ of short side chain (in triplicate), both at various doses in the μM range.

The reaction was started by addition of the enzyme, and the increase in absorbance at 340 nm was measured for 5 minutes. Each data point was conducted in triplicate, and the full experiment was repeated 4 times.

Both DHEA and the Ubiquinones inhibited the enzyme activity in a statistically significant manner when compared to controls. DHEA was found to inhibit by 72% in vitro the activity of purified G6PDH when compared to control. Both Ubiquinones inhibited the activity of purified G6PDH in vitro by an amount that was not statistically significantly different from that of DHEA. Both DHEA and the Ubiquinones inhibited the enzyme in a statistically significant manner when compared to controls. Both long chain and short chain CoQs were found to be effective inhibitors of G6PDH.

The above results clearly indicate that CoQ reduced cellular levels of NADPH to an extent similar to DHEA and consequently cellular adenosine levels, and has a therapeutic effect on diseases and conditions associated with them. The present results show that CoQs have a therapeutic effect similar to that of epiandrosterones. The pathways involved in the present invention, as described above, show the criticality of the results reported here, showing that an Epiandrosterone (DHEA) and two Ubiquinones inhibit NADPH levels in a statistically significant manner. The same epiandrosterone (DHEA) was shown in Examples 1 and 2 to decrease levels of adenosine in various tissues. The two different Ubiquinones employed lowered NADPH levels to a similar extent as DHEA. The NADPH reduction caused by the Ubiquinones will, in the case of DHEA, result in lower cellular adenosine levels or adenosine depletion. Thus, in accordance with the invention, both Epiandrosterones and Ubiquinones decrease levels of adenosine and are, therefore, useful in the therapy of diseases and conditions where a decrease of adenosine levels or its depletion are desirable, including respiratory and airway diseases such as asthma, bronchoconstriction, lung inflammation and allergies, and the like.

These are clearly superior results, which could not have been expected based on the knowledge of the art at the time of this invention. The experimental data and results provided are clearly enabling of the effect of ubiquinones on adenosine cellular levels and, therefore, on its therapeutic affect on diseases and conditions associated with them, as described and claimed in this patent.

The foregoing examples are illustrative of the present invention, and are not to be construed as limiting thereof. The invention is defined by the following claims, with equivalents of the claims to be included therein.

LENGTHY TABLE
The patent application contains a lengthy table section. A copy of the table is available in electronic form from the USPTO web site (http://seqdata.uspto.gov/?pageRequest=docDetail&DocID=US20070021360A1) An electronic copy of the table will also be available from the USPTO upon request and payment of the fee set forth in 37 CFR 1.19(b)(3).

What is claimed as novel & unobvious in United States Letters Patent is:

1. A pharmaceutical composition, comprising a pharmaceutically or veterinarily acceptable carrier or diluent, and prophylactic or therapeutic amounts of a first and second active agents;
the first active agent comprising an oligonucleotide(s) (oligo(s)) that is anti-sense to the initiation codon, the coding region, the 5′-end or the 3′-end genomic flanking regions, the 5′ and 3′ intron-exon junctions, or regions within 2 to 10 nucleotides of the junctions of one or more gene(s) encoding or to regulatory sequence(s) associated with one or more target polypeptide(s) associated with lung and/or nasal airway dysfunction, or anti-sense to the corresponding mRNA; or combinations or mires of the oligo(s); and the second active agent comprising an anti-inflammatory steroid (AIS) of chemical formula 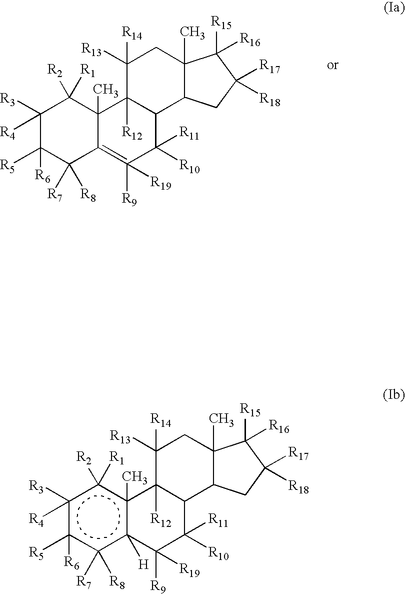
wherein R₁, R₂, R₃, R₄, R₆, R₇, R₈, R₉, R₁₀, R₁₂, R₁₃, R₁₄ and R₁₉ are independently H, OR, halogen, (C₁-C₁₀) alkyl, (C₁-C₁₀) alkene, (C₁-C₁₀) alkyne, (C₁-C₁₀) alkoxy, or two or more of R₁, R₂, R₃, R₄, R₆, R₇, R₈, R₉, R₁₀, R₁₂, R₁₃, R₁₄ and R₁₉ can be linked by combination of the atoms of C, O, N, S, P and Si to form a 3 to 15 member ring(s), in the α- and/or β-configuration;
R₅, R₆, R₁₀, and R₁₁, are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R₂₀, —OPOR₂₀R₂₁, (C₁-C₁₀) alkyl, (C₁-C₁₀) alkene, (C₁-C₁₀) allyne or OR₂₃, wherein, R₂₃ is hydrogen or SO₂OM, wherein M is selected from H, Na, sulfatide; 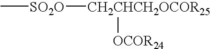
or phosphatide 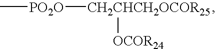
wherein R₂₄ and R₂₅, which may be the same or different, are straight or branched (C₁-C₂₀) alkyl, (C₁-C₂₀) alkene, (C₁-C₂₀) alkyne, sugar, polyethyleneglycol (PEG) or glucuronide 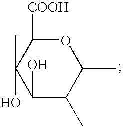
R₅ and R₆ taken together are ═O;
R₁₀ and R₁₁ taken together are ═O;
R₁₅ is (1) H, halogen, (C₁-C₁₀) alkyl, (C₁-C₁₀) alkene, (C₁-C₁₀) alkyne, or (C₁-C₁₀) alkoxy when R₁₆ is —C(O)OR₂₂, (2) H, halogen, OH, (C₁-C₁₀) alkyl, (C₁-C₁₀) alkene or (C₁-C₁₀) alkyne, when R₁₆ is halogen, OH, (C₁-C₁₀) alkyl, (C₁-C₁₀) alkene or (C₁-C₁₀) alkyne, (3) H, halogen, (C₁-C₁₀) alkyl, (C₁-C₁₀) alkenyl, (C₁-C₁₀) alkynyl, formyl, (C₁-C₁₀) alkanoyl or epoxy when R₁₆ is OH, (4) OR, SR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R₂₀ or —OPOR₂₀R₂₁ when R₁₆ is H, or R₁₅ and R₁₆ taken together are ═O;
R₁₇ and R₁₈ are independently (1) H, —OH, halogen, (C₁-C₁₀) alkyl, (C₁-C₁₀) alkene, (C₁-C₁₀) alkyne or —(C₁-C₁₀) alkoxy when R₆ is H OR, halogen, (C₁-C₁₀) alkyl or —C(O)OR₂₂, (2) H, (C₁-C₁₀alkyl)ₙ amino, (C₁-C₁₀ alkene)ₙ amino, (C₁-C₁₀alkyne)ₙ amino, ((C₁-C₁₀) alkyl)ₙ amino-(C₁-C₁₀) alkyl, ((C₁-C₁₀) alkene)ₙ amino-(C₁-C₁₀) alkyl, ((C₁-C₁₀) alkyne)ₙ amino-(C₁-C₁₀) alkyl, ((C₁-C₁₀) alkyl)ₙ amino-(C₁-C₁₀) alkene, ((C₁-C₁₀) alkene)ₙ amino-(C₁-C₁₀) alkene, ((C₁-C₁₀) alkyne)ₙ amino-(C₁-C₁₀) alkene, ((C₁-C₁₀) alkyl)ₙ amino-(C₁-C₁₀) alkyne, ((C₁-C₁₀) alkene)ₙamino-(C₁-C₁₀) alkyne, ((C₁-C₁₀) alkyne)ₙ amino-(C₁-C₁₀) alkyne, (C₁-C₁₀) alkoxy, hydroxy-(C₁-C₁₀) alkyl, hydroxy-(C₁-C₁₀) alkene, hydroxy-(C₁-C₁₀) alkyne, (C₁-C₁₀) alkoxy-(C₁-C₁₀) alkyl, (C₁-C₁₀) alkoxy-(C₁-C₁₀) alkene, (C₁-C₁₀) alkoxy-(C₁-C₁₀) alkyne, (halogen)ₘ (C₁-C₁₀) alkyl, (halogen)ₘ (C₁-C₁₀) alkene, (halogen)ₘ (C₁-C₁₀) alkyne, (C₁-C₁₀) alkanoyl formyl, (C₁-C₁₀) carbalkoxy or (C₁-C₁₀) alkanoyloxy when R₁₅ and R₁₆ taken together are ═O, (3) R₁₇ and R₁₈ taken together are ═O; (4) R₁₇ and R₁₈ taken together with the carbon to which they are attached form a 3-6 member ring containing 0 or 1 oxygen atom; or (5) R₁₅ and R₁₇ taken together with the carbons to which they are attached form an epoxide ring; R₂₀ and R₂₁ are independently OH, pharmaceutically acceptable ester or pharmaceutically acceptable ether; R₂₂ is H, (halogen)ₘ (C₁-C₁₀) alkyl, (halogen)ₘ(C₁-C₁₀) alkene, (halogen)ₘ (C₁-C₁₀) alkyne, (C₁-C₁₀) alkyl, (C₁-C₁₀) alkene or (C₁-C₁₀) alkyne; n is 0, 1 or 2; and m is 1, 2 or 3;
or pharmaceutically or veterinarily acceptable salts thereof; and/or
a ubiquinone of the chemical formula 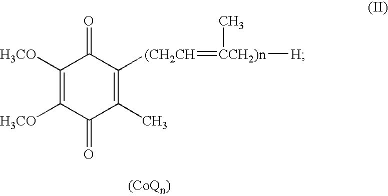
wherein n=1 to 12, or pharmaceutically or veterinarily acceptable salts thereof; the first and second agents being present in amounts effective for reducing or depleting levels of, or reducing sensitivity to, adenosine, reducing levels of adenosine receptors, producing bronchodilation, increasing levels of ubiquinone or lung surfactant in a subject's tissue (s), or treating bronchoconstriction, lung inflammation or lung allergies or a respiratory or lung disease or condition.

2. The composition of claim 1, wherein the oligo contains up to about 15% A.

3. The composition of claim 1, wherein the oligo(s) of the first active agent is (are) anti-sense to the initiation codon, the coding region, the 5′-end or the 3′-end genomic flanking regions, the 5′ or 3′ intron-exon junctions, and regions within 2 to 10 nucleotides of the junctions of at least one oncogene(s) or a gene(s) encoding, or regulating expression of, a target polypeptide(s) associated with lung and/or nasal airway dysfunction or cancer, is (are) anti-sense to the corresponding mRNA(s). Multiple target anti-sense oligo(s) (MTAs) or combinations thereof; the polypeptides comprising peptide factors and transmitters, antibodies, cytokines or chemokines, enzymes, binding proteins, adhesion molecules, their receptors, or malignancy associated proteins.

4. The composition of claim 3, wherein the oligo(s) is (are) anti-sense to the initiation codon, the coding region, the 5′-end or the 3′-end genomic flanking regions, the 5′ or 3′ intron-exon junctions, or regions within 2 to 10 nucleotides of the junctions of at least one oncogene(s) or a gene(s) encoding, or regulating expression of, a target polypeptide(s) associated with lung and/or nasal airway dysfunction or is (are) anti-sense to the oncogene mRNA, or the corresponding mRNA; or MTAs or combinations thereof; wherein the polypeptides comprise of transcription factors, stimulating or activating peptide factors, cytokines, cytokine receptors, chemokines, chemokine receptors, adenosine receptors, bradykinin receptors, endogenously produced specific or non-specific enzymes, immunoglobulins or antibodies, antibody receptors, central nervous system (CNS) or peripheral nervous or non-nervous system receptors, CNS or peripheral nervous or non-nervous system peptide transmitters, adhesion molecules, defensins, growth factors, vasoactive peptides and receptors, binding proteins, or malignancy associated proteins.

5. The composition of claim 4, wherein the encoded polypeptide(s) comprise(s) one or more adenosine receptors A₁, A₂ₐ, A_{2b} or A₃, bradykinin receptors B1 or B2, NFκB Transcription Factor, Interleukin-8 Receptor (IL-8 R), Interleukin 5 Receptor (IL-5 R), Interleukin 4 Receptor (IL-4 R), Interleukin 3 Receptor (IL-3 R), Interleukin-1β (IL-1β), Interleukin 1β Receptor (IL-1βR), Eotaxin, Tryptase, Major Basic Protein, β2-adrenergic Receptor Kinase, Endothelin Receptor A, Endothelin Receptor B, Preproendothelin, Bradykinin B2 Receptor, IgE High Affinity Receptor, Interleukin 1 (IL-1), Interleukin 1 Receptor (IL-1R), Interleukin 9 (IL-9), Interleukin-9 Receptor (IL-9 R), Interleukin 11 (IL-11), Interleukin-11 Receptor (IL-11 R), Inducible Nitric Oxide Synthase, Cyclo-oxygenase-1 (COX-1), Cyclo-oxygenase-2 (COX-2), Intracellular Adhesion Molecule 1 (ICAM-1) Vascular Cellular Adhesion Molecule (VCAM), Rantes, Endothelial Leukocyte Adhesion Molecule (ELAM-1), Monocyte Activating Factor, Neutrophil Chemotactic Factor, Neutrophil Elastase, Defensin 1, 2 and 3, Muscarinic Acetylcholine Receptors, Platelet Activating Factor, Tumor Necrosis Factor α, 5-lipoxygenase, Phosphodiesterase IV, Substance P, Substance P Receptor, Histamine Receptor, Chymase, CCR-1 CC Chemokine Receptor, CCR-2 CC Chemokine Receptor, CCR-3 CC Chemokine Receptor, CCR-4 CC Chemokine Receptor, CCR-5 CC Chemokine Receptor, Prostanoid Receptors, GATA-3 Transcription Factor, Neutrophil Adherence Receptor, MAP Kinase, Interleukin-9 (IL-9), NFAT Transcription Factors, STAT 4, MIP-1α, MCP-2, MCP-3, MCP-4, Cyclophillins, Phospholipase A2, Basic Fibroblast Growth Factor, Metalloproteinase, CSBP/p38 MAP Kinase, Tryptase Receptor, PDG2, Interleukin-3 (IL-3), Interleukin-1β (IL-1β), Cyclosporin A-Binding Protein, FK5-Binding Protein, α4β1 Selectin, Fibronectin, α4β7 Selectin, Mad CAM-1, LFA-1 (CD11a/CD18), PECAM-1, LFA-1 Selectin, C3bi PSGL-1, E-Selectin, P-Selectin, CD-34, L-Selectin, p150, 95, Mac-1 (CD11b/CD18), Fucosyl transferase, VLA-4, CD-18/CD11a, CD11b/CD18, ICAM2 and ICAM3, C5a, CCR3 (Eotaxin Receptor), CCR1, CCR2, CCR4, CCR5, LTB-4, AP-1 Transcription Factor, Protein kinase C, Cysteinyl Leukotriene Receptor, Tachychinnen Receptors (tach R), IkB Kinase 1 & 2, STAT 6, c-mas or NF-Interleukin-6 (NF-IL-6).

6. The composition of claim 4, wherein the encoded polypeptide(s) comprise(s) a H2A histone family member N, Tubulin, beta polypeptide, ELL gene (11-19 lysine-rich leukemia gene); 7-dehydrocholesterol reductase, ADP-ribosylation factor-like 7, Karyopherin alpha 2 (RAG cohort 1, importin alpha 1), EST (AI038433), EST (AI122689), EST (AI092623), ESTs (AI095492), ESTs (AI138216), ESTs (AI128305), ESTs (AI125228), ESTs (AI041482), ESTs (AI051839), Homo sapiens mRNA; cDNA DKFZp434A1716, ESTs (AI096522), ESTs (AI122807), ESTs (AI041212), EST (AI125651), Enolase 1, (alpha), EST (AI024215), EST (AI034360), Homo sapiens nRNA; cDNA DKFZp564HO764, Homo sapiens mRNA for KIAA1363 protein, partial cds, Potassium voltage-gated channel, shaker-related subfamily, beta member 2, ER-associated DNAJ; ER-associated Hsp40 co-chaperone; hDj9; ERj3, ESTs, Weakly similar to p38 protein [H. sapiens] (AA906703), CGI-142, ESTs (AA463249), Homo sapiens clone 25058 mRNA sequence ESTs (R49144), Squamous cell carcinoma antigen 1, ESTs (AA425700), Myosin X, ESTs (AA459692), Epithelial protein lost in neoplasm beta, CD44 antigen (homing function and Indian blood group system), Coagulation factor III (thromboplastin, tissue factor), ESTs (AA909635), Adducin 1 (alpha), 5′ Nucleotidase (CD73), ESTs, moderately similar to semaphorin C [M. musculus] (AA293300), ESTs (AA278764), ESTs (AA678160), Calmodulin 2 (phosphorylase kinase, delta), ESTs (R42770), Chloride intracellular channel 1, High-mobility group (nonhistone chromosomal) protein 17, Ubiquitin carrier protein, Tubulin, alpha 1 (testis specific), Transglutaminase 2 (C polypeptide, protein-glutamine-gamma-glutamyltransferase), Sparc/osteonectin, cwcv and kazal-like domains proteoglycan (testican), Proteasome (prosome, macropain) 26S subunit, non-ATPase, 2, TubuLin, beta polypeptide, Filamin B, beta (actin-binding protein-278), Stanniocalcin, Low density lipoprotein receptor (familial hypercholesterolemia), Plectin 1, intermediate filament binding protein, 500 kD, S100 calcium-binding protein A2, Immediate early response 3, Calpain, large polypeptide L2, Pleckstrin homology-Like domain, family A, member 1, Melanoma adhesion molecule, CD44 antigen (homing function and Indian blood group system), Programmed cell death 5, Hexokinase 1, Vascular endothelial growth factor, Integrin, alpha 2 (CD49B, alpha 2 subunit of VLA-2 receptor), Calumenin, Syntaxin 11, Diphtheria toxin receptor (heparin-binding epidermal growth factor-like growth factor), Fn14 for type I transmembrane protein, Nef-associated factor 1, High-mobility group (nonhistone chromosomal) protein isoforms I and Y, Catechol-O-methyltransferase, C-terminal binding protein 1, Collagen, type XVII, alpha 1, ESTs (N58473), Farnesyl-diphosphate farnesyltransferase 1 RNA helicase-related protein, Interferon stimulated gene (20 kD), Steroid-5-alpha-reductase, alpha polypeptide 1 (3-oxo-5 alpha-steroid delta 4-dehydrogenase alpha 1), Prostaglandin-endoperoxide synthase 2 (prostaglandin G/H synthase and cyclooxygenase), Laminin, alpha 3 (nicein (150 kD), kalinin (165 kD), BM600 (150 kD), epilegrin), Collagen, type XVII, alpha 1, Keratin 18, Heparan sulfate (glucosamine) 3-O-sulfotransferase 1, Tubulin, alpha 2, Adenylyl cyclase-associated protein, Forkhead box D1, Cathepsin C, ESTs, Highly similar to AF151802_{—}1 CGI-44 protein [H. sapiens] (T74688), Ribonucleotide reductase M2 polypeptide, Laminin, gamma 2 (nicein (100 kD), kalinin (105 kD), BM600 (100 kD), Herlitz junctional epidermolysis bullosa)), Homo sapiens mRNA; cDNA DKFZp586P1622 (from clone DKFZp586P1622), ESTs, Weakly similar to/prediction (AA284245), or Lactate dehydrogenase A.

7. The composition of claim 1, wherein one or more As of the first active agent is(are) substituted by a universal base comprising a heteroaromatic base that binds to thymidine or uridine but has antagonist activity or less than about 0.3 of the adenosine agonist or antagonist activity at the adenosine A₁, A₂ₐ, A_{2b} or A₃ receptors.

8. The composition of claim 7, wherein the heteroaromatic base(s) comprise(s) pyrimidines or purines, which may be substituted by O, halo, NH₂, SH, SO, SO₂, SO₃, COOH, branched or fused primary or secondary amino, alkyl, alkenyl, alkynyl, cycloalkyl, heterocycloalkyl, aryl, heteroaryl, alkoxy, alkenoxy, acyl, cycloacyl arylacyl, alkynoxy, cycloalkoxy, aroyl, arylthio, arylsulfoxyl, halocycloalkyl, alkylcycloalkyl, alkenylcycloalkyl, alkynylcycloalkyl, haloaryl, alkylaryl, alkenylaryl, alkynylaryl, arylalkyl, arylalkenyl, arylalkynyl, arylcycloalkyl, all of which may be further substituted by O, halo, NH₂, primary, secondary or tertiary amine, SH, SO, SO₂, SO₃, cycloalkyl, heterocycloalkyl or heteroaryl.

9. The composition of claim 7, wherein the purines are substituted at positions 1, 2, 3, 6, and/or 8, the pyrimidines are substituted at positions 2, 3, 4, 5 and/or 6, and the purines and pyrimidines have the chemical formula 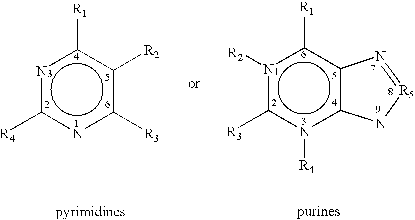
wherein R¹, R², R³, R⁴ and R⁵ are independently H, alkyl, alkenyl or alkynyl and R³ is H, aryl, dicycloalkyl, dicycloalkenyl, dicycloalkynyl, cycloalkyl, cycloalkenyl, cycloalkynyl, O-cycloalkyl, O-cycloalkenyl, O-cycloalkynyl, NH₂-alkylamino-ketoxyalkyloxy-aryl, or mono or dialkylaminoalkyl-N-alkylamino-SO₂aryl, and R4 and R5 are independently R1 and together are R3, and the pyrimidines and purines optionally comprise theophylline, caffeine, dyphylline, etophylline, acephylline piperazine, bamifylline, enprofylline or xanthine.

10. The composition of claim 9, wherein the universal base of the first active agent comprises 3-nitropyrrole-2′-deoxynucleoside, 5-nitro-indole, 2-deoxyribosyl-(5-nitroindole), 2-deoxyribofuranosyl-(5-nitroindole), 2′-deoxyinosine, 2′-deoxynebularine, 6H, 8H-3,4 diydropyrimido[4,5-c]oxazine-7-one or 2-amino-6-methoxyaminopurine.

11. The composition of claim 1, wherein if present in the first active agent(s), one or more methylated cytocine(s) (^{m}C) is(are) substituted for a C in or to form one or more CpG dinucleotide(s).

12. The composition of claim 1, wherein one or more mononucleotide(s) of the first active agent(s) is(are) linked or modified by one or more of methylphosphonate, 5′-N-carbamate, phosphotriester, phosphorothioate, phosphorodithioate, boranophosphate, formacetal, thioformacetal, thioether, carbonate, carbamate, sulfate, sulfonate, sulfamate, sulfonamide, sulfone, sulfite, sulfoxide, sulfide, hydroxylamine, methylene(methylmito) (MMI), methoxymethyl (MOM), methoxyethyl (MOE), methyleneoxy(methylimino) (MOMI), 2′-O-methyl, phosphoramidate, or C-5 substituted residues.

13. The composition of claim 12, wherein one or more mononucleotide residue(s) of the first active agent(s) are linked by phosphorothioate residues.

14. The composition of claim 1, wherein the anti-sense oligo of the first active agent(s) comprise(s) about 7 to about 60 mononucleotides.

15. The composition of claim 1, wherein the anti-sense oligo of the first active agent(s) comprise(s) fragments 1, 3, 5, 7 and 8 to 2498 (SEQ ED NOS: 1 through 2498).

16. The composition of claim 1, wherein the anti-sense oligo of the first active agent(s) is(are) operatively linked to, or complexed with, a cell internalized or up-taken agent(s) or a cell targeting agent(s).

17. The composition of claim 15, wherein the cell internalized or up-taken agent comprises transferrin, asialoglycoprotein or streptavidin, and the cell targeting agent comprises a prokaryotic or eukaryotic vector or plasmid.

18. The composition of claim 1, wherein the oligo contains up to about 10% A.

19. The composition of claim 1, wherein the oligo(s) of the first active agent(s) is(are) hybridized to a ribonucleic acid or a deoxyribonucleic acid and delivered as a double stranded agent.

20. The composition of claim 1, wherein the carrier or diluent comprises a gaseous, liquid, or solid carrier or diluent, and the active agents are present in an amount of about 0.01 to about 99.99 w/w of the composition.

21. The composition of claim 20, further comprising an agent selected from other therapeutic agents, surfactants, flavoring or coloring agents, fillers, volatile oils, buffering agents, dispersants, RNA inactivating agents, anti-oxidants, flavoring agents, propellants or preservatives.

22. The composition of claim 21, wherein the other therapeutic or bioactive agent(s) is (are) selected from analgesics, pre-menstrual medications, menopausal agents, anti-aging agents, anti-anxyolytic agents, mood disorder agents, anti-depressants, anti-bipolar mood agents, anti-schyzophrenic agents, anti-cancer agents, alkaloids, blood pressure controlling agents, muscle relaxants, steroids, soporific agents, anti-ischemic agents, anti-arrythmic agents, contraceptives, vitamins, minerals, tranquilizers, neurotransmitter regulating agents, wound healing agents, anti-angyogenic agents, cytokines, growth factors, B-adrenergic receptor agonists, anti-metastatic agents, antacids, anti-histaminic agents, anti-bacterial agents, anti-viral agents, anti-gas agents, appetite suppressants, sun screens, emollients, skin temperature lowering products, radioactive phosphorescent or fluorescent contrast diagnostic or imaging agents, libido altering agents, bile acids, laxatives, anti-diarrheic agents, skin renewal agents, hair growth agents, analgesics, pre-menstrual medications, anti-menopausal agents, hormones, anti-aging agents, anti-anxiolytic agents, nociceptic agents, mood disorder agents, anti-depressants, anti-bipolar mood agents, anti-schizophrenic agents, anti-cancer agents, alkaloids, blood pressure controlling agents, other hormones, other anti-inflammatory agents, agents for treating arthritis, burns, wounds, chronic bronchitis, chronic obstructive pulmonary disease (COPD), inflammatory bowel disease such as Crohn's disease, ulcerative colitis, autoimmune disease, or lupus erythematosus, muscle relaxants, soporific agents, anti-ischemic agents, anti-arrhythmic agents, contraceptives, vitamins, minerals, tranquilizers, neurotransmitter regulating agents, wound and burn healing agents, anti-angiogenic agents, cytokines, growth factors, anti-metastatic agents, antacids, anti-histaminic agents, anti-bacterial agents, anti-viral agents, anti-gas agents, agents for reperfusion injury, counteracting appetite suppressants, sun screens, emollients, skin temperature lowering products, radioactive phosphorescent or fluorescent contrast diagnostic or imaging agents, libido altering agents, bile acids, laxatives, anti-diarrheic agents, skin renewal agents or hair growth agents.

23. The composition of claim 22, wherein the surfactant comprises surfactant protein A, surfactant protein B, surfactant protein C, surfactant protein D and surfactant Protein E, di-saturated phosphatidyl choline (other than dipalmitoyl), dipalmitoyl phosphatidyl choline, phosphatidyl choline, phosphatidyl glycerol, phosphatidyl inositol, phosphatidyl ethanolamine, phosphatidyl serine; phosphatidic acid, ubiquinones, lysophosphatidyl ethanolamine, lysophosphatidyl choline, palmitoyl-lysophosphatidyl choline, dehydroepiandrosterone, dolichols, sulfatidic acid, glycerol-3-phosphate, dihydroxyacetone phosphate, glycerol glycero-3-phosphocholine, dihydroxy acetone, palmitate, cytidine diphosphate (CDP) diacyl glycerol, CDP choline, choline, choline phosphate; natural or artificial lamellar bodies as carrier surfactant vehicles, omega-3 fatty acids, polyenic acid, polyenoic acid, lecithin, palmitinic acid, non-ionic block copolymers of ethylene or propylene oxides, polyoxypropylene, monomeric or polymeric, polyoxyethylene, monomeric and polymeric, poly (vinyl amine) with dextran and/or alkanoyl side chains, Brij 35, Triton X-100 or synthetic surfactants ALEC, Exosurf, Survan or Atovaquone.

24. The composition of claim 1, comprising one or more oligo(s), an anti-inflammatory steroid(s) of formula (Ia) or (Ib), a steroid, a surfactant and a carrier or diluent for the oligo.

25. The composition of claim 1, wherein the second active agent comprises CoQₙ, wherein n is 1 to 10.

26. The composition of claim 1, wherein the second active agent comprises CoQₙ, wherein n is 6 to 10.

27. The composition of claim 1, wherein the second active agent comprises CoQₙ wherein n is 10.

28. The composition of claim 1, wherein the second active agent comprises an anti-inflammatory steroid (AIS) of formula (Ia) selected from dehydroepiandrosterone, wherein R and R¹ are H and the broken line represents a double bond, 16-alpha bromodehydroepiandrosterone wherein R is Br, R¹ is H and the broken line represents a double bond, 16-alphafluorodehydroepiandrosterone wherein R is F, R¹ is H and the broken line represents a double bond, etiocholanolone, wherein R and R¹ are each hydrogen and the broken line represents a single bond, dehydroepiandrosterone sulfate, wherein R is H, R1 is SO₂OM and M is a sulfatide group as defined above, and the broken line represents a double bond, the compound of formula (Ia), R is halogen selected from Br, Cl or F, R1 is H, and the broken line represents a double bond, 16-alpha-fluorodehydro-epiandrosterone, or pharmaceutically or veterinarily acceptable salts thereof.

29. The composition of claim 1, wherein the oligo(s) of the first agent contains up to about 5% A.

30. The composition of claim 1, wherein the oligo(s) of the first agent is A free.

31. The composition of claim 1, wherein the second active agent comprises an anti-inflammatory steroid (AIS) of formula (Ib), wherein R¹⁵ and R¹⁶ together are ═O; R⁵ is —OH; R⁵ is —OSO₂R²⁰; R¹⁵ and R²⁰ together is H; or pharmaceutically or veterinarily acceptable salts thereof.

32. The composition of claim 1, wherein the second active agent comprises an AIS selected from budesonide, testosterone, progesterone, fluticasone, beclomethasone, prednisone, momethasone, estrogen, dexamethasone, hydrocortisone, triamcinolone, flunisolide, methylprednisolone prednisone, hydrocortisone, or analogues thereof.

33. The composition of claim 1, wherein the active agents are present in an amount of about 0.01 to about 99.99 w/w of the composition.

34. The composition of claim 1, wherein the second active agent comprises an anti-inflammatory steroid (AIS) selected from 21-acetoxypregnenolone ((3β)-21-(acetyloxy)-3-hydroxypregn-5-en-20-one); alclometasone ((7α, 11β, 16α)-7-Chloro-11,17,21-trihydroxy-16-methylpregna-1,4-diene-3,20-dione), or its 17,21-dipropionate form (C₂₈H₃₇ClO₇); algestone ((16α)-16,17-dihydroxypregn-4-ene-3,20-dione), its cyclic acetal with acetone form (C₂₄H₃₄O₄), or its 16α-methyl ether form (C₂₂H₃₂O₄); amcinonide ((11β, 16α)-21-(acetyloxy)-16,17-[cyclopentylidenebis(oxy)]-9-fluoro-11-hydroxypregna-1,4-di-ene-3,20-dione); beclomethasone ((11β,16β)-9-chloro-11,17,21-trihydroxy-16-methylpregna-1,4-diene-3,20-dione), its dipropionate form (C₂₈H₃₇ClO₇), or its monopropionate form; betamethasone ((11β,16β)-9-fluoro-11,17,21-trihydroxy-16-methylpregna-1,4-diene-3,20-dione), its 21-acetate form (C₂₄H₃₁FO₆), its 21-adamantoate form (C₃₃H₄₃FO₆), its 17-benzoate form (C₂₉H₃₃FO₆), its 17,21-dipropionate form (C₂₈H₃₇FO₇), its 17-valerate form (C₂₇H₃₇FO₆), or its 21-phospate disodium salt form (C₂₂H₂₈FNa₂O₈P); budesonide ((11β,16α)-16,17-[butylidenebis(oxy)]-11,21-dihydropregna-1,4-diene-3,20-dione); chloroprednisone ((6α)-chloro-17,21-dihydroxypregna-1,4-diene-3,11,20-trione), or its 21-acetate from (C₂₃H₂₇ClO₆); ciclesonide; clobetasol ((11β,16β)-21-chloro-9-fluoro-11,17-dihydroxy-16-methylpregna-1,4-diene-3,20-dione), or its 17-propionate form (C₂₅H₃₂ClFO₅); clobetasone ((16β)-21-chloro-9-fluoro-17-hydroxy-16-methylpregna-1,4-diene-3,11,20-trione), or its 17-butyrate form (C₂₆H₃₂ClFO₅); clocortolone ((6α,11β,16α)-9-chloro-6-fluoro-11,21-dihydroxy-16-methylpregna-1,4-diene-3,20-dione), its 21-acetate form (C₂₄H₃₀ClFO₅), or its 21-pivalate form (C₂₇H₃₆ClFO₅); cloprednol ((11β)-6-chloro-11,17,21-trihydroxypregna-1,4,6-triene-3,20-dione); coroxon (phosphoric acid 3-chloro-4-methyl-2-oxo-2H-1-benzopyran-7-yl diethyl ester); cortisone (17,21-dihydroxypregn-4-ene-3,11,20-trione), its 21-acetate form (C₂₃H₃₀O₆), or its 21-cyclopentanepropionate form (C₂₉H₄₀O₆); cortivazol ((11β, 16α)-21-(acetyloxy)-11,17-dihydroxy-6,16-dimethyl-2′-phenyl-2′H-pregna-2,4,6-trieno[3,2-c]pyrazol-20-one); deflazacort ((11β,16β)-21-(acetyloxy)-11-hydroxy-2′-methyl-5′H-pregna-1,4-dieno[17,16-d]oxazole-3,20-(dione); desonide ((11β,16α)11,21-dihydroxy-16,17-[(1-methylethylidene)bis(oxy)]pregna-1,4-diene-3,20-dione); desoximetasone ((11β,16α)-9-fluoro-11,21-dihydroxy-16-methylpregna-1,4-diene-3,20-dione); dexamethasone ((11β,16α)-9-fluoro-11,17,21-trihydroxy-16-methylpregna-1,4-diene-3,20-dione), its 21-acetate form (C₂₄H₃₁FO₆), its 21-3,3-dimethylbutyrate) form (C₂₈H₃₉FO₆; Chemerda et al., U.S. Pat. No. 2,939,873), its 21-diethylaminoacetate form (C₂₉H₄₁FNO₆), its 21-isonicotinate form (C₂₈H₄₁FNO₆), its 17,21-dipropionate form (C₂₈H₃₇FNO₆), or its 21-palmitate form (C₃₈H₅₉FO₆); diflorasone ((6α,11β,16β)-6,9-difluoro-11,17,21-trihydroxy-16-methylpregna-1,4-diene-3,20-dione), or its diacetate form (C₂₆H₃₂F₂O₇); diflucortolone ((6α,11β,16α)-6,9-difluoro-11,21-dihydroxy-16-methylpregna-1,4-ene-3,20-dione), or its 21-valerate form (C₂₇H₃₆F₂O₅); difluprednate ((6α,11β)-21-acetyloxy)-6,9-difluoro-11-hydroxy-17-(1-oxobutoxy)pregna-1,4-diene-3,20-dione); enoxolone ((3β,20β)-3-hydroxy-11-oxoolean-12-en-29-oic acid), or its 18α-hydrogen form; fluazacort ((11β,16β)-21-acetyloxy)-9-fluoro-11-hydroxy-2′-methyl-5′H-pregna-1,4-dieno[17,16-d]oxazole-3,20-dione); flucloronide ((6α,11β,16α)-9,11-dichlro-6-fluoro-21-hydroxy-16,17-[(1-methylethylidene)bis(oxy)]-pregna-1,4-diene-3,20-dione); flumethasone ((6α,11β,16α)-6,9-difluoro-11,17,21-trihydroxy-16-methylpregna-1,4-diene-3,20-dione), its 21-acetate form (C₂₄H₃₀F₂O₆), or its 21-pivalate form (C₂₇H₃₆F₂O₆); flunisolide ((6α,11β,16α)-6-fluoro-11,21-dihydroxy-16,17-[(1-methylethylidene) bis(oxy)]pregna-1,4-diene-3,20-dione), or its 21-acetate form (C₂₆H₃₃FO₇); fluocinolone acetate((6α,11β,16α)-6,9-difluoro-11,21-dihydroxy-16,17-[(1-methylethylidene)bis(oxy)]-pregna-1,4-diene-3,20-dione); fluocinonide ((6α,11β,16α)-21-(acetyloxy)-6,9-difluoro-11-hydroxy-16,17-[(1-methylethylidene)bis(oxy)]-pregna-1,4-diene-3,20-dione); fluocortin butyl ((6α,11β,16α)-6-fluoro-11-hydroxy-16-methyl-3,20-dioxopregna-1,4-dien-21-oic acid butyl ester); fluocortolone ((6α,11β,16α)-6-fluoro-11,21-dihydroxy-16-methylpregna-1,4-diene-3,20-dione), its 21-acetate form (C₂₄H₃₁FO₅), its 21-hexanoate form (C₂₈H₃₉FO₅), or its 21-pivalate form (C₂₂H₃₇FO₅); fluorometholone ((6α,11β)-9-fluoro-11,17-dihydroxy-6-methylpregana-1,4-diene-3,20-dione), or its 17-acetate form (C₂₄H₃₁FO₅); fluperolone acetate([11β,17α,17(S)]-17-[2-(acetyloxy)-1-oxopropyl]-9-fluoro-11,17-dihydroxyandrosta-1,4-dien-3-one); fluprednidene acetate((11β)-21-(acetyloxy)-9-fluoro-11,17-dihydroxy-16-methylenepregna-1,4-diene-3,20-dione); fluprednisolone ((6α,11β)-6-fluoro-11,17,21-trihydroxypregna-1,4-diene-3,20-dione), or its 21-acetate form (C₂₃H₂₉FO₆); flurandrenolide ((6α,11β, 16α)-6-fluoro-11,21-dihydroxy-16,17-[(1-methylethylidene)bis(oxy)]pregn-4-ene-3,20-dione); fluticasone propionate ((6α,11β,16α,17α)-6,9-difluoro-11-hydroxy-16-methyl-3-oxo-17-(1-oxopropoxy)androsta-1,4-diene-17-carbothioic acid S-(fluoromethyl) ester); formocortal ((11β,16α)-21-(acetyloxy)-3-(2-chloroethoxy)-9-fluoro-11-hydroxy-16,17-[(1-methylethylidene)bis(oxy)]-20-oxopregna-3,5-diene-6-carboxaldehyde); halcinonide ((11β,16α)-21-chloro-9-fluoro-11-hydroxy-16,17-[(1-methyethylidene)bis(oxy)]pregn-4-ene-3,20-dione); halobetasol propionate (6α,11β,16β)-21-chloro-6,9-difluoro-11-hydroxy-16-methyl-17-(1-oxopropoxy)pregna-1,4-diene-3,20-dione); halometasone ((6α,11β,16α)-2-chloro-6,9-difluoro-11,17,21-trihydroxy-16-methylpregna-1,4-diene-3,20-dione), or its monohydrate form (C₂₂H₂₇ClF₂O₅.H₂O); halopredone acetate((6β,11β)-17,21-bis(acetyloxy)-2-bromo-6,9-difluoro-11-hydroxypregna-1,4-diene-3,20-dione); hydrocortamate (N,N-diethylglycine (11β)-11,17-dihydroxy-3,20-dioxopregn-4-en-21-yl ester), or its hydrochloride form (C₂₇H₄₁NO₆.HCl); hydrocortisone ((11β)-11,17,21-trihydroxypregn-4-ene-3,20-dione), its 21-acetate form (C₂₃H₃₂H₆), its 17-butyrate form (C₂₅H₃₆O₆), its 21-phosphate disodium salt form (C₂₁H₂₉Na₂O₈P), its 21-sodium succinate form (C₂₅H₃₃NaO₈), its 17-valerate form (C₂₆H₃₈O₆), or its cypionate form; loteprednol etabonate ((11β, 17α)-17-[(ethoxycarbonyl)oxy]-11-hydroxy-3-oxoandrosta-1,4-diene-17-carboxylic acid chloromethyl ester); mazipredone ((11β)-11,17-dihydroxy-21-4-methyl-1-piperazinyl)pregna-1,4-diene-3,20-dione), or its hydrochloride form (C₂₆H₃₈N₂O₄.HCl); medrysone ((6α, 11β)-11-hydroxy-6-methylpregn-4-ene-3,20-dione); meprednisone ((16β)-17,21-dihydroxy-16-methylpregna-1,4-diene-3,11,20-trione), or its 21-acetate form (C₂₄H₃₀O₆); methylprednisolone ((6α,11β)-11,17,21-trihydroxy-6-methylpregna-1,4-diene-3,20-dione; Sebek and Spero, U.S. Pat. No. 2,897,218, and Gould, U.S. Pat. No. 3,053,832), its 21-acetate form (C₂₄H₃₂O₆), its 21-phosphate disodium salt form (C₂₂H₂₉Na₂O₈P), its 21-succinate sodium salt form (C₂₆H₃₃NaO₈), or its aceponate form (C₂₇H₃₆O₇); mometasone furoate ((11β,16α)-9,21-dichloro-17-[(2-furanylcarbonyl)oxy]-11-hydroxy-16-methylpregna-1,4-diene-3,20-dione); paramethasone ((6α,11β,16α)-6-fluoro-11,17,21-trihydroxy-16-methylpregna-1,4-diene-3,20-dione), its 21-acetate form (C₂₄H₃₁FO₆), its disodium phosphate form, or a mixture of its 21-acetate and disodium phosphate form; prednicarbate ((11β)-17[(ethoxycarbonyl)oxy]-11-hydroxy-21-(1-oxopropoxy)pregna-1,4-diene-3,20-dione);prednisolone ((11β)-11,17,21-trihydroxypregna-1,4-diene-3,20-dione), its 21-acetate form (C₂₃H₃₀O₆), its 21-tert-butylacetate form (C₂₇H₃₈O₆; Sarrett), its 21-hydrogen succinate form (C₂₅H₃₂O₈), its 21-succinate sodium salt form (C₂₅H₃₁NaO₈), its 21-stearoylgylcolate form (C₄₁H₆₄O₈), its 21-m-sulfobenzoate sodium salt form (C₂₈H₃₁NaO₉S; (11β)-11,17-dihydroxy-21-[(3-sulfobenzoyl)oxy]pregna-1,4-diene-3,20-dione monosodium salt), or its 21-trimethylacetate form (C₂₆H₃₆O₆); prednisolone 21-diethylaminoacetate (N,N-diethylglycine (11β)-11,17-dihydroxy-3,20-dioxopregna-1,4-dien-21-yl ester; British Patent No. 862,370), or its hydrochloride form (C₂₇H₃₉NO₆.HCl); prednisolone sodium phosphate (11,17-dihydroxy-21-(phosphonooxy)pregna-1,4-diene-3,20-dione disodium salt); prednisone (17,21-dihydroxypregna-1,4-diene-3,11,20-trione), or its 21-acetate form (C₂₃H₂₈O₆); prednival ((11β)-11,21 dihydroxy-17-[(1-oxopentyl)oxy]pregna-1,4-diene-3,20-dione;), or its 21-acetate form (C₂₈H₃₈O₇); prednylidene ((11β)-11,17,21-trihydroxy-16-methylenepregna-1,4-diene-3,20-dione), or its 21-diethylaminoacetate hydrochloride form (C₂₈H₃₉NO₆.HCl); rimexolone ((11β,16α,17β)-11-hydroxy-16,17-dimethyl-17-1-oxopropyl)androsta-1,4-dien-3-one); rofleponide ((22R)-6α,9α-Difluoro-11β,21-dihydroxy-16α,17α-propylmethylenedioxypregn-4-ene-3,20-dione); tipredane ((11β,17α)-17(ethylthio)-9α-fluoro-11β-hydroxy-17-(methylthio) androsta-1,4-dien-3-one); tixocortol ((11β)-11,17-dihydroxy-21-mercaptopregn-4-ene-3,20-dione), or its 21-pivalate form (C₂₆H₃₈O₅S; (11β)-21-[(2,2-dimethyl-1-oxopropyl)thio]-11,17-dihydroxypregn-4-ene-3,20-dione); triamcinolone ((11β,16α)-9-fluoro-11,16,17,21-tetrahydroxypregna-1,4-diene-3,20-dione), or its 16,21-diacetate form (C₂₅H₃₁FO₈; (11β,16α)-16,21-bis(acetyloxy)-9-fluoro-11,17-dihydroxypregna-1,4-diene-3,20-dione); Triamcinolone acetonide ((11β,16α)-9-fluoro-11,21-dihydroxy-16,17-[1-methylethylidenebis(oxy)]pregna-1,4-diene-3,20-dione), its 21-acetate crystal form, its 21-disodium phosphate form (C₂₄H₃₀FNa₂O₉P), or its 21-hemisuccinate form (C₂₈H₃₅FO₉); triamcinolone benetonide ((11β,16α)-21-[3-(benzoylamino)-2-methyl-1-oxopropoxy]-9-fluoro-11-hydroxy-16,17-[(1-methylethylidene)bis(oxy)]pregna-1,4-diene-3,20-dione); or triamcinolone hexacetonide; ((11β,16α)-21-(3,3-dimethyl-1-oxobutoxy)-9-fluoro-11-hydroxy-16,17-[(1-methylethylidene)bis(oxy)]pregna-1,4-diene-3,20-dione), analogues thereof, or pharmaceutically or veterinarily acceptable salts thereof.

35. The composition of claim 1, wherein the second agent comprises a glucocorticoid steroid selected from budesonide, testosterone, progesterone, estrogen, flunisolide, triamcinolone, beclomethasone, betamethasone, dexamethasone, fluticasone, methylprednisolone, prednisone, hydrocortisone, or mometasone.

36. The composition of claim 1, wherein the first active agent comprises a single stranded anti-sense DNA oligo.

37. The composition of claim 1, wherein the first active agent comprise(s) a double stranded DNA oligo.

38. The composition of claim 1, wherein the first active agent comprises a single stranded anti-sense RNA oligo(s).

39. The composition of claim 1, wherein the first active agent comprises a double stranded RNA oligo(s)

40. The composition of claim 1, which is a systemic or topical formulation.

41. The formulation of claim 40, selected from oral, intrabuccal, intrapulmonary, rectal, intrauterine, intratumor, intracranial, nasal, intramuscular, subcutaneous, intravascular, intrathecal, inhalable, transdermal, intradermal, intracavitary, implantable, iontophoretic, ocular, vaginal, intraarticular, otical, intravenous, intramuscular, intraglandular, intraorgan, intralymphatic, implantable, slow release or enteric coating formulations.

42. The formulation of claim 41, which is an oral formulation, wherein the carrier is selected from solid or liquid carriers.

43. The formulation of claim 42, in the form of a powder, dragees, tablets, capsules, sprays, aerosols, solutions, suspensions and emulsions, or optionally oil-in-water or water-in-oil emulsions.

44. The formulation of claim 41, which is a topical formulation, in the form of cream, gel, ointment, spray, aerosol, patch, solution, suspension or emulsion.

45. The formulation of claim 41, which is an injectable formulation, in the form of an aqueous or alcoholic solution or suspension, an oily solution or suspension, or an oil-in-water or water-in-oil emulsion.

46. The formulation of claim 41, in the form of a rectal or vaginal formulation, optionally a suppository.

47. The formulation of claim 41, in the form of a transdermal formulation, wherein the carrier comprises an aqueous or alcoholic solution, an oily solution or suspension, or an oil-in-water or water-in-oil emulsion.

48. The formulation of claim 47, in the form of an iontophoretic transdermal formulation, wherein the carrier comprises an aqueous or alcoholic solution, an oily solution or suspension, or an oil-in-water or water-in-oil emulsion, and wherein the formulation further comprises a transdermal transport promoting agent.

49. The formulation of claim 41, in the form of an implant, a capsule, a cartridge or a blister.

50. The formulation of claim 49, in the form of an aqueous or alcoholic solution or suspension, an oily solution or suspension, or an oil-in-water or water-in-oil emulsion.

51. The formulation of claim 40, wherein the carrier comprises a hydrophobic carrier.

52. The formulation of claim 51, wherein the carrier comprises lipid vesicles, optionally liposomes; or particles, optionally microcrystals.

53. The formulation of claim 52, wherein the carrier comprises liposomes, and the liposomes comprise the active agent(s).

54. The formulation of claim 41, which is a respirable or inhalable formulation, optionally aerosolizable or sprayable of particle size about 0.05 to about 10 micron.

55. The formulation of claim 54, having a particle size about 0.1 to about 5 micron.

56. The formulation of claim 41, which is a nasal or intrapulmonary formulation, optionally aerosolizable or sprayable of particle size about 8 to about 200 micron.

57. The formulation of claim 56, of particle size about 10 to about 50 micron.

58. The formulation of claim 41, in single or multiple unit form.

59. The formulation of claim 41, in bulk.

60. A therapeutic or prophylactic kit, comprising a delivery device; in separate containers, the active agent(s) of claim 1; and instructions for adding a carrier and preparing a formulation and for use of the kit.

61. The kit of claim 60, wherein the device delivers single metered doses of the formulation.

62. The kit of claim 60, wherein the formulation is a respirable formulation, and the delivery device comprises a nebulizer or a dry powder inhaler.

63. The kit of claim 62, wherein the device comprises a nebulizer or an insufflator and the formulation is provided in a piercable or openable capsule or cartridge.

64. The kit of claim 60, wherein the delivery device comprises a pressurized inhaler and the agent(s) is (are) provided as a suspension, solution or dry formulation of the active agent(s).

65. The kit of claim 60, further comprising, in a separate container, an agent selected from other therapeutic agents, surfactants, anti-oxidants, flavoring agents, fillers, volatile oils, dispersants, antioxidants, propellants, preservatives, buffering agents, RNA inactivating agents, cell-internalized or up-taken agents or coloring agents.

66. The kit of claim 60, comprising, in separate containers, one or more oligos, one or more AIS of formula (Ia), or (Ib) one or more surfactants, a carrier or diluent, optionally other therapeutic agents, and instructions for scheduling the administration of first and second agents.

67. The kit of claim 66, further comprising one or more ubiquinone(s), and instructions for scheduling the administration of first and second agents.

68. The kit of claim 60, wherein the device is a transdermal delivery device, and the kit further comprises a transdermal delivery agent, a transdermal carrier or diluent, and instructions for preparing and delivering a transdermal delivery formulation.

69. The kit of claim 60, wherein the device is an iontophoretic delivery device, and the kit further comprises an iontophoretic agent(s) and instructions for preparing and delivering an iontophoretic formulation.

70. The kit of claim 60, comprising, in separate containers, one or more oligo(s), one or more ubiquinone(s), one or more surfactants, a carrier or diluent, optionally other therapeutic agents, and instructions for scheduling the administration of first and second agents.

71. A method of preventing or treating a respiratory, lung or malignant disease or condition, comprising simultaneously, sequentially or separately administering to a subject in need of treatment, preventative, prophylactic or therapeutic amounts of the first and second active agents of claim 1.

72. The method of claim 71, wherein the oligo(s) and the AIS are administered in amounts effective for alleviating bronchoconstriction and/or lung inflammation or allergy(ies) and/or surfactant depletion or hyposecretion.

73. The method of claim 71, wherein the oligo(s) and the ubiquinone(s) are administered in amounts effective for alleviating bronchoconstriction, lung inflammation or allergies, or ubiquinone or lung surfactant depletion.

74. The method of claim 71, wherein one or more of the agent(s) is (are) administered as a nasal, inhalable, respirable or intrapulmonary composition(s) into the subject's respiratory system.

75. The method of claim 74, wherein one or more of the agents are administered intrapulmonarily or by inhalation.

76. The method of claim 74, wherein the respirable or inhalable composition(s) comprise(s) particles about 0.05 to about 10 micron in size.

77. The method of claim 74, wherein the nasal or intrapulmonary composition comprises particles about 8 to about 100 micron in diameter.

78. The method of claim 74, wherein the composition(s) is (are) administered as a respirable aerosol.

79. The method of claim 71, wherein the ubiquinone(s) is (are) administered orally, and the oligo(s) and the AIS are administered through the respiratory tract.

80. The method of claim 71, wherein the disease or condition is associated with pulmonary obstruction, bronchoconstriction, lung inflammation or allergy(ies), adenosine hypersensitivity, adenosine or adenosine receptor(s), hyperproduction, or surfactant or ubiquinone hypoproduction.

81. The method of claim 71, wherein the disease or condition comprises pulmonary vasoconstriction, respiratory inflammation or allergies, asthma, impeded respiration, respiratory distress syndrome (RDS), lung pain, cystic fibrosis (CF), allergic rhinitis (AR), apnea, pulmonary hypertension, emphysema, chronic obstructive pulmonary disease (COPD), pulmonary transplantation rejection, pulmonary fibrosis, pulmonary infections, bronchitis, or cancer.

82. The method of claim 71, wherein the disease or condition is associated with respiratory allergies, and the first active agent(s) is anti-sense to the initiation codon, the coding region, the 5′-end or the 3′-end genomic flanking regions, the 5′ or 3′ intron-exon junctions, or regions within 2 to 10 nucleotides of the junctions of at least one gene(s) encoding, or regulating expression of, an immunoglobulin(s), antibody(ies), or immunoglobulin or antibody receptors, or are anti-sense to the immunoglobulin(s), antibody(ies), or immunoglobulin or antibody receptor mRNA; MTAs of the oligo(s) or combinations thereof.

83. The method of claim 71, wherein the disease or condition is associated with a malignancy or cancer, and the oligo is anti-sense to the initiation codon, the coding region, the 5′-end or the 3′-end genomic flanking regions, the 5′ or 3′ intron-exon junctions, or regions within 2 to 10 nucleotides of the junctions of an oncogene(s) or at least one gene that regulates expression of, or encodes, a malignancy associated protein, or is(are) anti-sense to the oncogene or malignancy associated mRNA; MTAs or combinations thereof.

84. The method of claim 71, wherein the composition is administered transdermally or systemically.

85. The method of claim 71, wherein the composition is administered orally, intracavitarily, intranasally, intraurethral, intracavernous, intraanally, intravaginally, intrauterally, intraarticularly, transdermally, intrabucally, intravenously, subcutaneously, intramuscularly, intravascularly, intratumorously, intraglandularly, intraocularly, intracranial, into an organ, intravascularly, intrathecally, intralymphatically, intraotically, by implantation, by inhalation, intradermally, intrapulmonarily, intraotically, by slow release, by sustained release and by a pump.

86. The method of claim 71, wherein the mammal(s) is a human or non-human mammal.

87. The method of claim 71, wherein the oligo(s) is (are) administered in amount of about 0.005 to about 150 mg/kg body weight.

88. The method of claim 71, wherein the oligo(s) contain(s) up to about 15% A.

89. The method of claim 71, wherein the oligo(s) is (are) substantially free of A.

90. The method of claim 71, wherein the target comprises transcription factors, stimulating or activating factors, interleukins, interleukin receptors, chemokines, chemokine receptors, endogenously produced specific or non-specific enzymes, immunoglobulins, antibody receptors, central nervous system (CNS) or peripheral nervous or non-nervous system receptors, CNS and peripheral nervous and non-nervous system peptide transmitters, adhesion molecules, defensines, growth factors, microbial targets, vasoactive peptides, peptide receptors or binding proteins, or malignancy associated proteins.

91. The method of claim 71, wherein one or more As in the oligo(s) is(are) substituted by a universal base that comprise(s) a heteroaromatic base(s) that bind(s) to thymidine or uridine but has(have) less than about 0.3 of the adenosinebase agonist or antagonist activity at an adenosine A₁, A₂ₐ, A_{2b} or A₃ receptor.

92. The method of claim 91, wherein the heteroaromatic base(s) comprise(s) pyimidines or purines, which may be substituted by O, halo, NH₂, SH, SO, SO₂, SO₃, COOH, branched or fused primary or secondary amino, alkyl alkenyl alkynyl, cycloalkyl, heterocycloalkyl, aryl, heteroaryl, alkoxy, alkenoxy, acyl cycloacyl, arylacyl alkynoxy, cycloalkoxy, aroyl, arylthio, arylsulfoxyl, halocycloalkyl, alkylcycloalkyl, alkenylcycloalkyl, alkynylcycloalkyl, haloaryl, alkylaryl, alkenylaryl, alkynylaryl, arylalkyl, arylalkenyl, arylalkyl, arylcycloalkyl, all of which may be further substituted by O, halo, NH₂, primary, secondary or tertiary amine, SH, SO, SO₂, SO₃, cycloalkyl, heterocycloalkyl or heteroaryl.

93. The method of claim 91, wherein the purines are substituted at positions 1, 2, 3, 6, and/or 8, the pyrimidines are substituted at positions 2, 3, 4, 5 and/or 6 and have the chemical formula 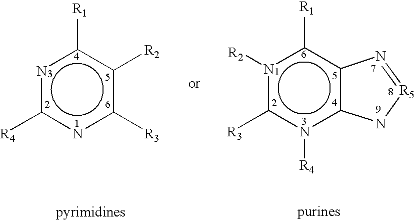
wherein R₁, R², R³, R⁴ and R⁵ are independently H, alkyl, alkenyl or alkynyl and R³ is H, aryl dicycloalkyl dicycloalkenyl, dicycloalkynyl cycloakyl, cycloalkenyl, cycloalkynyl, O-cycloalkyl, O-cycloalkenyl, O-cycloalkynyl, NH₂-alkylamino-ketoxyalkyloxy-aryl, or mono or dialkylaminoalkyl-N-alkylamino-SO₂aryl, and R⁴ and R⁵ are independently R¹ and together are R³, and the pyrimidines and purines optionally comprise theophylline, caffeine, dyphylline, etophylline, acephylline piperazine, bamifylline, enprofylline or xanthine.

94. The method of claim 93, wherein the universal base(s) comprise(s) 3-nitropyrrole-2′-deoxynucleoside, 5-nitro-indole, 2-deoxyribosyl-(5-nitroindole), 2-deoxyribofuranosyl-(5-nitroindole), 2′-deoxyinosine, 2′-deoxynebularine, 6H, 8H-3,4-dihydropyrimido[4,5-c]oxazine-7-one, or 2-amino-6-methoxyaminopurine.

95. The method of claim 71, wherein the second active agent comprises an AIS of formula (Ia) selected from dehydroepiandrosterone, 16-alphabromodehydroepiandrosterone, 16-alpha-fluorodehydroepiandrosterone, etiocholanolone, dehydroepiandrosterone sulfate or other pharmaceutically or veterinarily acceptable salts thereof.

96. The method of claim 71, wherein the second active agent comprises an AIS formula (Ib), wherein R¹⁵ and R¹⁶ together are ═O; R⁵ is —OH; R⁵ is —SO₂R²⁰; R¹⁵ and R²⁰ together is H; or pharmaceutically or veterinarily acceptable salts thereof.

97. The method of claim 71, wherein the active agents are present in an amount of about 0.01 to about 99.99 w/w of the composition.

98. The method of claim 71, wherein the second active agent comprises an AIS selected from 21-acetoxypregnenolone ((3β)-21-(acetyloxy)-3-hydroxypregn-5-en-20-one); alclometasone ((7α,11β,16α)-7-Chloro-11,17,21-trihydroxy-16-methylpregna-1,4-diene-3,20-dione), or its 17,21-dipropionate form (C₂₈H₃₇ClO₇); algestone ((16α)-16,17 dihydroxypregn-4-ene-3,20-dione), its cyclic acetal with acetone form (C₂₈H₃₄O₄), or its 16α-methyl ether form (C₂₂H₃₂O₄); amcinonide ((11β, 16α)-21-(acetyloxy)-16,17-[cyclopentylidenebis(oxy)]-9-fluoro-11-hydroxypregna-1,4-di-ene-3,20-dione); beclomethasone ((11β,16β)-9-chloro-11,17,21-trihydroxy-16-methylpregna-1,4-diene-3,20-dione), its dipropionate form (C₂₈H₃₇ClO₇), or its monopropionate form; betamethasone ((11β,16β)-9-fluoro-11,17,21-trihydroxy-16-methylpregna-1,4-diene-3,20-dione), its 21-acetate form (C₂₄H₃₁FO₆), its 21-adamantoate form (C₃₃H₄₃FO₆), its 17-benzoate form (C₂₉H₃₃FO₆), its 17,21-dipropionate form (C₂₈H₃₇FO₇), its 17-valerate form (C₂₇H₃₇FO₆), or its 21-phospate disodium salt form (C₂₂H₂₈FNa₂O₈P); budesonide ((11β,16α)-16,17-[butylidenebis(oxy)]-11,21-dihydropregna-1,4-diene-3,20-dione); chloroprednisone ((6α)-chloro-17,21-dihydroxypregna-1,4-diene-3,11,20-trione), or its 21-acetate from (C₂₃H₂₇CO₆); ciclesonide; clobetasol ((11β,16β)-21-chloro-9-fluoro-11,17-dihydroxy-16-methylpregna-1,4-diene-3,20-dione), or its 17-propionate form (C₂₅H₃₂ClFO₅); clobetasone ((16β)-21-chloro-9-fluoro-17-hydroxy-16-methylpregna-1,4-diene-3,11,20-trione), or its 17-butyrate form (C₂₆H₃₂ClFO₅); clocortolone ((6α,11β,16α)-9-chloro-6-fluoro-11,21-dihydroxy-16-methylpregna-1,4-diene-3,20-dione), its 21-acetate form (C₂₄H₃₀ClFO₅), or its 21-pivalate form (C₂₇H₃₆ClFO5); cloprednol ((11β)-6-chloro-11,17,21-trihydroxypregna-1,4,6-triene-3,20-dione); coroxon (phosphoric acid 3-chloro-4-methyl-2-oxo-2H-1-benzopyran-7-yl diethyl ester); cortisone (17,21-dihydroxypregn-4-ene-3,11,20-trione), its 21-acetate form (C₂₃H₃₀O₆), or its 21-cyclopentanepropionate form (C₂₉H₄₀O₆); cortivazol ((11β,16α)-21-(acetyloxy)-11,17-dihydroxy-6,16-dimethyl-2′-phenyl-2′H-pregna-2,4,6-trieno[3,2-c]pyrazol-20-one); deflazacort ((11β,16β)-21-(acetyloxy)-11-hydroxy-2′-methyl-5′H-pregna-1,4-dieno[17,16-d]oxazole-3,20-dione); desonide ((11β,16α)11,21-dihydroxy-16,17-[(1-methylethylidene)bis(oxy)]pregna-1,4-diene-3,20-dione); desoximetasone ((11β,16α)-9-fluoro-11,21-dihydroxy-16-methylpregna-1,4-diene-3,20-dione); dexamethasone ((11β,16α)-9-fluoro-11,17,21-trihydroxy-16-methylpregna-1,4-diene-3,20-dione), its 21-acetate form (C₂₄H₃₁FO₆), its 21-(3,3-dimethylbutyrate) form (C₂₈H₃₉FO₆; Chemerda et al., U.S. Pat. No. 2,939,873), its 21-diethylaminoacetate form (C₂₈H₄₁FNO₆), its 21-isonicotinate form (C₂₈H₄₁FNO₆), its 17,21-dipropionate form (C₂₈H₃₇FNO₆), or its 21-palmitate form (C₃₈H₅₉FO₆); diflorasone ((6α,11β,16β)-6,9-difluoro-11,17,21-trihydroxy-16-methylpregna-1,4-diene-3,20-dione), or its diacetate form (C₂₆H₃₂F₂O₇); diflucortolone ((6α,11β,16α)-6,9-difluoro-11,21-dihydroxy-16-methylpregna-1,4-diene-3,20-dione), or its 21-valerate form (C₂₇H₃₆F₂O₅); difluprednate ((6α,11β)-21-(acetyloxy)-6,9-difluoro-11-hydroxy-17-(1-oxobutoxy)pregna-1,4-diene-3,20-dione); enoxolone ((3β,20β)-3-hydroxy-11-oxoolean-12-en-29-oic acid), or its 18α-hydrogen form; fluazacort ((11β,16β)-21-(acetyloxy)-9-fluoro-11-hydroxy-2′-methyl-5′H-pregna-1,4-dieno[17,16-d]oxazole-3,20-dione); flucloronide ((6α,11β,16α)-9,11-dichlro-6-fluoro-21-hydroxy-16,17-[(1-methylethylidene)bis(oxy)]-pregna-1,4-diene-3,20-dione); flumethasone ((6α,11β,16α)-6,9 difluoro-11,17,21-trihydroxy-16-methylpregna-1,4-diene-3,20-dione), its 21-acetate form (C₂₄H₃₀F₂O₆), or its 21-pivalate form (C₂₇H₃₆F₂O₆); flunisolide ((6α,11β,16α)-6-fluoro-11,21-dihydroxy-16,17-[(1-methylethylidene) bis(oxy)]pregna-1,4-diene-3,20-dione), or its 21-acetate form (C₂₆H₃₃FO₇); fluocinolone acetate((6α,11β,16α)-6,9-difluoro-11,21-dihydroxy-16,17-[(1-methylethylidene)bis(oxy)]-pregna-1,4-diene-3,20-dione); fluocinonide ((6α,11β,16α)-21-acetyloxy)-6,9-difluoro-11-hydroxy-16,17-[(1-methylethylidene)bis(oxy)]-pregna-1,4-diene-3,20-dione); fluocortin butyl((6α,11β,16α)-6-fluoro-11-hydroxy-16-methyl-3,20-dioxopregna-1,4-dien-21-oic acid butyl ester); fluocortolone ((6α,11β,16α)-6-fluoro-11,21-dihydroxy-16-methylpregna-1,4-diene-3,20-dione), its 21-acetate form (C₂₄H₃₁FO₅), its 21-hexanoate form (C₂₈H₃₉FO₅), or its 21-pivalate form (C₂₂H₃₇FO₅); fluorometholone ((6α,11β)-9-fluoro-11,17-dihydroxy-6-methylpregana-1,4-diene-3,20-dione), or its 17-acetate form (C₂₄H₃₁FO₅); fluperolone acetate([11β,17α,17(S)]-17-[2-(acetyloxy)-1-oxopropyl]-9-fluoro-11,17-dihydroxyandrosta-1,4-dien-3-one); fluprednidene acetate((11β)-21-(acetyloxy)-9-fluoro-11,17-dihydroxy-16-methylenepregna-1,4-diene-3,20-dione); fluprednisolone ((6α,11β)-6-fluoro-11,17,21-trihydroxypregna-1,4-diene-3,20-dione), or its 21-acetate form (C₂₃H₂₉FO₆); flurandrenolide ((6α,11β,16α)-6-fluoro-11,21-dihydroxy-16,17-[(1-methylethylidene)bis(oxy)]pregn-4-ene-3,20-dione); fluticasone propionate ((6α, 11β, 16α, 17α)-6,9-difluoro-11-hydroxy-16-methyl-3-oxo-17-(1-oxopropoxy)androsta-1,4-diene-17-carbothioic acid S-(fluoromethyl) ester); formocortal ((11β,16α)-21-(acetyloxy)-3-(2-chloroethoxy)-9-fluoro-11-hydroxy-16,17-[(1-methylethylidene)bis(oxy)]-20-oxopregna-3,5-diene-6-carboxaldehyde); halcinonide ((11β,16α)-21-chloro-9-fluoro-11-hydroxy-16,17-[(1-methyethylidene)bis(oxy)]pregn-4-ene-3,20-dione); halobetasol propionate (6α,11β,16β)-21-chloro-6,9-difluoro-11-hydroxy-16-methyl-17-(1-oxopropoxy)pregna-1,4-diene-3,20-dione); halometasone ((6α,11β,16α)-2-chloro-6,9 difluoro-11,17,21-trihydroxy-16-methylpregna-1,4-diene-3,20-dione), or its monohydrate form (C₂₂H₂₇ClF₂O₅.H₂O); halopredone acetate((6β,11β)-17,21-bis(acetyloxy)-2-bromo-6,9-difluoro-11-hydroxypregna-1,4-diene-3,20-dione); hydrocortamate (N,N-diethylglycine (11β)-11,17-dihydroxy-3,20-dioxopregn-4-en-21-yl ester), or its hydrochloride form (C₂₇H₄₁NO₆.HCl); hydrocortisone ((11β)-11,17,21-trihydroxypregn-4-ene-3,20-dione), its 21-acetate form (C₂₃H₃₂O₆), its 17-butyrate form (C₂₅H₃₆O₆), its 21-phosphate disodium salt form (C₂₁H₂₉Na₂O₈P), its 21-sodium succinate form (C₂₅H₃₃NaO₈), its 17-valerate form (C₂₆H₃₈O₆), or its cypionate form; loteprednol etabonate ((11β,17α)-17-[(ethoxycarbonyl)oxy]-11-hydroxy-3-oxoandrosta-1,4-diene-17-carboxylic acid chloromethyl ester); mazipredone ((11β)-11,17-dihydroxy-21-(4-methyl-1-piperazinyl)pregna-1,4-diene-3,20-dione), or its hydrochloride form (C₂₆H₃₈N₂O₄.HCl); medrysone ((6α,11β)-11-hydroxy-6-methylpregn-4-ene-3,20-dione); meprednisone ((16β)-17,21-dihydroxy-16-methylpregna-1,4-diene-3,11,20-trione), or its 21-acetate form (C₂₄H₃₀O₆); methylprednisolone ((6α,11β)-11,17,21-trihydroxy-6-methylpregna-1,4-diene-3,20-dione; Sebek and Spero, U.S. Pat. No. 2,897,218, and Gould, U.S. Patent No. 3,053,832), its 21-acetate form (C₂₄H₃₂O₆), its 21-phosphate disodium salt form (C₂₂H₂₉Na₂O₈P), its 21-succinate sodium salt form (C₂₆H₃₃NaO₈), or its aceponate form (C₂₇H₃₆O₇); mometasone furoate ((11β,16α)-9,21-dichloro-17-[(2-furanylcarbonyl)oxy]-11-hydroxy-16-methylpregna-1,4-diene-3,20-(ione); paramethasone ((6α,11β,16α)-6-fluoro-11,17,21-trihydroxy-16-methylpregna-1,4-diene-3,20-dione), its 21-acetate form (C₂₄H₃₄FO₆), its disodium phosphate form, or a mixture of its 21-acetate and disodium phosphate form; prednicarbate ((11β)-17[(ethoxycarbonyl)oxy]-11-hydroxy-21-(1-oxopropoxy)pregna-1,4-diene-3,20-dione); prednisolone ((11β)-11,17,21-trihydroxypregna-1,4-diene-3,20-dione), its 21-acetate form (C₂₃H₃₀O₆), its 21-tert-butylacetate form (C₂₇H₃₈O₆; Sarrett), its 21-hydrogen succinate form (C₂₅H₃₂O₈), its 21-succinate sodium salt form (C₂₅H₃₁NaO₈), its 21-stearoylgylcolate form (C₄₁H₆₄O₈), its 21-m-sulfobenzoate sodium salt form (C₂₈H₃₁,NaO₉S; (11β)-11,17-dihydroxy-21-[(3-sulfobenzoyl)oxy]pregna-1,4-diene-3,20-dione monosodium salt), or its 21-trimethylacetate form (C₂₆H₃₆O₆); prednisolone 21-diethylaminoacetate(N,N-diethylglycine (11β)-11,17-dihydroxy-3,20-dioxopregna-1,4-dien-21-yl ester; British Patent No. 862,370), or its hydrochloride form (C₂₇H₃₉NO₆.HCl); prednisolone sodium phosphate (11,17-dihydroxy-21-(phosphonooxy)pregna-1,4-diene-3,20-dione disodium salt); prednisone (17,21-dihydroxypregna-1,4-diene-3,11,20-trione), or its 21-acetate form (C₂₃H₂₈O₆); prednival ((11β)-11,21-dihydroxy-17-[(1-oxopentyl)oxy]pregna-1,4-diene-3,20-dione;), or its 21-acetate form (C₂₈H₃₈O₇); prednylidene ((11β)-11,17,21-trihydroxy-16-methylenepregna-1,4-diene-3,20-dione), or its 21-diethylaminoacetate hydrochloride form (C₂₈H₃₉NO₆.HCl); rimexolone ((11β,16α,17β)-11-hydroxy-16,17-dimethyl-17-(1-oxopropyl)androsta-1,4-dien-3-one); rofleponide ((22R)-6α,9α-Difluoro-11β,21-dihydroxy-16α,17α-propylmethylenedioxypregn-4-ene-3,20-dione); tipredane ((11β, 17α)-17-(ethylthio)-9α-fluoro-11β-hydroxy-17-(methylthio) androsta-1,4-dien-3-one); tixocortol ((11β)-11,17-dihydroxy-21-mercaptopregn-4-ene-3,20-dione), or its 21-pivalate form (C₂₆H₃₈O₅S; (11β)-21-[(2,2-dimethyl-1-oxopropyl)thio]-11,17-dihydroxypregn-4-ene-3,20-dione); triamcinolone ((11β,16α)-9-fluoro-11,16,17,21-tetrahydroxypregna-1,4-diene-3,20-dione), or its 16,21-diacetate form (C₂₅H₃₁FO₈; (11β,16α)-16,21-bis(acetyloxy)-9-fluoro-11,17-dihydroxypregna-1,4-diene-3,20-dione); Triamcinolone acetonide ((11β,16α)-9-fluoro-11,21-dihydroxy-16,17-[1-methylethylidenebis(oxy)]pregna-1,4-diene-3,20-dione), its 21-acetate crystal form, its 21-disodium phosphate form (C₂₄H₃₀FNa₂O₉P), or its 21-hemisuccinate form (C₂₈H₃₅FO₉); triamcinolone benetonide ((11β, 16α)-21-[3-(benzoylamino)-2-methyl-1-oxopropoxy]-9-fluoro-11-hydroxy-16,17-[(1-methylethylidene)bis(oxy)]pregna-1,4-diene-3,20-dione); or triamcinolone hexacetonide; ((11β,16α)-21-(3,3-dimethyl-1-oxobutoxy)-9-fluoro-11-hydroxy-16,17-[(1-methylethylidene) bis(oxy)]pregna-1,4-diene-3,20-dione), or pharmaceutically or veterinarily acceptable salts thereof.

99. The method of claim 71, wherein the second active agent comprises an AIS selected from budesonide, testosterone, progesterone, estrogen, flunisolide, triamcinolone, beclomethasone, betamethasone, dexamethasone, fluticasone, methylprednisolone, prednisone, hydrocortisone, or mometasone.

100. A method of enhancing the prophyllactic or therapeutic respiratory effect of an anti-inflammatory steroid in a subject, comprising administering to the subject, in addition to the AIS, the oligonucleotide(s) (oligo(s)) of claim 1, the AIS and the oligo(s) being administered in amounts effective for reducing or depleting levels of, or reducing sensitivity to, adenosine, reducing levels of adenosine receptors, producing bronchodilation, increasing levels of ubiquinone or lung surfactant in a subject's tissue (s), or treating bronchoconstriction, lung inflammation or lung allergies or a respiratory or lung disease or condition.

101. The method of claim 100, further administering to the subject a ubiquinone of the chemical formula.

102. The method of claim 100, wherein the steroid comprises budesonide, testosterone, progesterone, estrogen, flunisolide, triamcinolone, beclomethasone, betamethasone, dexamethasone, fluticasone, methylprednisolone, prednisone, hydrocortisone, or mometasone

103. The method of claim 100, wherein the oligo(s) is anti-sense to the initiation codon, the coding region, the 5′-end or the 3′-end genomic flanking regions, the 5′ or 3′ intron-exon junctions, and regions within 2 to 10 nucleotides of the junctions of at least one oncogene(s) and a gene(s) enclding or regulating expression of a target polypeptide(s) associated with lung airway dysfunction, or anti-sense to the corresponding mRNA and the polypeptide mRNA; combinations, MTAs or mixtures of the oligos; the polypeptides comprising peptide factors and transmitters, antibodies, cytokines or chemokines, enzymes, binding proteins, adhesion molecules, their receptors, or malignancy associated proteins.

104. The method of claim 100, further comprising administering to the subject other therapeutic or bioactive agents selected from analgesics, pre-menstrual medications, menopausal agents, anti-aging agents, anti-anxyolytic agents, mood disorder agents, anti-depressants, anti-bipolar mood agents, anti-schyzophrenic agents, anti-cancer agents, alkaloids, blood pressure controlling agents, muscle relaxants, steroids, soporific agents, anti-ischemic agents, anti-arrythmic agents, contraceptives, vitamins, minerals, tranquilizers, neurotransmitter regulating agents, wound healing agents, anti-angyogenic agents, cytokines, growth factors, B-adrenergic receptor agonists, anti-metastatic agents, antacids, anti-histaminic agents, anti-bacterial agents, anti-viral agents, anti-gas agents, appetite suppressants, sun screens, emollients, skin temperature lowering products, radioactive phosphorescent or fluorescent contrast diagnostic or imaging agents, libido altering agents, bile acids, laxatives, anti-diarrheic agents, skin renewal agents, hair growth agents, analgesics, premenstrual medications, anti-menopausal agents, hormones, anti-aging agents, anti-anxiolytic agents, nociceptic agents, mood disorder agents, anti-depressants, anti-bipolar mood agents, anti-schizophrenic agents, anti-cancer agents, alkaloids, blood pressure controlling agents, other hormones, other anti-inflammatory agents, agents for treating arthritis, burns, wounds, chronic bronchitis, chronic obstructive pulmonary disease (COPD), inflammatory bowel disease such as Crohn's disease, ulcerative colitis, autoimmune disease, or lupus erythematosus, muscle relaxants, soporific agents, anti-ischemic agents, anti-arrhythmic agents, contraceptives, vitamins, minerals, tranquilizers, neurotransmitter regulating agents, wound and burn healing agents, anti-angiogenic agents, cytokines, growth factors, anti-metastatic agents, antacids, anti-histaminic agents, anti-bacterial agents, anti-viral agents, anti-gas agents, agents for reperfusion injury, counteracting appetite suppressants, sun screens, emollients, skin temperature lowering products, radioactive phosphorescent or fluorescent contrast diagnostic or imaging agents, libido altering agents, bile acids, laxatives, anti-diarrheic agents or skin renewal agents.

105. The method of claim 100, wherein the oligo(s) and/or the steroid(s) is(are) administered with surfactant protein A, surfactant protein B, surfactant protein C, surfactant protein D and surfactant Protein E, di-saturated phosphatidyl choline (other than dipalmitoyl), dipalmitoyl phosphatidyl choline, phosphatidyl choline, phosphatidyl glycerol phosphatidyl inositol, phosphatidyl ethanolamine, phosphatidyl serine; phosphatidic acid, ubiquinones, lysophosphatidyl ethanolamine, lysophosphatidyl choline, palmitoyl-lysophosphatidyl choline, dehydroepiandrosterone, dolichols, sulfatidic acid, glycerol-3-phosphate, dihydroxyacetone phosphate, glycerol, glycero-3-phosphocholine, dihydroxy acetone, palmitate, cytidine diphosphate (CDP) diacyl glycerol, CDP choline, choline, choline phosphate; natural or artificial lamellar bodies as carrier surfactant vehicles, omega-3 fatty acids, polyenic acid, polyenoic acid, lecithin, palmitinic acid, non-ionic block copolymers of ethylene or propylene oxides, polyoxypropylene, monomeric or polymeric, polyoxyethylene, monomeric and polymeric, poly (vinyl amine) with dextran and/or alkanoyl side chains, Brij 35, Triton X-100 or synthetic surfactants ALEC, Exosurf, Survan or Atovaquone.

106. The method of claim 100, wherein the AIS comprises a steroid of chemical formula (Ia) or (Ib).

107. The method of claim 106, wherein the AIS is selected from budesonide, testosterone, progesterone, fluticasone, beclomethasone, prednisone, momethasone, estrogen, dexamethasone, hydrocortisone, triamcinolone, flunisolide, methylprednisolone prednisone, hydrocortisone, or analogues thereof.

108. The method of claim 100, wherein the first and second active agents are administered systemically or topically.

109. The method of claim 100, wherein the first and second active agents are administered as an oral intrabuccal, intrapulmonary, rectal, intrauterine, intratumor, intracranial, nasal, intramuscular, subcutaneous, intravascular, intrathecal, inhalable, transdermal, intradermal, intracavitary, implantable, iontophoretic, ocular, vaginal, intraarticular, otical intravenous, intramuscular, intraglandular, intraorgan, intralymphatic, implantable, slow release or enteric coating formulation.

110. The method of claim 101, wherein the ubiquinone is administeredorally.

107. The method of claim 106, wherein the oligo(s) and the AIS is(are) administered intrapulmonarily, into the respiration, nasally, or by inhalation.

108. The method of claim 106, wherein the oligo(s) or the AIS is(are) administered as a respirable or inhalable formulation, optionally an aerosol of particle size about 0.05 to about 10 micron.

109. The method of claim 107, wherein the formulation comprises an oligo(s) or AIS of particle size about 0.1 micron to about 5 micron.

110. The method of claim 106, wherein the oligo(s) or the AIS is(are) administered nasallym intrapulmonarily, optionally an aerosol of particle size about 8 to about 100 micron.

111. The method of claim 109, wherein the oligo(s) or the AIS has(have) a particle size about 10 to about 50 micron.
